SEQUENCE LISTING (12 sequences)

SEQ ID NO: 1 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 2 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 4 - protein, 133 aa, Homo sapiens [sequence omitted]

SEQ ID NO: 5 - protein, 149 aa, Mus sp.
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 6 - protein, 133 aa, synthetic construct
APTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 132 aa, synthetic construct
PTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 133 aa, synthetic construct
APTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 9 - protein, 132 aa, synthetic construct
PTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 10 - protein, 6 aa, synthetic construct
HHHHHH

SEQ ID NO: 11 - protein, 8 aa, synthetic construct
HHHHHHHH

SEQ ID NO: 12 - protein, 6 aa, synthetic construct
  VARIANT 1..6: note = This sequence may encompass 3-6 repeating units
HHHHHH

(19) United States
(12) Patent Application Publication
(10) Pub. No.: US 2025/0288666 A1
(43) Pub. Date: Sep. 18, 2025

(54) METHODS AND COMPOSITIONS FOR USE IN CELL THERAPY OF NEOPLASTIC DISEASE

(71) Applicant: Synthekine, Inc., Menlo Park, CA (US)

(72) Inventors: Christina Kochel, Menlo Park, CA (US); Richard B. Murphy, Menlo Park, CA (US); Martin Oft, Menlo Park, CA (US); Paul-Joseph Penaflor-Aspuria, Menlo Park, CA (US)

(21) Appl. No.: 18/576,329

(22) PCT Filed: Jul. 14, 2022
(86) PCT No.: PCT/US2022/073746

§ 371 Date: Jan. 3, 2024

Related U.S. Application Data
(60) Provisional application No. 63/221,857, filed on Jul. 14, 2021.

(51) Int. Cl. A61K 40/11; A61K 35/17; A61K 38/20; A61K 40/42; A61K 45/06; A61P 35/00; C12N 5/0783; C12N 15/867

(52) U.S. Cl. CPC A61K 40/11; A61K 35/17; A61K 38/2013; A61K 40/428; A61K 45/06; A61P 35/00; C12N 5/0636; C12N 15/867; C12N 2501/2302

(57) ABSTRACT

The present disclosures relates to methods of use to the use of interleukin-2 (IL2) muteins, pharmaceutical formulations thereof, methods useful in the treatment of human disease in combination with adoptive cell therapy. In particular, the present disclosure provides compositions and methods for the use of αβhIL2 muteins that selectively stimulate the proliferation of antigen experienced T cells ex vivo and optionally in vivo; methods of use of αβhIL2 muteins for the activation and expansion of antigen experienced T cells in an isolated population of cells; methods of use of αβhIL2 muteins ex vivo for the to prepare a population of cells enriched for antigen experienced T cells and administering the population of cells to a subject; methods of use of αβhIL2 mutein ex vivo to prepare a population of cells enriched for antigen experienced T cells and administering the population of cells to a subject and administering to said subject a therapeutically effective amount of a αβhIL2 mutein (e.g., such that the administered population of cells proliferate and have a therapeutic effect) and compositions comprising populations T cells of enriched for antigen activated cells.

### CROSS REFERENCES TO RELATED APPLICATIONS

This application is a U.S. National Stage of PCT/US2022/073746, International Filing Date Jul. 14, 2022 which claims the priority of U.S. Provisional Application Ser. No. 63/221,857 filed Jul. 14, 2021, the disclosure of which is herein incorporated by reference in their entirety for all purposes.

### REFERENCE TO AN ELECTRONIC SEQUENCE LISTING

The contents of the electronic sequence listing (106249-1334019-002710PC_SL.xml; Size: 13,678 bytes; and Date of Creation: Jan. 2, 2024) is herein incorporated by reference in its entirety.

### BACKGROUND OF THE DISCLOSURE

Adoptive cell therapy, in particular therapy with tumor infiltrating lymphocytes (TILs) or “TIL therapy” is a therapeutic modality having significant documented efficacy in the treatment of neoplastic disease in human subjects. See, e.g., Rosenberg (U.S. Pat. No. 5,126,132A issued Jun. 30, 1992 and Spiess, et al. (1987) J Natl Cancer Inst 79:1067-1075. In typical current practice, human TIL therapy consists of: (1) isolation of a population of cells from a subject, the population of cells comprising tumor infiltrating lymphocytes (TILs), (2) ex vivo expansion and activation of the isolated cell population, and (3) and reinfusion of the expanded activated cell population. Frequently, the patient is treated with a preparative lymphodepleting regimen prior to reinfusion of the cells and administration of human interleukin-2 (hIL2) in combination with the reinfusion of the cell population. The preparative lymphodepleting regimen depletes a variety of immune cells including Tregs and removes cellular “sinks” and is associated with improved antitumor efficacy. The systemic administration of IL2 supports the persistence of the re-infused TILs in vivo. In typical clinical practice, shortly after infusion of the TILs, the patient receives intravenous hIL2 at a dose of 720,000 IU/kg every 8 hours until maximal tolerance commonly referred to as high-dose IL2 therapy. This administration of hIL2 subsequent to the reinfusion of the expanded cell population and is thought to further enhance the survival and clinical efficacy of the TILs.

Subjects suffering from metastatic melanoma treated in substantial accordance with this regimen obtained objective tumor responses of approximately 50% in several phase I/II clinical trials. Rosenberg, et al. (2011) Clin Cancer Res 17:4550-4557; Andersen, et al. (2016) Clin Cancer Res 22:3734-3745; and Besser, et al. (2013) Clin Cancer Res 19:4792-4800. Building on the success of TIL therapy observed in melanoma patients, others demonstrated that it is possible to obtain TILs from a wide variety of other tumor types including, but not limited to, cervical cancer (Stevanovic, et al. (2015) J Clin Oncol 33:1543-1550), renal cell cancer (Andersen, et al. (2018) Cancer Immunol Res 6:222-235), breast cancer (Lee, et al. (2017) Oncotarget 8:113345-113359), non-small cell lung cancer (Ben-Avi, et al. (2018) Cancer Immunol Immunotherapy 67:1221-1230) gastrointestinal cancers (Turcotte (2013) J Immunol 191:2217-2225 and Turcotte et al (2014) Clin Cancer Res 20:331-343), cholangiocarcinoma (Tran, et al. (2014) Science 344:641-645), pancreatic cancer (Hall, et al. (2016) J Immunother Cancer 4:61) head and neck cancer (Junker, et al. (2011) Cytotherapy 13:822-834) and ovarian cancer (Fujita, et al. (1995) Clin Cancer Res 1: 501-507).

A significant advantage of TIL therapy is that it results in a broad polyclonal response to both defined and novel tumor antigens and in the context of all possible MHC molecules as opposed to the monoclonal specificity of TCR or CAR T-cells. Additionally, the “on target/off-tumor” toxicity which is a problem associated with genetically modified T-cell therapies (such as CAR-T cells) is less frequently observed in TIL therapy. TILs recognize the neoantigens that arise as a consequence of tumor-specific mutations and studies suggest that such neoantigen-reactive T cells are likely the dominant player inducing tumor regressions after TIL therapy. Consequently, it is expected that TIL therapy will be particularly efficacious in tumors with high mutation rates such as skin and small cell lung cancers, tumors with microsatellite instability or mismatch repair-deficiency, and tumors of viral origin.

Two current methods of ex vivo expansion and activation of TILs are used: the “selected TIL” method and the “young” TIL method.

The “selected TIL” method is a more traditional approach and involves ex vivo expansion of TILs in two stages: a first stage in which TILs from tumor fragments are maintained in the presence of high dose IL2 for a period of 4-5 weeks and second stage in which the particular subsets of TILs that demonstrate IFNy secretion in response to the exposure of autologous tumor cells are expanded and a second stage involving a “rapid expansion protocol” or “REP” using soluble anti-CD3 mAbs in the presence of an excess (e.g. 200:1 ratio) of irradiated PBMC feeder cells (either autologous or allogeneic feeders) for two days followed by culture in the presence of IL2 for an additional 12 days. A typical REP results in 1,000-fold to 2,000-fold expansion of TILs during the 2-week culture period. Using current methods, approximately, 5×10⁷ pre-REP TILs are needed to obtain the required number of cells for a typical course of TIL therapy.

More recent TIL preparation protocols known as the “young TIL” methods reduce the initial expansion before the cells are subjected to the REP avoid the selection step based on tumor reactivity and rather use bulk unselected TILs for REP expansion. Reports suggest that the overall response using such “young TIL” methods is similar to that reported by the “selected” TIL approach in refractory melanoma patients however such young TIL products likely have a lower percentage of tumor reactive T cells as may correlate with lower anti-tumor and there has been no direct controlled comparison of the young TIL with the selected TIL method in clinical trial with large number of patients.

hIL2 is a pluripotent cytokine that a wide spectrum of effects on the immune system and plays important roles in regulating both immune activation, suppression and homeostasis. The property of hIL2 to promote the proliferation and expansion of activated T lymphocytes makes it particularly is useful in TIL therapy protocols and is used in both the ex vivo and in vivo phases of the current practice of TIL therapy in human subjects. The consensus amino acid sequence of wild-type human IL2 is found in Genbank under accession locator NP_000577.2.

Human IL2 exerts its intracellular signaling activities on T cells via its interaction with two IL2 receptor signaling complexes: (a) an “intermediate affinity” IL2 receptor comprising CD122 and CD132 (also referred to as “IL2Rβγ”) and (b) a “high affinity” IL2 receptor complex comprising the CD25, CD122 and CD132 proteins (also referred to as “IL2Rαβγ”).

CD25 is a 55 kD polypeptide that is constitutively expressed in Treg cells and inducibly expressed on other T cells in response to activation (e.g., by CD3). CD25 is also referred to in the literature as the “low affinity” IL2 receptor. hIL2 binds to hCD25 with a Kd of approximately 10⁻⁸M. The human CD25 is expressed as a 272 amino acid pre-protein comprising a 21 amino acid signal sequence which is post-translationally removed to render a 251 amino acid mature protein. Amino acids 22-240 (amino acids 1-219 of the mature protein) correspond to the extracellular domain. Amino acids 241-259 (amino acids 220-238 of the mature protein) correspond to transmembrane domain. Amino acids 260-272 (amino acids 239-251 of the mature protein) correspond to intracellular domain. The intracellular domain of CD25 is comparatively small (13 amino acids) and has not been associated with any independent signaling activity. The IL2/CD25 complex has not been observed to produce a detectable intracellular signaling response. The consensus human CD25 nucleic acid and protein sequences may be found as Genbank accession numbers NM__000417 and NP_0004Q8, respectively.

CD122 is a single pass type I transmembrane protein. The human CD122 (hCD122) is expressed as a 551 amino acid protein, the first 26 amino acids comprising a signal sequence which is post-translationally cleaved in the mature 525 amino acid protein. Amino acids 27-240 (amino acids 1-214 of the mature protein) correspond to the extracellular domain, amino acids 241-265 (amino acids 225-239 of the mature protein) correspond to the transmembrane domain and amino acids 266-551 (amino acids 240-525 of the mature protein) correspond to the intracellular domain. As used herein, the term CD122 includes naturally occurring variants of the CD122 protein including the S57F and D365E (as numbered in accordance with the mature hCD122 protein). The consensus wild-type hCD122 nucleic acid and protein sequences may be found as Genbank accession numbers NM_000878 and NP_000869 respectively.

CD132 is a type 1 cytokine receptor and is shared by the receptor complexes for IL-4, IL-7, IL-9, IL-15, and IL-21, and is consequently referred in the literature as the “common” gamma chain. Human CD132 (hCD132) is expressed as a 369 amino acid pre-protein comprising a 22 amino acid N-terminal signal sequence. Amino acids 23-262 (amino acids 1-240 of the mature protein) correspond to the extracellular domain, amino acids 263-283 (amino acids 241-262 of the mature protein) correspond to the 21 amino acid transmembrane domain, and amino acids 284-369 (amino acids 262-347 of the mature protein) correspond to the intracellular domain. Human CD132 nucleic acid and protein sequences may be found as Genbank accession numbers. NM_000206 and NP_000197 respectively.

hIL2 possesses a Kd of approximately 10⁻⁹M with respect to the intermediate affinity CD122/CD132 (IL2βγ) receptor complex. The intermediate affinity receptor complex is predominantly expressed on resting T-cells and NK cells. In comparison, hIL2 possesses a Kd of approximately 10⁻¹¹M with respect to the high IL2 affinity receptor complex. Most cells, such as resting T cells, demonstrate low responsiveness to IL2 since they only express the CD122 and CD132 which have comparatively low affinity for IL2 relative to the CD25/CD122/CD132 high affinity receptor complex. The high affinity receptor complex is predominantly identified on activated lymphocytes which inducibly express CD25 and Treg cells that express CD25 constituitively.

In either the selected TIL or young TIL process, the expansion of TILs as currently practiced is performed in the presence of hIL2. While wt-hIL2 ability to broadly and potently activate and induce the proliferation of T cells makes wt-hIL2 attractive for use in TIL therapy, its use in TIL therapy presents significant issues both ex vivo and in vivo.

The ex vivo exposure TILs to high dose IL2 has been associated with terminal differentiation of the T cells. The degree of T-cell differentiation of the T cells following ex vivo stimulation procedures can affect the survival, proliferative capacity and efficacy of the TILs in vivo following reinfusion to the extent that other cytokines such as IL-15 or IL21 have proposed for use to avoid the effects of IL2 in the ex vivo preparation of TILs to avoid the effects of IL2 in the ex vivo preparation of TILs. Li, et al. (2010) J Immunol. 2010; 184: 452-465. Furthermore, it is desirable that the final TIL product to be administered be as enriched as possible for the tumor-specific TIL clones. The non-specific nature of hIL2 fails to provide selective support for the tumor antigen experienced T cell clones and it is possible that the most efficacious tumor antigen experienced T cell clones will be out-competed and diluted during the ex vivo expansion phase. Additionally, a prolonged contact with IL2 ex vivo can result in over-stimulation of the isolated T cells such that the T cells and driven to exhaustion such that a significant fraction of the T cells to be reimplanted in the subject are not in the optimal state for anti-tumor effectiveness.

In vivo, the supportive regimens involving the systemic administration of hIL2 are also associated with significant toxicity as well as mediation of autoimmunity and transplant rejection in addition to other side effects. The most prevalent side effects seen in arising from the use of IL2 supportive therapy following adoptive cell transfer (ACT) include chills, high fever, hypotension, oliguria, and edema due to the systemic inflammatory and capillary leak syndrome as well as reports of autoimmune phenomena such as vitiligo or uveitis.

Apart from IL2 mediated issues discussed above, other aspects of the current practice of TIL therapy present toxicity issues for the patient. In the current practice of TIL therapy, following ex vivo expansion, a TIL cell product contains approximately 10¹¹ to 10¹³ cells. This large dose of cells to the patient indicates the utility of preparative lymphodepleting preparative regimens prior to reinfusion of the TILs. These lymphodepleting preparative regimens are associated with additional toxicities such pancytopenia and febrile neutropenia and the supportive therapy with high dose IL2 following re-administration of the enriched TIL cell population.

Consequently, in the context of TIL therapy, there is a need in the art for agents which enable the selective expansion and activation of the tumor antigen-experienced T cells population ex vivo without driving the desired population of these tumor antigen experienced T cells toward differentiation and/or exhaustion, agents which provide support for the activated TIL cell product without significant systemic toxicity and agents which avoid (or minimize the need for) lymphodepletion prior to reinfusion of the TIL cell product.

### SUMMARY OF THE DISCLOSURE

The present disclosure provides compositions and methods for the use of αβhIL2 muteins that selectively stimulate the proliferation of antigen experienced T cells ex vivo and optionally in vivo.

The present disclosure provides method of use of αβhIL2 muteins for the activation and expansion of antigen experienced T cells in an isolated population of cells.

In some embodiments, the present disclosure is directed to the use of αβhIL2 muteins ex vivo to prepare a population of cells enriched for antigen experienced T cells and administering the population of cells to a subject.

In some embodiments, the present disclosure is directed to the use of an αβhIL2 mutein ex vivo to prepare a population of cells enriched for antigen experienced T cells and administering the population of cells to a subject.

In some embodiments, the present disclosure is directed to the use of αβhIL2 mutein ex vivo to prepare a population of cells enriched for antigen experienced T cells and administering the population of cells to a subject and administering to said subject a therapeutically effective amount of a αβhIL2 mutein (e.g., such that the administered population of cells proliferate and have a therapeutic effect).

In some embodiments, the present disclosure is directed to the use of αβhIL2 mutein ex vivo to prepare a polyclonal population of cells enriched for antigen experienced T cells and administering the population of cells to a subject and administering to said subject a therapeutically effective amount of a IL2 mutein of the present disclosure.

In some embodiments, the present disclosure is directed to the use of αβhIL2 mutein ex vivo to prepare a polyclonal population of cells enriched for tumor antigen experienced T cells and administering the population of cells to a subject and administering to said subject a therapeutically effective amount of an αβhIL2 mutein.

In some embodiments, the present disclosure is directed to the use of a first αβhIL2 mutein ex vivo to prepare a polyclonal population of cells enriched for tumor antigen experienced T cells and administering the population of cells to a subject and administering to said subject a therapeutically effective amount of a second αβhIL2 mutein, wherein the first αβhIL2 mutein and second αβhIL2 mutein are the same.

In some embodiments, the present disclosure is directed to the use of a first αβhIL2 mutein ex vivo to prepare a polyclonal population of cells enriched for tumor antigen experienced T cells and administering the population of cells to a subject and administering to said subject a therapeutically effective amount of a second αβhIL2 mutein of the present disclosure, wherein the first IL2 mutein of αβhIL2 mutein and second αβhIL2 mutein comprise the same amino acid sequence. In some embodiments, the present disclosure is directed to the use of a first IL2 mutein ex vivo to prepare a polyclonal population of cells enriched for tumor antigen experienced T cells and administering the population of cells to a subject and administering to said subject a therapeutically effective amount of a second biased hIL2 mutein, wherein the first αβhIL2 mutein and second αβhIL2 mutein comprise the same amino acid sequence but the second αβhIL2 mutein is modified to provide for extended half-life in vivo.

In some embodiments, the present disclosure is directed to the use of a first αβhIL2 mutein ex vivo to prepare a polyclonal population of cells enriched for tumor antigen experienced T cells and administering the population of cells to a subject and administering to said subject a therapeutically effective amount of a second a αβhIL2 mutein having reduced binding affinity for the extracellular domain of hCD132, wherein the first αβhIL2 mutein and second αβhIL2 mutein comprise different amino acid sequences.

In some embodiments, the present disclosure provides a method of use of a cell population enriched for antigen experienced T cells the method comprising the step of administering said cell population to a subject for the treatment of a disease, disorder or condition. In some embodiments, the present disclosure provides a method of use of a cell population enriched for antigen experienced T cells the method comprising the step of administering said cell population to a subject for the treatment of the disease, disorder or condition in combination with an αβhIL2 mutein.

In some embodiments, the present disclosure provides methods of treating a subject suffering from a disease, disorder or condition by obtaining a sample of a tissue (e.g., blood, tumor tissue) from said subject, isolating antigen experienced T cells from said sample of tissue, and contacting the isolated antigen experienced T cells ex vivo with an αβhIL2 mutein 2.

In some embodiments, the present disclosure provides methods of preparing a population of T cells comprising polyclonal antigen experienced T cells, the method comprising the steps of obtaining a sample of a tissue (e.g., blood, tumor tissue) from a subject suffering from a disease, disorder or condition, isolating antigen experienced T cells from said sample of tissue, and contacting the isolated antigen experienced T cells ex vivo with an αβhIL2 mutein.

In some embodiments, the present disclosure provides a population of T cells comprising a population of polyclonal antigen experienced T cells said population prepared by the method of. obtaining a sample of a tissue (e.g. blood, tumor tissue) from a subject suffering from a disease, disorder or condition; isolating antigen experienced T cells from said sample of tissue and contacting the isolated antigen experienced T cells ex vivo with an αβhIL2 mutein.

In some embodiments, the present disclosure provides methods of treating a subject suffering from a disease, disorder or condition by obtaining a sample of a tissue (e.g. blood, tumor tissue) from said subject, isolating antigen experienced T cells from said sample of tissue, contacting the isolated antigen experienced T cells ex vivo with an αβhIL2 mutein to provide a population of cells enriched for antigen experienced T cells, and administering said population of cells to the subject. In some embodiments, the tissue is a neoplasm. In some embodiments, the neoplasm is a solid tumor. In some embodiments, the tissue is blood.

In some embodiments, the present disclosure provides the use of αβhIL2 muteins in combination with adoptive cell therapy (e.g., TIL therapy) during either the ex vivo phase and/or in vivo phase.

In some embodiments, the present disclosure provides the use of αβhIL2 muteins in combination with TIL therapy during the ex vivo phase and the in vivo phase. In some embodiments, the present disclosure provides the use of IL2 muteins in combination with TIL therapy during either the ex vivo TIL expansion phase and the in vivo phase wherein the biased IL2 mutein used in the ex vivo TIL expansion phase is the same as the αβhIL2 mutein used in the in vivo phase. In some embodiments, present disclosure provides the use of αβhIL2 mutein in combination with TIL therapy during either the ex vivo phase and the in vivo phase wherein the αβhIL2 mutein used in the ex vivo TIL expansion phase is different from the αβhIL2 mutein used in the in vivo TIL support phase.

The desirable cell subpopulation of the isolated TILs are those cells which have recently been activated by exposure to tumor antigen in the presence of TCR signal. Contact with a tumor antigen and co-stimulation by TCR upregulates the expression of CD25 such that “antigen experienced” is correlated with the CD8+ CD25+ phenotype.

In some embodiments, the present disclosure provides a method of treating a subject suffering from a neoplastic disease, said method comprising the steps of
(a) isolating a tissue sample from the subject, the tissue sample comprising a population of TILs;
(b) contacting the isolated tissue sample of step (a) ex vivo with a quantity of an αβhIL2 mutein at a concentration sufficient to induce proliferation and activation of the TILs to generate an expanded cell population comprising activated TILs; and
(c) administering to the subject the expanded cell population comprising activated TILs from step (b).

In some embodiments, the present disclosure provides a method of treating a subject suffering from a neoplastic disease, said method comprising the steps of
(a) isolating a tissue sample from the subject, the tissue sample comprising a population of antigen activated T-cells;
(b) contacting the tissue sample of step (a) ex vivo with a quantity of a first αβhIL2 mutein sufficient to induce proliferation of antigen activated T-cells to generate an expanded cell population comprising antigen activated T-cells;
(c) administering to the subject a population of the antigen activated T-cell cells from the expanded cell population of step (b); and
(d) administering to the subject a therapeutically effective amount of a second αβhIL2 mutein,
   wherein the first and second αβhIL2 muteins are the same or different.

In some embodiments, the present disclosure provides a method of treating a subject suffering from a neoplastic disease, said method comprising the steps of
(a) administering to the subject a therapeutically effective amount of a first αβhIL2 mutein;
(b) Isolating a tissue sample from the subject, the tissue sample comprising a population of antigen activated T-cells;
(c) contacting the tissue sample of step (b) ex vivo with a quantity of a second αβhIL2 mutein sufficient to induce proliferation of antigen activated T-cells to generate an expanded cell population comprising antigen activated T-cells; and
(d) administering to the subject a population of the antigen activated T-cell cells from the expanded cell population of step (c).

In some embodiments, the present disclosure provides a method of treating a subject suffering from a neoplastic disease, said method comprising the steps of
(a) administering to the subject a therapeutically effective amount of a first αβhIL2 mutein;
(b) isolating a tissue sample from the subject, the tissue sample comprising a population of antigen activated T-cells;
(c) contacting the tissue sample of step (b) ex vivo with a quantity of a second αβhIL2 mutein sufficient to induce proliferation of antigen activated T-cells to generate an expanded cell population comprising antigen activated T-cells; and
(d) administering to the subject a population of the antigen activated T-cell cells from the expanded cell population of step (c); and
(e) administering to the subject a therapeutically effective amount of a third αβhIL2 mutein,
   wherein the first, second and third αβhIL2 muteins are the same; the first and third αβhIL2 muteins are the same; the first and second αβhIL2 muteins are the same, or each of the first, second and third αβhIL2 muteins are different αβhIL2 muteins.

In some embodiments, the present disclosure provides a method of treating a subject suffering from a neoplastic disease, said method comprising the steps of
(a) isolating a tissue sample from the subject, the tissue sample comprising a population of antigen activated T-cells;
(b) applying an ex vivo cell selection process to the isolated tissue sample of step (a) to generate a subpopulation of antigen activated T-cells enriched for antigen activated T-cells possessing activation marker antigens;
(c) expanding the subpopulation of antigen activated T-cells enriched for one or more marker antigens generated from step (b) by contacting the subpopulation of antigen activated T-cells enriched for one or more marker antigens ex vivo with a quantity of a second αβhIL2 mutein sufficient to induce proliferation of antigen activated T-cells, or optionally for a period of time sufficient to expand a quantity of the antigen activated T-cells possessing one or more marker antigens, to generate an expanded cell population comprising antigen activated T-cells enriched for one or more marker antigens; and
(d) administering to the subject a quantity of antigen activated T-cells enriched for activation marker antigens from the expanded cell population comprising antigen activated T-cells enriched for activation marker antigens of step (c).

In some embodiments, the present disclosure provides a method of treating a subject suffering from a neoplastic disease, said method comprising the steps of:
(a) isolating a tissue sample from the subject, the tissue sample comprising a population of antigen activated T-cells;
(b) applying an ex vivo cell selection process to the isolated tissue sample of step (a) to generate a subpopulation of antigen activated T-cells enriched for antigen activated T-cells possessing one or more marker antigens;
(c) expanding the subpopulation of antigen activated T-cells enriched for one or more marker antigens generated from step (b) by contacting the subpopulation of antigen activated T-cells enriched for one or more marker antigens ex vivo with a quantity of a second αβhIL2 mutein sufficient to induce proliferation of antigen activated T-cells, or optionally for a period of time sufficient to expand a quantity of the antigen activated T-cells possessing one or more marker antigens, to generate an expanded cell population comprising antigen activated T-cells enriched for one or more marker antigens; and
(d) administering to the subject a quantity of antigen activated T-cells enriched for one or more marker antigens from the expanded cell population comprising antigen activated T-cells enriched for activation marker antigens of step (c); and
(e) administering to the subject a therapeutically effective amount of a second αβhIL2 mutein,
   wherein the first and second αβhIL2 muteins are the same or different.

In some embodiments, the present disclosure provides a method of treating a subject suffering from a neoplastic disease, said method comprising the steps of:
(a) administering to the subject a therapeutically effective amount of a first αβhIL2 mutein;
(b) isolating a tissue sample from the subject, the tissue sample comprising a population of antigen activated T-cells;
(c) applying an ex vivo cell selection process to the isolated tissue sample of step (a) to generate a subpopulation of antigen activated T-cells enriched for antigen activated T-cells possessing activation marker antigens;
(d) expanding the subpopulation of antigen activated T-cells enriched for one or more marker antigens generated from step (c) by contacting the subpopulation of antigen activated T-cells enriched for one or more marker antigens ex vivo with a quantity of a second αβhIL2 mutein sufficient to induce proliferation of antigen activated T-cells, or optionally for a period of time sufficient to expand a quantity of the antigen activated T-cells possessing one or more marker antigens, to generate an expanded cell population comprising antigen activated T-cells enriched for one or more marker antigens; and
(e) administering to the subject a quantity of antigen activated T-cells enriched for activation marker antigens from the expanded cell population comprising antigen activated T-cells enriched for activation marker antigens of step (d), wherein the first and second αβhIL2 muteins are the same or different.

In some embodiments, the present disclosure provides a method of treating a subject suffering from a neoplastic disease, said method comprising the steps of:
(a) administering to the subject a therapeutically effective amount of a first αβhIL2 mutein;
(b) isolating a tissue sample from the subject, the tissue sample comprising a population of antigen activated T-cells;
(c) applying an ex vivo cell selection process to the isolated tissue sample of step (a) to generate a subpopulation of antigen activated T-cells enriched for antigen activated T-cells possessing activation marker antigens;
(d) expanding the subpopulation of antigen activated T-cells enriched for one or more marker antigens generated from step (c) by contacting the subpopulation of antigen activated T-cells enriched for one or more marker antigens ex vivo with a quantity of a second αβhIL2 mutein sufficient to induce proliferation of antigen activated T-cells, or optionally for a period of time sufficient to expand a quantity of the antigen activated T-cells possessing one or more marker antigens, to generate an expanded cell population comprising antigen activated T-cells enriched for one or more marker antigens; and
(e) administering to the subject a quantity of antigen activated T-cells enriched for activation marker antigens from the expanded cell population comprising antigen activated T-cells enriched for one or more marker antigens of step (d), (f) administering to the subject a therapeutically effective amount of a third αβhIL2 mutein,
   wherein the first, second and third αβhIL2 muteins are the same; the first and third αβhIL2 muteins are the same; the first and second αβhIL2 muteins are the same, or each of the first, second and third αβhIL2 muteins are different αβhIL2 muteins.

In some embodiments, the present disclosure provides a method of treating a subject suffering from a neoplastic disease, said method comprising the steps of:
(a) isolating a tissue sample from the subject, the tissue sample comprising a population of antigen activated T-cells;
(b) contacting the tissue sample of step (a) ex vivo with a quantity of an αβhIL2 mutein sufficient to induce proliferation of antigen activated T-cells to generate an expanded cell population comprising antigen activated T-cells;
(c) contacting the expanded cell population comprising antigen activated T-cells of step (b) with a T-cell activation agent; and
(d) administering a population of the antigen activated T-cell cells from the expanded cell population of step (c) to the subject.

In some embodiments, the present disclosure provides a method of treating a subject suffering from a neoplastic disease, said method comprising the steps of
(a) isolating a tissue sample from the subject, the tissue sample comprising a population of antigen activated T-cells;
(b) contacting the tissue sample of step (a) ex vivo with a quantity of a first αβhIL2 mutein sufficient to induce proliferation of antigen activated T-cells to generate an expanded cell population comprising antigen activated T-cells;
(c) contacting the expanded cell population comprising antigen activated T-cells of step (b) with a T-cell activation agent; and
(d) administering to the subject a population of the antigen activated T-cells from the expanded cell population of step (c); and
(e) administering to the subject a therapeutically effective amount of a second αβhIL2 mutein,
   wherein the first and second αβhIL2 muteins are the same or different.

In some embodiments, the present disclosure provides a method of treating a subject suffering from a neoplastic disease, said method comprising the steps of
(a) administering to the subject a therapeutically effective amount of a first αβhIL2 mutein;
(b) isolating a tissue sample from the subject, the tissue sample comprising a population of antigen activated T-cells;
(c) contacting the tissue sample of step (b) ex vivo with a quantity of a second αβhIL2 mutein sufficient to induce proliferation of antigen activated T-cells to generate an expanded cell population comprising antigen activated T-cells;
(d) contacting the expanded cell population comprising antigen activated T-cells of step (c) with a T-cell activation agent; and
(e) administering to the subject a population of the antigen activated T-cells from the expanded cell population of step (d).

In some embodiments, the present disclosure provides a method of treating a subject suffering from a neoplastic disease, said method comprising the steps of
(a) administering to the subject a therapeutically effective amount of a first αβhIL2 mutein;
(b) isolating a tissue sample from the subject, the tissue sample comprising a population of antigen activated T-cells;
(c) contacting the tissue sample of step (b) ex vivo with a quantity of a second αβhIL2 mutein sufficient to induce proliferation of antigen activated T-cells to generate an expanded cell population comprising antigen activated T-cells; and
(d) contacting the expanded cell population comprising antigen activated T-cells of step (c) with a T-cell activation agent; and
(e) administering to the subject a population of the antigen activated T-cell cells from the expanded cell population of step (d); and
(f) administering to the subject a therapeutically effective amount of a third αβhIL2 mutein,
   wherein the first, second and third αβhIL2 muteins are the same; the first and third αβhIL2 muteins are the same; the first and second αβhIL2 muteins are the same, or each of the first, second and third αβhIL2 muteins are different αβhIL2 muteins.

In some embodiments, the present disclosure provides a method of treating a subject suffering from a neoplastic disease, said method comprising the steps of
(a) isolating a tissue sample from the subject, the tissue sample comprising a population of antigen activated T-cells;
(b) applying an ex vivo cell selection process to the isolated tissue sample of step (a) to generate a subpopulation of antigen activated T-cells enriched for antigen activated T-cells possessing activation marker antigens;
(c) expanding the subpopulation of antigen activated T-cells enriched for one or more marker antigens generated from step (b) by contacting the subpopulation of antigen activated T-cells enriched for one or more marker antigens ex vivo with a quantity of a second αβhIL2 mutein sufficient to induce proliferation of antigen activated T-cells, or optionally for a period of time sufficient to expand a quantity of the antigen activated T-cells possessing one or more marker antigens, to generate an expanded cell population comprising antigen activated T-cells enriched for one or more marker antigens; and
(d) contacting the expanded cell population comprising antigen activated T-cells of step (c) with a T-cell activation agent; and
(e) administering to the subject a quantity of antigen activated T-cells enriched for activation marker antigens from the expanded cell population comprising antigen activated T-cells enriched for activation marker antigens of step (d).

In some embodiments, the present disclosure provides a method of treating a subject suffering from a neoplastic disease, said method comprising the steps of:
(a) isolating a tissue sample from the subject, the tissue sample comprising a population of antigen activated T-cells;
(b) applying an ex vivo cell selection process to the isolated tissue sample of step (a) to generate a subpopulation of antigen activated T-cells enriched for antigen activated T-cells possessing one or more marker antigens;
(c) expanding the subpopulation of antigen activated T-cells enriched for one or more marker antigens generated from step (b) by contacting the subpopulation of antigen activated T-cells enriched for one or more marker antigens ex vivo with a quantity of a second αβhIL2 mutein sufficient to induce proliferation of antigen activated T-cells, or optionally for a period of time sufficient to expand quantity of the antigen activated T-cells possessing one or more marker antigens, to generate an expanded cell population comprising antigen activated T-cells enriched for one or more marker antigens; and
(d) contacting the expanded cell population comprising antigen activated T-cells of step (c) with a T-cell activation agent; and
(e) administering to the subject a quantity of antigen activated T-cells enriched for one or more marker antigens from the expanded cell population comprising antigen activated T-cells enriched for activation marker antigens of step (d); and
(f) administering to the subject a therapeutically effective amount of a second αβhIL2 mutein,
   wherein the first and second αβhIL2 muteins are the same or different.

In some embodiments, the present disclosure provides a method of treating a subject suffering from a neoplastic disease, said method comprising the steps of:
a. administering to the subject a therapeutically effective amount of a first αβhIL2 mutein;
b. isolating a tissue sample from the subject, the tissue sample comprising a population of antigen activated T-cells;
c. applying an ex vivo cell selection process to the isolated tissue sample of step (a) to generate a subpopulation of antigen activated T-cells enriched for antigen activated T-cells possessing activation marker antigens;
d. expanding the subpopulation of antigen activated T-cells enriched for one or more marker antigens generated from step (c) by contacting the subpopulation of antigen activated T-cells enriched for one or more marker antigens ex vivo with a quantity of a second αβhIL2 mutein sufficient to induce proliferation of antigen activated T-cells, optionally for a period of time sufficient to expand a quantity of the antigen activated T-cells possessing one or more marker antigens, to generate an expanded cell population comprising antigen activated T-cells enriched for one or more marker antigens;
e. contacting the expanded cell population comprising antigen activated T-cells of step (d) with a T-cell activation agent; and
f. administering to the subject a quantity of antigen activated T-cells enriched for activation marker antigens from the expanded cell population comprising antigen activated T-cells enriched for activation marker antigens of step (e), wherein the first and second αβhIL2 muteins are the same or different.

In some embodiments, the present disclosure provides a method of treating a subject suffering from a neoplastic disease, said method comprising the steps of:
a. administering to the subject a therapeutically effective amount of a first αβhIL2 mutein;
b. isolating a tissue sample from the subject, the tissue sample comprising a population of antigen activated T-cells;
c. applying an ex vivo cell selection process to the isolated tissue sample of step (a) to generate a subpopulation of antigen activated T-cells enriched for antigen activated T-cells possessing activation marker antigens;
d. expanding the subpopulation of antigen activated T-cells enriched for one or more marker antigens generated from step (c) by contacting the subpopulation of antigen activated T-cells enriched for one or more marker antigens ex vivo with a quantity of a second αβhIL2 mutein sufficient to induce proliferation of antigen activated T-cells, optionally for a period of time sufficient to expand a quantity of the antigen activated T-cells possessing one or more marker antigens, to generate an expanded cell population comprising antigen activated T-cells enriched for one or more marker antigens; and
e. contacting the expanded cell population comprising antigen activated T-cells of step (d) with a T-cell activation agent; and
f. administering to the subject a quantity of antigen activated T-cells enriched for activation marker antigens from the expanded cell population comprising antigen activated T-cells enriched for one or more marker antigens of step (e),
g. administering to the subject a therapeutically effective amount of a third αβhIL2 mutein,
   wherein the first, second and third αβhIL2 muteins are the same; the first and third αβhIL2 muteins are the same; the first and second αβhIL2 muteins are the same, or each of the first, second and third αβhIL2 muteins are different αβhIL2 muteins.

The present disclosure provides the conduct of any of the foregoing methods wherein the tissue sample is selected from the group consisting of blood and solid tumor tissue

The present disclosure provides the conduct of any of the foregoing methods wherein the subject is treated with a lymphodepleting regimen prior to the administration of the quantity of antigen activated T-cells to the subject. The present disclosure provides the conduct of any of the foregoing methods wherein the αβhIL2 mutein is an IL2 mutein having at least 90% sequence identity to wt-hIL2 (SEQ ID NO:4), the αβhIL2 mutein comprising an amino acid substitution at position 18, 22 or 126 numbered in accordance with wt-hIL2 (SEQ ID NO:4). The present disclosure provides the conduct of any of the foregoing methods wherein the αβhIL2 mutein comprises amino acid substitutions at one or more positions selected from R18, Q22 and/or Q126 numbered in accordance with the mature wild-type human IL2 (SEQ ID NO: 4). The present disclosure provides the conduct of any of the foregoing methods wherein the αβhIL2 mutein comprises amino acid substitutions at positions R18E, Q22K and Q126K numbered in accordance with the mature wild-type human IL2 (SEQ ID NO: 4). In some embodiments, αβhIL2 mutein comprises an amino acid substitution is selected from the group consisting of L18R, L18G, L18M, L18F, L18E, L18H, L18W, L18K, L18Q, L18S, L18V, L18I, L18Y, L18H, L18D, L18N, L18T, Q22F, Q22E, Q22G, Q22A, Q22L, Q22M, Q22F, Q22W, Q22K, Q22S, Q22V, Q22I, Q22Y, Q22H, Q22R, Q22N, Q22D, Q22T, Q22F, Q126H, Q126M, Q126K, Q126C, Q126D, Q126E, Q126G, Q126I, Q126R, Q126S, and Q126T. In some embodiments, αβhIL2 mutein comprises the αβhIL2 mutein is an IL2 mutein having at least 90% sequence identity to wt-hIL2 (SEQ ID NO:4), the αβhIL2 mutein comprising three amino acid substitutions at position 18, 22 and 126 numbered in accordance with wt-hIL2 (SEQ ID NO:4). In some embodiments, αβhIL2 mutein comprises amino acid substitutions at positions 18, 22 and 126 wherein: (a) the amino acid substitution at position 18 of the αβhIL2 mutein is selected from the group consisting of L18R, L18G, L18M, L18F, L18E, L18H, L18W, L18K, L18Q, L18S, L18V, L18I, L18Y, L18H, L18D, L18N and L18T; (b) the amino acid substitution at position 22 of the αβhIL2 mutein is selected from the group consisting of Q22F, Q22E, Q22G, Q22A, Q22L, Q22M, Q22F, Q22W, Q22K, Q22S, Q22V, Q22I, Q22Y, Q22H, Q22R, Q22N, Q22D, Q22T, and Q22F; and (c) the amino acid substitution at position 126 of the of the αβhIL2 mutein is selected from the group consisting of Q126H, Q126M, Q126K, Q126C, Q126D, Q126E, Q126G, Q126I, Q126R, Q126S, or Q126T. In some embodiments, αβhIL2 mutein comprises a set of mutations selected from the group consisting of the following sets of mutations: L18R, Q22E, and Q126K; L18R, Q22E, and Q126H; L18R, Q22E and Q126M; L18R, Q22E Q126T; L18R; Q22E; V91K; V91R; Q126H; L18R, and Q126H; Q22E, and Q126H; L18G, Q22E and Q126H; L18A, Q22E and Q126H; L18M, Q22E and Q126H; L18F, Q22E and Q126H; L18W, Q22E and Q126H; L18K, Q22E and Q126H; L18Q, Q22E and Q126H; L18E, Q22E and Q126H; L18S, Q22E and Q126H; L18V, Q22E and Q126H; L18I, Q22E and Q126H; L18Y, Q22E and Q126H; L18H, Q22E and Q126H; L18N, Q22E and Q126H; L18D, Q22E and Q126H; L18T, Q22E and Q126H; L18R, Q22G and Q126H; L18R, Q22A and Q126H; L18R, Q22L and Q126H; L18R, Q22M and Q126H; L18R, Q22F and Q126H; L18R, Q22W and Q126H; L18R, Q22K and Q126H; L18R, Q22S and Q126H; L18R, Q22V and Q126H; L18R, Q22I and Q126H; L18R Q22Y and Q126H; L18R Q22H and Q126H; L18R Q22R and Q126H; L18R Q22N and Q126H; L18R Q22D and Q126H; and L18R Q22T and Q126H. In some embodiments, the αβhIL2 mutein comprises a deletion of 1, 2, 3, 4, 5, 6, 7, 8, or 9 N-terminal amino acids. In some embodiments, the αβhIL2 mutein comprises a deletion of 1, 2, or 3 N-terminal amino acids. In some embodiments, the αβhIL2 mutein comprises a deletion of the N-terminal alanine amino acid (des-Alal). In some embodiments, the αβhIL2 mutein modified to extend its duration of action in vivo.

The present disclosure provides the conduct of any of the foregoing methods wherein the one or more marker antigens is selected from one or more antigens selected from cell type antigens and activation antigens. In some embodiments one or more antigens selected from CD3, CD4, CD8, CD11a, CD11b, CD11c, CD14, CD16, CD19, CD25, CD27, CD28, CD38 CD45RA, CD45RO, CD58, CD61, CD62L, CD66b, CD69, CD103, CD122, CD127, CD197, CD279, D62L, CD69, FoxP3, PD-1, D62L, CCR4, CCR5, CCR6(CD196), CCR7, CCR10, CXCR3, CTLA4, PD1, PDL1, TCRγδ, TCRVα24, TCRVβ1, HLA-DR, Ki67, T-bet, GATA-3, PU.1, RORγt, AHR, FOXO4, and FOXP3

The present disclosure provides the conduct of any of the foregoing methods wherein the step of contacting the isolated population of cells with an αβhIL2 mutein is practiced in combination with one or more additional T-cell activation agent. In some embodiments, the T cell activation agent is selected from cytokines, growth factors, antibodies to T-cell activation antigens (e.g., anti-CD3 antibodies, anti-CD137 antibodies). Examples of T cell activation agents include CD3/CD28 beads.

The present disclosure provides the conduct of any of the foregoing methods wherein, the isolated T cell population is contacted with a recombinant vector comprising a nucleic acid sequence encoding an engineered receptor which is selectively activated in response to the administration of a cognate ligand which binds to the extracellular domain of the engineered receptor and results in intracellular signaling in the T cells expressing the engineered receptor.

The present disclosure provides the conduct of any of the foregoing methods wherein the method is practiced in combination with the administration of a supplementary agent to the subject. In some embodiments, the supplementary agent is selected from the group consisting of chemotherapeutic agents, antibodies, immune checkpoint modulators and physical methods. In some embodiments, the immune checkpoint modulator is an anti-PD-1 or anti-PD-L1 antibody. In some embodiments, the supplementary agent is an antibody selected from the group consisting of [fam]-trastuzumab deruxtecan, enfortumab vedotin, polatuzumab vedotin, cemiplimab, moxetumomab pasudotox, mogamuizumab, tildrakizumab,ibalizumab, durvalumab, inotuzumab, ozogamicin, avelumab, atezolizumab, olaratumab, ixekizumab, aratumumab, elotuzumab, necitumumab, dinutuximab, nivolumab, blinatumomab, pembrolizumab, ramucirumab, siltuximab, obinutuzumab, ado-trastuzumab emtansine, pertuzumab, brentuximab vedotin, ipilimumab, ofatumumab, certolizumab pegol, catumaxomab, panitumumab, bevacizumab, cetuximab, tositumomab-I131, ibritumomab tiuxetan, gemtuzumab, ozogamicin, trastuzumab, infliximab, rituximab, and edrecolomab.

The present disclosure provides the conduct of any of the foregoing methods wherein the neoplastic disease, disorder or condition is selected from the group consisting of. adenomas, fibromas, hemangiomas, hyperplasia, atypia, metaplasia, dysplasia, carcinomas, leukemias, breast cancers, sarcomas, leukemias, lymphomas, genitourinary cancers, ovarian cancers, urethral cancers, bladder cancers, prostate cancers, gastrointestinal cancers, colon cancers, esophageal cancers, stomach cancers, lung cancers; myelomas; pancreatic cancers; liver cancers; kidney cancers; endocrine cancers; skin cancers; gliomas, neuroblastomas, astrocytomas, myelodysplastic disorders; cervical carcinoma-in-situ; intestinal polyposes; oral leukoplakias; histiocytoses, hyperprofroliferative scars including keloid scars, respiratory system carcinomas, gastrointestinal system carcinomas, genitourinary system carcinomas, testicular carcinomas, breast carcinomas, prostatic carcinomas, endocrine system carcinomas, melanomas, adenocarcinomas, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage, promyeloid leukemia (APML), acute myelogenous leukemia (AML) and chronic myelogenous leukemia (CML), precursor lymphoid neoplasms, mature B-cell neoplasms, mature T-cell neoplasms, Hodgkin's Lymphoma, and immunodeficiency-associated lymphoproliferative disorders, lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). erythroblastic leukemia and acute megakaryoblastic leukemia, malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), and Hodgkin's disease.

In some embodiments, the present disclosure provides a method of treating a subject suffering from a neoplastic disease, said method comprising the steps of:
(a) isolating a tissue sample from the subject, the tissue sample comprising a population of TILs;
(b) contacting the isolated tissue sample of step (a) ex vivo with a quantity of an αβhIL2 mutein at a concentration sufficient to induce proliferation and activation of the TILs to generate an expanded cell population comprising activated TILs;
(c) contacting the expanded cell population with a recombinant vector comprising a nucleic acid sequence encoding an engineered receptor which is selectively activated in response to the administration of a cognate ligand which binds to the extracellular domain of the engineered receptor and results in intracellular signaling in the T cells expressing the engineered receptor
(d) administering to the subject the expanded cell population comprising activated TILs from step (b).
(e) administering to the subject a therapeutically effective amount of a cognate ligand for the engineered receptor.

In some embodiments, the present disclosure provides a method of treating a subject suffering from a neoplastic disease, said method comprising the steps of
(a) isolating a tissue sample from the subject, the tissue sample comprising a population of T cells;
(b) applying an ex vivo cell selection process to the isolated tissue sample of step (a) to generate a population of T-cells enriched for one or more marker antigens;
(c) contacting population of T-cells from step (b) ex vivo with a quantity of an αβhIL2 mutein at a concentration sufficient to induce proliferation and activation of T cells;
(d) contacting the population of T cells from step (c) with a recombinant vector comprising a nucleic acid sequence encoding an engineered receptor which is selectively activated in response to the administration of a cognate ligand which binds to the extracellular domain of the engineered receptor and results in intracellular signaling in the T cells expressing the engineered receptor; (e) administering to the subject the expanded cell population from step (d).
(e) administering to the subject a therapeutically effective amount of a cognate ligand for the engineered receptor.

In some embodiments of the practice of the foregoing methods, prior to the administration of the cell population to the subject the subject is treated with a lymphodepleting regimen. In some embodiments of the practice of the foregoing methods, prior to the administration of the cell population to the subject the cell population is contacted with a T-cell activation agent. In some embodiments of the practice of the foregoing methods, prior to the administration of the cell population engineered to express the engineered receptor, the subject is pretreated in vivo with a therapeutically effective amount of an αβhIL2 mutein. In some embodiments of the practice of the foregoing methods, wherein the cell population engineered to express the engineered receptor, the engineered receptor is an hCD122 comprising at least one amino acid substitution at position selected from positions 133 or 134 numbered in accordance with SEQ ID NO:2.

In some embodiments of the practice of the foregoing methods, the cell population engineered to express the engineered receptor, the engineered receptor is an hCD122 comprising amino acid substitutions at positions 133 and 134. In some embodiments, the engineered receptor is an hCD122 comprising amino acid substitutions H133D and Y134F.

In some embodiments of the practice of the foregoing methods, wherein the cell cell population engineered to express the engineered receptor, the cognate ligand is a hIL2 variant that selectively binds an hCD122 comprising at least one amino acid substitution at position selected from positions 133 or 134 numbered in accordance with SEQ ID NO:2. In some embodiments, the cognate ligand is an hIL2 variant comprising one or more amino acid substitutions at positions 15, 16, 19, 20, 22, 23, 51 or 81 numbered in accordance with wt hIL2 (SEQ ID NO: 4) wherein: the amino acid substitution at position 15 selected from E15S, E15T, E15Q, or E15H; the amino acid substitution at position 16 is H16Q; the amino acid substitution at position 19 is selected from L19V or L191; the amino acid substitution at position 20 is selected from D20T, D20S, D20L or D20M; the amino acid substitution at position 22 is selected from Q22K, Q22N; the amino acid substitution at position 23 is selected from M23L, M23S, M23V, M23A, or M23T; and the amino acid substitution at position 81 is selected from R81D and R81Y. In some embodiments, the cognate ligand is an hIL2 variant comprising an amino acid substitution at position 15 selected from E15S, E15T, E15Q, or E15H; an amino acid substitution at position 16 is H16Q; an amino acid substitution at position 19 selected from L19V or L191; an amino acid substitution at position 20 selected from D20T, D20S, D20L or D20M; an amino acid substitution at position 22 selected from Q22K, Q22N; an amino acid substitution at position 23 selected from M23L, M23S, M23V, M23A, or M23T. In some embodiments, the cognate ligand is an hIL2 variant comprising the amino acid substitutions E15S, H16Q, L19V, D20L; Q22K and M23A, optionally further comprising a deletion of the N-terminal alanine residue. In some embodiments, the cognate ligand is modified to extend its duration of action in vivo. In some embodiments, the modification to extend the duration of action in vivo is PEGylation. In some embodiments, the cognate ligand is an hIL2 mutein is modified by the N-terminal addition of 40 kDa branched PEG molecule.

The present disclosure further provides a cell product enriched for tumor antigen experienced T cells, the cell product prepared by a process comprising the steps of:
(a) isolating a tissue sample from the subject, the tissue sample comprising a population of TILs;
(b) contacting the tissue sample of step (a) ex vivo with a quantity of an αβhIL2 mutein at a concentration sufficient to induce proliferation and activation of the TILs to generate an expanded cell population comprising activated TILs.

The present disclosure further provides a cell product enriched for tumor antigen experienced T cells, the cell product prepared by a process comprising the steps of:
(a) isolating a tissue sample from the subject, the tissue sample comprising a population of antigen activated T-cells;
(b) applying an ex vivo cell selection process to the isolated tissue sample of step (a) to generate a subpopulation of antigen activated T-cells enriched for antigen activated T-cells possessing one or more marker antigens;
(c)expanding the subpopulation of antigen activated T-cells enriched for one or more marker antigens generated from step (b) by contacting the subpopulation of antigen activated T-cells enriched for one or more marker antigens ex vivo with a quantity of a second αβhIL2 mutein sufficient to induce proliferation of antigen activated T-cells to generate an expanded cell population comprising antigen activated T-cells enriched for one or more marker antigens; and
(d) contacting the expanded cell population comprising antigen activated T-cells of step (c) with a T-cell activation agent.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 provides a graphical presentation of the data representing the percentage of CD8+ T cells that express IFNg (y-axis) in response the indicated test agent.

FIG. 2 provides a graphical presentation of the data representing the percentage of CD8+ T cells that express IFNg (y-axis) in response the indicated test agent.

FIG. 3 provides a graphical illustration the levels of in vivo STAT5 phosphorylation in a non-human primate of CD8+ T cells expressing various levels of CD25 and/or CD122 in response to increasing doses of a PEGylated αβhIL2 mutein. The level of pSTAT5 as determined by mean fluorescent intensity (MFI) in CD8+ T cells is presented on the y-axis. The figure legend indicates the different cell populations and symbols for the different dose levels and corresponding graphical symbols. The dose of the PEGylated αβhIL2 mutein (in nanogramsml) is present on the x-axis. These data illustrate that the PEGylated abhIL2 mutein selectively activates T cells expressing CD25 and that such activation is dependent on the presence of CD122.

FIG. 4 provides graphical representation of the levels of STAT5 phosphorylation in a CD25^{pos} and CD25^{neg} CD8+ T cells in a non-human primate in response to two different doses (250 μg/kg and 20 μg/kg) of PEGylated αβhIL2 mutein. The percentage of STAT5 positive cells is presented on the y-axis. The figure legend indicates the different cell populations and symbols for the different dose levels and corresponding graphical symbols. The time course of the experiments in days is presented on the x-axis.

FIG. 5 provides a graphical representation of data generated in a non-human primate treated with a PEGylated αβhIL2 mutein illustrating that the PEGylated αβhIL2 mutein induces the selective proliferation of CD25+ CD8+ T cells in response at two dose levels (250 μg/kg and 20 μg/kg). The percentage of K167+ CD8+ T cells is presented on the y-axis. The figure legend indicates the different cell populations and symbols for the different dose levels and corresponding graphical symbols.

FIG. 6 provides a graphical representation of data generated in a non-human primate treated with a PEGylated non-α-hIL2 mutein. The percentage of K167+ CD8+ T cells is presented on the y-axis. The figure legend indicates the different cell populations and corresponding graphical symbols. The various time points of evaluation are presented on the x-axis.

FIG. 7 provides a graphical representation of data generated in a non-human primate treated with a PEGylated αβhIL2 mutein. The level of IL2 mutein species observed is the serum of the primate (in nanograms/ml) is presented on the y-axix. The time course of the study is presented on the x-axis. The figure legend indicates the different treatment conditions and corresponding graphical symbols.

FIG. 8 provides a graphical representation of a time course study generated in a non-human primate treated with PEGylated non-α-hIL2 mutein versus PEGylated αβhIL2. The y-axis provides the level of pSTAT5+ in CD8+ CD25+ cells. The figure legend indicates the different treatment conditions and corresponding graphical symbols. The time course of the study is presented on the x-axis.

FIG. 9 illustrates the anti-tumor efficacy of the PEGylated αβ(REH) mIL2 in the treatment of an MC38 tumor in mice. FIG. 9, Panel A provides an illustration of the study design indicating the time of tumor implant and the timeline (in days) and the time points of the administration of the various PEGylated IL2 species. of FIG. 9, Panel B provides graphical presentation the estimated tumor volume (y-axis) with respect to time (x-axis) over the course of the study (29 days following implantation of the MC38 tumor cells). The figure legend indicates the different treatment conditions and corresponding graphical symbols. CR is an abbreviation for complete response. FIG. 9, Panel C provides a graphical presentation of MC38 tumor weights (y-axis) in response to various treatments (x-axis). The legend indicates the different treatment conditions and corresponding graphical symbols used in Panels B and C.

FIG. 10 is a summary of results of the evaluation of multiple parameters in response to various IL2 molecules. FIG. 10, Panel A provides an illustration of the study design indicating the time of tumor implant and the timeline (in days) and the time points of administration of the various PEGylated IL2 species. TILs were isolated from the tumor on day 18, sorted for CD25 expression and exposed to ex-vivo to MC38 tumor cells. FIG. 10, Panel B provides results of FACS sorting the fraction of CD8+ cells is represented on the y-axis while the fraction of CD25+ cells represented on the x-axis. The rectangle indicates those cells which represent CD25+ TILs. FIG. 10, Panels C, D, and E the y-axis provides the levels of IFNy (Panel C), GM-CSF (Panel D), and TNFα (Panel E) in picograms/ml (pg/ml) in CD25+ and CD25− CD8+ T cells isolated from the tumors of the MC38 injected mice. The figure legend indicates the different treatment conditions and corresponding graphical symbols used.

FIG. 11 provides data evaluating toxicity parameters of the IL2 muteins in a non-human primate. In FIG. 11, Panels A-F provide microscopic images of lung tissue derived from non-human primates treated with PBS control (Panel A), wt-hIL2 (Panel B), one dose of the non- a-IL-2-PEG (Panel C) dose; two dose of the non- a-IL-2-PEG (Panel D), αβhIL2-PEG mutein at the 20 μg/kg dose (low dose or “LD”, Panel I legend) in Panel E and αβhIL2-PEG mutein at the 250 μg/kg dose (high dose or “HD”, Panel I legend) in Panel F. FIG. 11, Panel G provides data in relation to the percent of CD25+ CD8+ T cells that are phospho-STAT5 positive (y-axis) and the time course of the experiment (x-axis). The figure legend indicates the different treatment conditions and corresponding graphical symbols. FIG. 11, Panel H provides data in relation to concentration of FoxP3+ cells per square millimeter observed in the lungs of animals treated with each of the test agents. The figure legend indicates the different treatment conditions and corresponding graphical symbols. of CD25. FIG. 11, Panel H provides data of the relative lung weights of the animals (y-axis) normalized with respect to the untreated control animal in response to various test agents. The figure legend indicates the different test agents and corresponding graphical symbols. As previously noted, LD=low dose and HD=high dose.

FIG. 12 of the attached drawings provides a graphical representation of pSTAT5 levels as measured in NKL cells treated with 293T transfection supernatant containing the indicated IL2 muteins (and controls) as described in the for a variety of human IL2 muteins. The vertical axis represents the level of IL2 activity as determined by the maximum level of induction of phosphor-STAT 5 in the and each bar indicates the level of activity of the particular IL2 peptide evaluated associated with the construct as identified by a three letter abbreviation corresponding to the amino acids at positions 18, 22, and 126 of the hIL2 mutein numbered in accordance with wildtype hIL2 with of the with the exception of the V91K mutein which has a valine to lysine substitution at position 91.

FIG. 13 of the attached drawings provides comparative pSTAT5 activity in CD25 positive (CD25+) and CD25 negative (CD25-) YT cells treated with 293T transfection supernatant containing the indicated human IL2 muteins (and controls). The vertical axis is a measure of selectivity calculated as the ratio of the level of pSTAT5 activity observed on CD25 positive YT cells divided by the level of pSTAT5 activity measured on CD25 negative YT cells and each bar indicates the level of activity of the particular IL2 peptide evaluated as identified by a three letter abbreviation corresponding to the amino acids at positions 18, 22, and 126 of the hIL2 mutein numbered in accordance with wildtype hIL2 with of the with the exception of the V91K mutein which has a valine to lysine substitution at position 91.

FIG. 14 provides data in tabular form illustrating that hIL2 muteins demonstrated preferential pSTAT5 signaling activity relative to wild type hIL2 on CD25 positive YT CD25 cells relative to the CD25 negative YT cells at various dilutions.

FIG. 15 provides data relating to the cell proliferation of 3F8 cells contacted with hIL2 muteins. The figure legend indicates the different test agents and corresponding graphical symbols. Luminescence as a measure of cellular proliferation is provided on the y-axis. Protein concentration (picomolar) is provided on the x-axis.

FIG. 16 provides data relating to the interferon gamma production from 3F8 cells contacted with hIL2 muteins. Interferon gamma expression is provided on the y-axis. Protein concentration (picomolar) is provided on the x-axis. The figure legend indicates the different test agents and corresponding graphical symbols.

### DETAILED DESCRIPTION

### Abbreviations

To facilitate the understanding of the present disclosure, certain terms and phrases are defined below as well as throughout the specification. The definitions provided herein are non-limiting and should be read in view of the knowledge of one of skill in the art would know.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All patents, patent applications, and publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; pg=microgram; mg=milligram; g=gram; kg=kilogram; dl or dL=deciliter; μl or μL=microliter; ml or mL=milliliter; 1 or L=liter; pM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Unless otherwise indicated, the following terms are intended to have the meaning set forth below. Other terms are defined elsewhere throughout the specification.

Activate: As used herein the term “activate” is used in reference to a receptor or receptor complex to reflect the biological effect of the binding of an agonist ligand to the receptor. Activators are molecules that increase, activate, facilitate, enhance activation, sensitize, or up-regulate, e.g., a gene, protein, ligand, receptor, or cell. For example, the binding of an IL2 agonist to the intermediate affinity or high affinity IL2 “activates” the signaling of the receptor to produce one or more intracellular biological effects (e.g., the phosphorylation of STAT5). The evaluable parameters to parameters measure T-cell activation are well known in the art. In some embodiments, the level of activation of T-cells in response to the administration of a test agent may be determined by flow cytometric methods as described as determined by the level of STAT5 phosphorylation in accordance with methods well known in the art. STAT5 phosphorylation may be measured using flow cytometric techniques as described in in the art of using commercially available kits such as the Phospho-STAT5 (Tyr694) kit (commercially available from Perkin-Elmer/cisbio Waltham MA as Part Number 64AT5PEG) in substantial accordance with the teaching of the manufacturer.

Activity: As used herein, the term “activity” is used with respect to a molecule to describe a property of the molecule with respect to a test system (e.g., an assay) or biological or chemical property of the molecule (e.g., the degree of binding of the molecule to another molecule) or of a physical property of a material or cell (e.g., modification of cell membrane potential). Examples of such biological properties include but are not limited to catalytic activity of a biological agent, the ability to stimulate intracellular signaling, induce gene expression, induce or maintain cell proliferation, or the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” refers to an activity that promotes cell proliferation and replication, including dysregulated cell division such as that observed in neoplastic diseases, inflammatory diseases, fibrosis, dysplasia, cell transformation, metastasis, and angiogenesis.

Administer/Administration: The terms “administration” and “administer” are used interchangeably herein to refer the act of contacting a subject, including contacting a cell, tissue, organ, or biological fluid of a subject in vitro, in vivo and/or ex vivo with an agent (e.g., an αβhIL2 mutein or a pharmaceutical formulation thereof). Administration of an agent may be achieved through any of a variety of art recognized methods including but not limited to the topical, intravascular injection (including intravenous or intraarterial infusion), intradermal injection, subcutaneous injection, intramuscular injection, intraperitoneal injection, intracranial injection, intratumoral injection, intranodal injection, transdermal, transmucosal, iontophoretic delivery, intralymphatic injection (Senti and Kundig (2009) Current Opinions in Allergy and Clinical Immunology 9(6):537-543), intragastric infusion, intraprostatic injection, intravesical infusion (e.g., bladder), respiratory inhalers including nebulizers, intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell. The term “administration” includes the ex vivo contact of a cell (or population of cells) that may be isolated from a subject and contacted with an agent and the cell (or population of cells) is administered to the same subject from which the cells were obtained (autologous cell transfer) or a different subject from which the cells were obtained (allogeneic cell transfer).

Adverse Event: As used herein, the term “adverse event” refers to any undesirable experience associated with the use of a therapeutic or prophylactic agent in a subject. Adverse events do not have to be caused by the administration of the therapeutic or prophylactic agent (e.g. the IL2 mutein) but may arise from unrelated circumstances. Adverse events are typically categorized as mild, moderate, or severe. As used herein, the classification of adverse events as used herein is in accordance with the Common Terminology Criteria for Adverse Events v5.0 (CTCAE) dated published Nov. 27, 2017 published by the United States Department of Health and Human Services, the National Institutes of Health and the National Cancer Institute.

Affinity: As used herein the term “affinity” refers to the degree of specific binding of a first molecule (e.g., a ligand) to a second molecule (e.g., a receptor) and is measured by the binding kinetics expressed as Ka, a ratio of the dissociation constant between the molecule and its target (K_{off}) and the association constant between the molecule and its target (Kₒₙ).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction) induced by receptor under study, or a partial agonist. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay. The evaluation of agonist activity of the αβhIL2 muteins is made in reference to the WHO International Standard (NIBSC code: 86/500) wild type mature human IL2 evaluated at similar concentrations in a comparable assay.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway, or cell

Antibody: As used herein, the term “antibody” refers collectively to: (a) glycosylated and non-glycosylated the immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term “antibody” is not limited to any particular means of synthesis and includes naturally occurring antibodies isolatable from natural sources and as well as engineered antibodies

CD25: As used herein, the terms “CD25”, “IL2 receptor alpha”, “IL2Rα”, “IL2Ra”, the “low affinity IL2 receptor” and “p55” are used interchangeably to refer to the 55 kD polypeptide that is constituitively expressed in Treg cells and inducibly expressed on other T cells in response to activation. Human CD25 (hCD25) nucleic acid and protein sequences may be found as Genbank accession numbers NM__000417 and NP_0004Q8 respectively. The human CD25 is expressed as a 272 amino acid pre-protein comprising a 21 amino acid signal sequence which is post-translationally removed to render a 251 amino acid mature protein. Amino acids 22-240 (amino acids 1-219 of the mature protein) correspond to the extracellular domain. Amino acids 241-259 (amino acids 220-238 of the mature protein) correspond to transmembrane domain. Amino acids 260-272 (amino acids 239-251 of the mature protein) correspond to intracellular domain. The amino acid sequence of the mature form of hCD25 (without the signal sequence of the pre-protein) is:

(SEQ ID NO: 1)
ELCDDDPPEIPHATFKAMAYKEGTMLNCECKRGFRRIKSGSLYML
CTGNSSHSSWDNQCQCTSSATRNTTKQVTPQPEEQKERKTTEMQS
PMQPVDQASLPGHCREPPPWENEATERIYHFVVGQMVYYQCVQGY
RALHRGPAESVCKMTHGKTRWTQPQLICTGEMETSQFPGEEKPQA
SPEGRPESETSCLVTTTDFQIQTEMAATMETSIFTTEYQVAVAGC
VFLLISVLLLSGLTWQRRQRKSRRTI

CD122: As used herein, the terms “CD122”, “interleukin-2 receptor beta”, “IL2Rb”, “IL2β”, “IL15RP” and “p70-75” are used interchangeably to refer to the human CD122 transmembrane protein. The human CD122 (hCD122) is expressed as a 551 amino acid protein, the first 26 amino acids comprising a signal sequence which is post-translationally cleaved in the mature 525 amino acid protein. Amino acids 27-240 (amino acids 1-214 of the mature protein) correspond to the extracellular domain, amino acids 241-265 (amino acids 225-239 of the mature protein) correspond to the transmembrane domain and amino acids 266-551 (amino acids 240-525 of the mature protein) correspond to the intracellular domain. As used herein, the term CD122 includes naturally occurring variants of the CD122 protein including the S57F and D365E (as numbered in accordance with the mature hCD122 protein). hCD122 is referenced at UniProtKB database as entry P14784. Human CD122 nucleic acid and protein sequences may be found as Genbank accession numbers NM_000878 and NP_000869 respectively. The amino acid sequence of the mature hCD122 protein without the signal sequence is:

(SEQ ID NO: 2)
AVNGTSQFTCFYNSRANISCVWSQDGALQDTSCQVHAWPDRRRWN
QTCELLPVSQASWACNLILGAPDSQKLTTVDIVTLRVLCREGVRW
RVMAIQDFKPFENLRLMAPISLQVVHVETHRCNISWEISQASHYF
ERHLEFEARTLSPGHTWEEAPLLTLKQKQEWICLETLTPDTQYEF
QVRVKPLQGEFTTWSPWSQPLAFRTKPAALGKDTIPWLGHLLVGL
SGAFGFIILVYLLINCRNTGPWLKKVLKCNTPDPSKFFSQLSSEH
GGDVQKWLSSPFPSSSFSPGGLAPEISPLEVLERDKVTQLLLQQD
KVPEPASLSSNHSLTSCFTNQGYFFFHLPDALEIEACQVYFTYDP
YSEEDPDEGVAGAPTGSSPQPLQPLSGEDDAYCTFPSRDDLLLFS
PSLLGGPSPPSTAPGGSGAGEERMPPSLQERVPRDWDPQPLGPPT
PGVPDLVDFQPPPELVLREAGEEVPDAGPREGVSFPWSRPPGQGE
FRALNARLPLNTDAYLSLQELQGQDPTHLV

CD132: As used herein, the terms “CD132”, “IL2 receptor gamma”, “IL2Rg, “IL2Rγ” refers to a type 1 cytokine receptor and is shared by the receptor complexes for IL-4, IL-7, IL-9, IL-15, and IL21, hence the reference to this molecule as the “common” gamma chain. Human CD132 (hCD132) is expressed as a 369 amino acid pre-protein comprising a 22 amino acid N-terminal signal sequence. Amino acids 23-262 (amino acids 1-240 of the mature protein) correspond to the extracellular domain, amino acids 263-283 (amino acids 241-262 of the mature protein) correspond to the 21 amino acid transmembrane domain, and amino acids 284-369 (amino acids 262-347 of the mature protein) correspond to the intracellular domain. hCD132 is referenced at UniProtKB database as entry P31785. Human CD132 nucleic acid and protein sequences may be found as Genbank accession numbers: NM_000206 and NP_000197 respectively. The amino acid sequence of the mature hCD132 protein is:

(SEQ ID NO: 3)
LNTTILTPNGNEDTTADFFLTTMPTDSLSVSTLPLPEVQCFVFNV
EYMNCTWNSSSEPQPTNLTLHYWYKNSDNDKVQKCSHYLFSEEIT
SGCQLQKKEIHLYQTFVVQLQDPREPRRQATQMLKLQNLVIPWAP
ENLTLHKLSESQLELNWNNRFLNHCLEHLVQYRTDWDHSWTEQSV
DYRHKFSLPSVDGQKRYTFRVRSRFNPLCGSAQHWSEWSHPIHWG
SNTSKENPFLFALEAVVISVGSMGLIISLLCVYFWLERTMPRIPT
LKNLEDLVTEYHGNFSAWSGVSKGLAESLQPDYSERLCLVSEIPP
KGGALGEGPGASPCNQHSPYWAPPCYTLKPET

Cell Selection Process: The terms “cell selection process” and “ex vivo cell selection process” are used interchangeably to describe any of a variety of techniques for the isolation of particular subpopulations of cells from a mixed cell population based the expression or presence of one or more specific “marker” molecules in or on the cell, such as surface expressed proteins or intracellular molecules. A variety of methods for the isolation of a specific subpopulation characterized by the presence of one or more such markers may be used in the practice of the present disclosure and markers of particular cell types and subtypes may be used to isolate particular types of cells in a mixed cell population and are well known in the art. One example of a cell selection process is such method is affinity/immunoaffinity separation wherein the cells are incubated with molecule that specifically binds to such marker(s) and washing off the unbound cell types leaving the cells expressing the marker(s) of interest (positive selection) or undesired cells are retained and the cells of interest are recovered from the wash fluid (negative selection). In some embodiments, the mixed cell population is contacted with a quantity of magnetic beads conjugated to one or more binding molecules that selectively binding to the markers present on cells. Cells expressing such markers may be removed from the cell population by use of a magnet which attracts the magnetic beads to which the cells expressing the markers which are bound by the conjugated antibodies are adhered. The process is described in more detail in Molday, et al., U.S. Pat. No. 4,452,773. A variety of such antibody coated magnetic beads are commercially available under the brand names Dynabeads® or MACS® beads. In some embodiments, flow cytometry, in particular preparative scale (FACS)-sorting optionally in combination with MEMS chips (WO 2010/033140) which faciliates the isolation of T cell subpopulations at high levels of purity. Additionally, automated systems are available that provide for the isolation of specific T cell types such as the CliniMACS Prodigy system commercially available from Miltenyi Biotech.

It should be noted that current surface marker-based cell separation protocols do not generally provide for the preparation of 100% pure population of cells expressing the markers of interest but rather provides a sample that is enriched for the cells expressing the particular markers of interest. Although it is not necessary to provide a pure population of cells expressing one or more markers of interest, in some embodiments it is desirable to provide a cell population that is comprised substantially of a particular cell type. In some embodiments, the population that is comprised substantially of a particular cell type is comprised of >50%, alternatively >60%, alternatively >70%, alternatively >80%, alternatively >90% of the particular cell type. To provide additional levels of purity of the cells, it is possible subject the sample to multiple rounds of selection to approach the preparation of cells nearly entirely comprised of a cell population expressing one or more markers of interest. However, it is observed that 100% purity in the cell population is not required to prepare an efficacious cell product and the rarity of the tumor antigen experience TILS in a sample may likely be lost in such a multi-step isolation process.

Examples of surface markers that may be used to identify and an isolate particular T cell species or TIL species in a mixed population include one or more cell surface markers selected from the group consisting of CD3, CD4, CD8, CD11 a, CD11 b, CD11c, CD14, CD16, CD19, CD25, CD27, CD28, CD38 CD45RA, CD45RO, CD58, CD61, CD62L, CD66b, CD69, CD103, CD122, CD127, CD197, CD279, D62L, CD69, FoxP3, PD-1, D62L, CCR4, CCR5, CCR6(CD196), CCR7, CCR10, CXCR3, CTLA4, PD1, PDL1, TCRγδ, TCRVα24, TCRVβ11 and HLA-DR. In some embodiments, T cell subtypes are identified by the expression of one or more surface markers and populations of T cells expressing one or more surface markers are isolated by positive or negative selection techniques. Examples of subpopulations of T cells that may be isolated in accordance with such a cell selection process include CD4+ T cells, CD8+ T cells, CD25+ T cells, CD28⁺ T cells, CD62L⁺, CCR7⁺ T cells, CD27⁺ T cells, CD127⁺ T cells, CD45RA⁺ T cells, CD45RO⁺ T cells. Examples of subpopulations of T cells expressing multiple markers that may be isolated in accordance with such a cell selection process expressing multiple include but are not limited to CD25+ CD8+ T cells, CD25− CD8+ T cells, CD28⁺ T cells, CD62L⁺, CCR7⁺ T cells, CD27⁺ T cells, CD127⁺ T cells, CD45RA⁺ T cells, CD25+ CD8+ PD1+ T cells and CD62+ CD45RO⁺ T cells. In addition to cell surface markers, intracellular markers may also be used for selection of particular cell types such as Ki67, T-bet, GATA-3, PU.1, RORγt, AHR, FOXO4, and FOXP3.

Comparable: As used herein, the term “comparable” is used to describe the degree of difference in two measurements of an evaluable quantitative or qualitative parameter. For example, where a first measurement of an evaluable quantitative parameter (e.g. the level of IL2 activity as determined by an CTLL-2 proliferation or phospho-STAT5 assay) and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Derived From: As used herein in the term “derived from”, in the context of the amino acid sequence of a mutein relative to the parent version of the protein from which the mutein is derived. For example, a IL2 mutein is referred to as being “derived from” the reference wild-type IL2 polypeptide to indicate that the polypeptide or nucleic acid has a sequence that is based on that of a reference polypeptide. The term “derived from” when applied to a mutein is not meant to be limiting as to the source or method in which the mutein was derived.

Effective Concentration (EC): As used herein, the terms “effective concentration” or its abbreviation “EC” are used interchangeably to refer to the concentration of an agent (e.g., an hIL2 mutein) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect. The EC of a particular effective concentration of a test agent may be abbreviated with respect to the with respect to particular parameter and test system. For example, concentration IL2 mutein with to induce 50% of the maximal level of STAT5 phosphorylation in a CD25+ T-cell may be abbreviated as “EC₅₀^{pSTAT5-CD25+}” or similar, depending on the context. As Emax is a factor of the parameter being measured (e.g., pSTAT5 induction, proliferation), the test agent (e.g. the particular IL2 mutein such as “REH” described below) and the test system (e.g., a CD25+ human T cell, a human CD25− cell, primary human T cells), the determination of the Emax and the concentrations of the test agent sufficient to product a certain percentage of the Emax (e.g. EC₂₀, EC₅₀, etc.) may be determined empirically in the particular test system. In some instances, there are standardized accepted measures of biological activity that have been established for a molecule. For example with respect to hIL2 potency, the standard methodology for the evaluation of hIL2 potency in international units (IU) is measured in the murine cytotoxic T cell line CTLL-2 in accordance with standardized procedures as more fully described in Wadhwa, et al. (2013) “The 2nd International standard for Interleukin-2 (IL2) Report of a collaborative study” Journal of Immunological Methods 397:1-7.

EC Proliferation: The term “effective concentration sufficient to induce proliferation of CD3 activated primary human T-cells” (abbreviated herein as “EC^{PRO}”) refers to the effective concentration of an IL2 mutein sufficient induce proliferation of CD3 activated primary human T-cells as determined in accordance with the teaching of a standard protocol in the art such as using a carboxyfluorescein diacetate succinimidyl diester (CFSE) dilution assay or by thymidine incorporation. Alternatively, assess proliferation of primary human T-cells may be measured bioluminescent assay that generates a luminescent signal that is proportional to the amount of ATP present which is directly proportional to the number of cells present in culture as described in Crouch, et al. (1993) “The use of ATP bioluminescence as a measure of cell proliferation and cytotoxicity” J. Immunol. Methods 160: 81-8 or a standardized commercially available assay system such as the CellTiter-Glo® 2.0 Cell Viability Assay or CellTiter-Glo® 3D Cell Viability kits commercially available from Promega Corporation, 2800 Woods Hollow Road, Madison WI 53711 as catalog numbers G9241 and G9681 respectively in substantial accordance with the instructions provided by the manufacturer. When the abbreviation EC^{PRO} used with a subscript this is provided to indicate the concentration of the test agent sufficient to induce the indicated percentage of maximal primary human T cell proliferation in response to the test agent as measured by a given test protocol. By way of illustration, the abbreviation EC₃₀^{PRO} may be used with respect to a hIL2 mutein to indicate the concentration associated with 30% of a maximal level of proliferation of CD3 activated primary human T-cells in response with respect to such IL2 mutein as measured by the CellTiter-Glo® 2.0 Cell Viability Assay.

EC Activation: The term “effective concentration sufficient to induce activation of T-cells” (abbreviated herein as “EC^{ACT}”) refers to the effective concentration of an IL2 mutein sufficient induce activation and/or differentiation of human T-cells. When the abbreviation EC^{ACT} used with a subscript this is provided to indicate the concentration of the test agent sufficient to induce the indicated percentage of maximal STAT5 phosphorylation in a T cell in response to the application of the test agent as measured in accordance with the test protocol. By way of illustration, the abbreviation EC₃₀^{PRO} may be used with respect to a hIL2 mutein to indicate the concentration associated with 30% of a maximal level of STAT5 phosphorylation in a T cell in in response with respect to such IL2 mutein. A variety of techniques are available to one of skill in the art assess STAT5 phosphorylation such as flow cytometric methods as described Horta, et al. (2019) Oncoimmunology 8(6): e1238538, as well as through the use of commercially available kits such as the PathScan® Phospho-Stat5 (Tyr694) Sandwich ELISA Kit Commercially available from Cell Signaling Technology, Inc. (Danvers MA) as Catalog Number #7113; the STAT5A ELISA kit Commercially available from LifeSpan BioSciences (Seattle WA) as Catalog Number LS-F38421-1; the Human STAT5A ELISA Kit commercially available from Novus Biologicals (Centennial CO) as Catalog Number NVP2-80280 or the Phospho-STAT5 (Tyr694) kit (commercially available from Perkin-Elmer/cisbio Waltham MA as Part Number 64AT5PEG) in substantial accordance with the teaching of the manufacturer. When the abbreviation EC^{ACT} used with a subscript this is provided to indicate the concentration of the test agent sufficient to produce the indicated subscripted percentage of maximal STAT5 phosphorylation in a T cell in response to the application of the test agent as measured in accordance with a STAT5 protocol. By way of illustration, the abbreviation EC₃₀^{PRO} may be used with respect to a hIL2 ortholog to indicate the concentration associated with 30% of a maximal level of STAT5 phosphorylation in a T cell in in response with respect to such hIL2 ortholog as measured with the Phospho-STAT5 (Tyr694) kit.

Enriched: As used herein in the term “enriched” refers to a sample that is non-naturally manipulated so that a species (e.g. a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., as in a recombinantly modified bacterial or mammalian cell).

Extracellular Domain: As used herein the term “extracellular domain” or its abbreviation “ECD” refers to the portion of a cell surface protein (e.g. a cell surface receptor) which is outside of the plasma membrane of a cell. The term “ECD” may include the extra-cytoplasmic portion of a transmembrane protein or the extra-cytoplasmic portion of a cell surface (or membrane associated protein).

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as BLAST 2.0 algorithms, which are described in Altschul et al. (1990) J Mol. Biol. 215: 403-410 and Altschul, et al. (1977) Nucleic Acids Res. 25: 3389-3402. Software for performing BLAST analyses is publicly available through the National Center for Biotechnology Information (NCBI) web site. The algorithm involves first identifying high scoring sequence pairs (HSPs) by identifying short words of length W of the query sequence, which either match or satisfy some positive-valued threshold score “T” when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (the reward score for a pair of matching residues; always >0) and “N” (the penalty score for mismatching residues; always <0). For amino acid sequences, a scoring matrix is used to calculate the cumulative score. Extension of the word hits in each direction are halted when: (a) the cumulative alignment score falls off by the quantity X from its maximum achieved value; the cumulative score goes to zero or below, due to the accumulation of one or more negative-scoring residue alignments; or (b) the end of either sequence is reached. The BLAST algorithm parameters “W”, “T”, and “X” determine the sensitivity and speed of the alignment. The BLASTN program (for nucleotide sequences) functions similarly but uses as defaults a word size (“W”) of 28, an expectation (“E”) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, (1989) PNAS(USA) 89:10915-10919).

IL2: As used herein, the term “interleukin-2” or “IL2” refers to a naturally occurring IL2 polypeptide that possesses IL2 activity. In some embodiments, IL2 refers to mature wild type human IL2. Mature wild type human IL2 (hIL2) occurs as a 133 amino acid mature polypeptide (less the signal peptide, consisting of an additional 20 N-terminal amino acids), as described in Fujita, et al., PNAS USA, 80, 7437-7441 (1983). An amino acid sequence of naturally occurring variant of mature wild type human IL2 (hIL2) is:

(SEQ ID NO: 4)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT

As used herein, the numbering of residues of the hIL2 muteins is based on the hIL2 sequence UniProt ID P60568 excluding the signal peptide which is the same as that of SEQ ID NO:4.

IL2 Activity: The term “IL2 activity” refers to one or more the biological effects on a cell in response to contacting the cell with an effective amount of an IL2 polypeptide. As previously noted, IL2 is a pleitropic cytokine that results one or more biological effects on a variety of cell types. One example of IL2 activity may be measured in a cell proliferation assay using CTLL-2 mouse cytotoxic T cells, see Gearing, A. J. H. and C. B. Bird (1987) in Lymphokines and Interferons, A Practical Approach. Clemens, M. J. et al. (eds): IRL Press. 295. The specific activity of recombinant human IL2 (rhIL2) is approximately 2.1×10 IU/pg, which is calibrated against recombinant human IL2 WHO International Standard (NIBSC code: 86/500).

In An Amount Sufficient Amount to Effect a Change: As used herein the phrase “in an amount sufficient to effect a change” refers to the amount of a test agent sufficient to provide a detectable difference between a level of an indicator measured before (e.g., a baseline level) and after the application of the test agent to a system such as biological function evaluated in a cell based assay in response to the administration of a quantity of the test agent. “An amount sufficient to effect a change” may be sufficient to be a therapeutically effective amount but “in an amount sufficient to effect a change” may be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

Inhibitor: As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Isolated: As used herein the term “isolated” is used in reference to a polypeptide of interest that, if naturally occurring, is in an environment different from that in which it can naturally occurs. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

Metastasis: As used herein the term “metastasis” describes the spread of cancer cell from the primary tumor to surrounding tissues and to distant organs.

Modified IL2 Mutein: As used herein the term “modified IL2 muteins” is used to refer to IL2 muteins that have comprise one or more extra further modifications (i.e. modifications outside the core amino acid sequence of the IL2 mutein) such as pegylation, glycosylation (N- and O-linked), acylation, or polysialylation or by conjugation (either chemical or as fusion proteins) with other polypeptide carrier molecules including but not limited to albumin fusion polypeptides comprising serum albumin (e.g., human serum albumin (HSA) or bovine serum albumin (BSA) or and Fc-fusion proteins or with targeting moieties such as IgG comprising IL2 orthogonal polypeptide fusion proteins, targeted IL2 mutein polypeptides such as ScFv-IL2 mutein polypeptide fusion proteins and VHH-IL2 mutein polypeptide fusion proteins. Modified IL2 muteins may be prepared to order to enhance one or more properties for example, modulating immunogenicity; methods of increasing water solubility, bioavailability, serum half-life, and/or therapeutic half-life; and/or modulating biological activity. Certain modifications can also be useful to, for example, raise of antibodies for use in detection assays (e.g., epitope tags) and to provide for ease of protein purification.

Modulate: As used herein, the terms “modulate”, “modulation” and the like refer to the ability of a test agent to affect a response, either positive or negative or directly or indirectly, in a system, including a biological system or biochemical pathway.

Mutein: As used herein, the term “mutein” is used to refer to a polypeptide comprising one or more modifications to the primary structure (e.g., amino acid insertions, deletions, substitutions and modifications at one or more sites) relative to the primary structure of the parent polypeptide from which it was derived. In some instances, the parent polypeptide from which the mutein is a wild-type polypeptide. The typical terminology is to describe the mutein in reference to the parent molecule from which it was derived. Absent any particular indication that the mutein is derived from another mutein, it is assumed that the term mutein is used with respect to the wild-type form of the protein. For example, a “human IL2 mutein” would refer to a polypeptide comprising one or more modifications to the primary structure relative to the amino acid sequence of the wild-type human IL2.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. The terms “immediately N-terminal” or “immediately C-terminal” are used to refers to a position of a first amino acid residue relative to a second amino acid residue where the first and second amino acid residues are covalently bound to provide a contiguous amino acid sequence.

Neoplastic Disease: As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Operably Linked: The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

Parent Polypeptide: As used herein the terms “parent polypeptide” or “parent protein” are used interchangeably to refer to naturally occurring polypeptide that is subsequently modified to generate a variant or mutein. A parent polypeptide may be a wild-type (or native) polypeptide. Parent polypeptide may refer to the polypeptide itself or compositions that comprise the parent polypeptide (e.g., glycosylated, pegylated, fusion proteins comprising the parent polypeptide).

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. A IL2 partial agonist may have greater than 10%, alternatively greater than 20%, alternatively greater than 30%, alternatively greater than 40%, alternatively greater than 50%, alternatively greater than 60%, or alternatively greater than 70% of the activity of WHO International Standard (NIBSC code: 86/500) wild type mature human IL2 when evaluated at similar concentrations in a comparable assay.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

As used herein, the term “proliferation” refers to an increase in cell division, either symmetric or asymmetric division of cells. In particular embodiments, “proliferation” refers to the symmetric or asymmetric division of T cells. “Increased proliferation” occurs when there is an increase in the number of cells in a treated sample compared to cells in a non-treated sample.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. In some embodiments, the receptor is a cell surface receptor that comprises an extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a heteromultimeric including heterodimeric (e.g., the intermediate affinity CD122/CD132 IL2 receptor), heterotrimeric (e.g. the high affinity CD25/CD122/CD132 hIL2 receptor) or homomultimeric (e.g. homodimeric, homotrimeric, homotetrameric) complex that results in intracellular signaling.

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and may be designated as such using the abbreviation of a lower case “r” (e.g., rhIL2) to denote the method by which the protein was produced. Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors. In contrast, the terms “inhibition”, “down-regulation” and the like refer to the opposite effects.

Selective: As used herein, the term “selective” is used to refer to a property of an agent to preferentially bind to and/or activate a particular cell type based on a certain property of a population of such cells. In some embodiments, the disclosure provides IL2 muteins that are CD25 selective in that such muteins display preferential activation of cells that expressing the CD25 and/or CD25/CD122 receptors relative to the cells expressing the CD132 receptor. Selectivity is typically assessed by activity measured in an assay characteristic of the activity induced in response to ligand/receptor binding. In some embodiments, the selective IL2 mutein exhibits significantly reduced binding. In some embodiments, selectivity is measured by activation of cells expressing CD25 (e.g. YTCD25POS or YTCD25+ cells) versus the activation of that display significantly lower (preferably undetectable) levels of CD25 (e.g. YTCD25NEG or YTCD25− cells). In some embodiments, the selectivity is measured by activation of T cells expressing CD25 (e.g. Tregs) versus low levels of CD25 (e.g. non stimulated CD8+ or CD4+ T cells). In some embodiments, IL2 muteins of the present disclosure possess at least 3 fold, alternatively least 5 fold, alternatively at least 10 fold, alternatively at least 20 fold, alternatively at least 30 fold, alternatively at least 40 fold, alternatively at least 50 fold, alternatively at least 100 fold, alternatively at least 200 fold difference in EC50 on CD25+ versus CD25− cells as measured in the same assay.

Significantly Reduced Binding: As used herein, the term “exhibits significantly reduced binding” is used with respect to the affinity of the binding of a variant of a ligand (e.g. an ortholog) to a modified form of a receptor (e.g. an orthogonal CD122) relative to the binding of the variant ligand for the naturally occurring form of a receptor. In some embodiments a ligand (e.g. an ortholog) exhibits significantly reduced binding to the native form of the ligand if the orthogonal ligand binds to the native form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring ligand. Similarly and orthogonal receptor exhibits significantly reduced binding with respect to the native form of the ligand if the native form of the ligand binds to the orthogonal form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring receptor.

Specifically Binds: As used herein the term “specifically binds” refers to the degree of selectivity or affinity for which one molecule binds to another. In the context of binding pairs (e.g., a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five fold greater, alternatively at least ten fold greater, alternatively at least 20-fold greater, or alternatively at least 100-fold greater than the affinity of the first molecule for other components present in the sample. Specific binding may be assessed using techniques known in the art.

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially: As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g., blood count), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

Substantially Pure: As used herein in the term “substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

T-cell: As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

T-cell Activation Agent: As used herein, the term “T-cell activation agent” and refers to a molecule which results in the activation and proliferation of T cells regardless of the of the whether such T cells express the intermediate affinity or high affinity IL2 receptor. Examples of T-cell activation agents include cytokines, including wild-type hIL2, growth factors, antibodies to T-cell activation antigens (e.g., anti-CD3 antibodies, anti-CD137 antibodies. T cell activation agents may be used in the rapid expansion phase of the isolated TIL cell population. In some embodiments, the initial phase of the activation is conduted in the presence of an αβhIL2 mutein which provides enrichment of the cell population for antigen experienced T cells and the enriched cell population may then be generally expanded using a T cell activation agent which broadly activates T cells in the population without respect to whether they express the high affinity or intermediate affinity receptor.

Therapeutically Effective Amount: The phrase “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

Tissue Sample: As used herein, the term “tissue sample” refers to a quantity of a tissue obtained from a subject tissue from a neoplastic disease. By way of example, a tissue sample may be a quantity of a neoplasm obtained by physical disruption of the neoplasm such as by surgical (including catheter) resection and biopsy (including needle biopsy) and other similar procedures which contact the neoplasm. A “tissue sample” may also be quantity of a peripheral organs, particular organs involved in the humoral or innate immune response such as lymph nodes (particularly draining lymph nodes associated with a neoplasm), the spleen and bone marrow A tissue sample may also be a quantity of a bodily fluid such as blood (including whole blood as well as blood components such as plasma or serum), mucus secretions, saliva, cerebrospinal fluid (CSF), bronchoalveolar lavage fluid (BALF), fluids of the eye (e.g., vitreous fluid, aqueous humor), and lymph. TILs have been isolated from subjects suffering from a neoplastic disease directly via surgical resection of the neoplasm mass as well as isolated from a variety of bodily fluids (such as blood) as well as other organs which may be in communication with a tumor via the circulatory or lymphatic system such as lymph nodes (particularly draining lymph nodes) as well as from blook and blood products. PBMCs comprising TILs cells can be obtained from a unit of blood or apheresed fraction collected from a subject suffering from a neoplastic disease using any number of techniques known to the skilled person. In some embodiments, following isolation of the PBMCs from the peripheral blood as described above, the cell population may be sorted to (as described herein) to isolate a particular subpopulation of T cells such as cytotoxic CD8+ T cells and CD4+ helper T lymphocytes can be sorted into naïve, memory, and effector T cell subpopulations either before or after activation, expansion, and/or genetic modification.

Transmembrane Domain: The term “transmembrane domain” or “TM” refers to the domain of a membrane spanning polypeptide (e.g. a membrane spanning polypetide such as CD122 or CD132 or a CAR) which, when the membrane spanning polypeptide is associated with a cell membrane, is which is embedded in the cell membrane and is in peptidyl linkage with the extracellular domain (ECD) and the intracellular domain (ICD) of a membrane spanning polypeptide. A transmembrane domain may be homologous (naturally associated with) or heterologous (not naturally associated with) with either or both of the extracellular and/or intracellular domains. In some embodiments the transmembrane domain is the transmembrane domain natively associated with the ECD domain of the cognate receptor from which the orthogonal receptor is derived. In some embodiments the transmembrane domain is the transmembrane domain natively associated with the ICD domain of the cognate receptor from which the orthogonal receptor is derived. In some embodiments the transmembrane domain is the transmembrane domain natively associated with the proliferation signaling domain. In some embodiments the transmembrane domain is the transmembrane domain natively associated with a different protein. Alternatively, the transmembrane domain of the receptor may be an artificial amino acid sequence which spans the plasma membrane. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived.

Treat: The terms “treat”, “treating”, treatment” and the like refer to a course of action initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition) or ameliorates one or more symptoms associated with the presence of the disease in the subject.

Treg Cell or Regulatory T Cell. The terms “regulatory T cell” or “Treg cell” as used herein refers to a type of CD4⁺ T cell that can suppress the responses of other T cells including but not limited to effector T cells (Teff). Treg cells are characterized by expression of CD4, the a-subunit of the IL2 receptor (CD25), and the transcription factor forkhead box P3 (FOXP3) (Sakaguchi, Annu Rev Immunol 22, 531-62 (2004). By “conventional CD4⁺ T cells” is meant CD4⁺ T cells other than regulatory T cells.

Wild Type: By “wild type” or “WT” or “native” herein is meant an amino acid sequence or a nucleotide sequence that is found in nature and that has not been modified by the hand of man.

The use of TIL therapy is established for the treatment of cancers and is frequently used in the treatment of melanoma. In the basic practice of TIL therapy, a quantity of cells is isolated from a tumor sample in a subject, lymphocytes are isolated from the sample, and the isolated lymphocytes are expanded ex vivo, and the population of cells reinfused into the subject. The fundamental premise of TIL therapy is that a fraction of the lymphocytes isolated from the tumor sample (TILs) are tumor antigen specific lymphocytes which are capable of significant anti-tumor efficacy, in other words, the subject is capable of generating lymphocytes which are capable of significant anti-tumor effect. It has been observed that these desirable tumor antigen specific TILs represent a very small quantity of the total lymphocytes in the tumor sample, in some instances less than about 5% of the total quantity of T cells isolated from the tumor tissue. An additional challenge is that tumor antigen specific TILs in the tumor are “exhausted” and no longer exerting an anti-tumor effect.

Conventional TIL therapy protocols attempt to overcome these limitations by recovering a population of T cells from a tissue sample (frequently a tumor sample) comprising these tumor antigen specific TILs from a tissue sample of the subject, exposing the isolated cell population (optionally selected for the presence of cell surface markers associated with antigen experienced T cells such as CD8, CD25, CD137 and/or PD1), expanding (proliferating) and activating the isolated T cell population containing tumor antigen specific TILs ex vivo and reinfusing into the subject the expanded cell population (TIL cell product) comprising a larger number of reinvigorated tumor antigen specific TILs to the subject. It has been observed that the reinfusion of such TIL cell products prepared using established protocols results in a beneficial antitumor effect in a substantial fraction of the subjects treated.

In TIL protocols, the activation and expansion of the isolated lymphocytes cells is achieved by contacting the isolated lymphocyte cell population with a hIL2 having substantially wild-type hIL2 activity. As previously discussed, hIL2 is a pleiotropic cytokine that induces the activation and proliferation of T cells. In current clinical practice the hIL2 that is typically employed is aldesleukin, a des-Alal, C125S hIL2 mutein, which is the active pharmaceutical ingredient in Proleukin®, the US FDA-approved form of hIL2 for human use.

To further facilitate proliferation of the isolated lymphocytes, hIL2 is used in combination with an anti-CD3 and/or anti-CD28 antibody(ies) to mimic T cell activation from antigen-presenting cells. Typically, the magnetic beads to which CD3 and CD28 antibodies are conjugated are used which may be magnetically removed from the mixture. Such CD3/CD28 antibody conjugated beads are are well known in the art and are commercially available from a variety of sources (e.g., Dynabeads®, commercially available from ThermoFisher Scientific as Catalog No. 11131D; TransAct™ CD3/28 beads commercially available from Miltenyi Biotech). It is suggested that the presentation of the anti-CD3/anti-CD28 antibodies on the beads is preferred as the beads mimic the size and the three-dimensional presentation similar to antigen presenting cells.

An issue with using the conventional expansion protocol employing CD3/CD28 beads in combination with an hIL2 having substantially wild-type activity (e.g., aldesleukin) is that essentially all the lymphocytes in the isolated population are expanded indiscriminately and at approximately the same rate. As a result, the fraction of the most desired tumor antigen specific T cells in the expanded cell population remains quite small.

In order to maximize the quantity of these tumor antigen experienced T cells in the population to be reinfused into the subject to maximize the likelihood of a therapeutic effect, a very large number (e.g., a cell product comprising greater than 1×10¹⁰) of cells is typically reinfused into the patient. The administration of such a large quantity of activated lymphocytes of a mixture of cell types creates certain issues for the patient. First, there is a significant toxicity observed with the administration of a large quantity of activated lymphocytes such as autoimmune or autoimmune-like reactions (Yang, J.; Toxicities associated with adoptive T-cell transfer for Cancer (2015) Cancer J. 21:506-9; Yeh, et al. (2009) Ophthalmology 116:981-989.). Additionally, the typical practice of TIL therapy involves the immunodepletion of the subject prior to reinfusion of the activated cell product. Rohann, et al. (2018) Journal for ImmunoTherapy of Cancer 6:102). While TIL therapy in combination with lymphodepletion is correlated with an improved clinical outcome for the subject when compared to TIL therapy alone, lymphodepletion is associated with significant clinical toxicity (Yang, J., supra). Furthermore, supportive hIL2 therapy following administration of the cell product, typically with aldesleukin in clinical practice, is also associated with significant clinical toxicity.

To mitigate the toxicity arising from these well-established issues in conventional TIL therapy, a variety of ex vivo approaches have been employed to enrich the cell population for the desired antigen experienced T cells. Conventional TIL production comprises two phases: (1) an initial outgrowth phase where the isolated tissue is mechanically or enzymatically digested and are cultured in the presence of hIL2 for approximately 7-21 days (usually about 14 days), and (2) a “rapid expansion” phase where the TILs from the initial outgrowth phase are stimulated and expanded to large numbers by the contacting with a soluble anti-CD3 antibody, irradiated (autologous or allogeneic) feeder cells, and IL2 for approximately 14 days which typically results in an approximately 1000 fold expansion.

Procedures to isolate specific cell populations expressing certain cell surface molecules are well known in the art and include bead separation and fluorescent activated cell sorting (FACS) procedures. Cell sorting procedures have been employed to enrich the cell population primarily for the desired subpopulation T cells prior to expansion. For example, enrichment for CD8+ cells, (Dudley et al. (2010) Clin Cancer Research 16:6122-6131) is reported as improving clinical response. PD1 was reported to be highly expressed on the surface of tumor reactive TILs (Inozume, et al. (2010) J Immunotherapy 33:956-64) and that enrichment of the cell population for PD1+ cells was associated with an improved clinical outcome. Additionally, selection based on CD137/4-1BB expression as an activation marker for CD8+ T cells, could be used to select tumor reactive TILs from melanoma samples. Ye, et al. (2014) CD137 accurately identifies and enriches for naturally occurring tumor-reactive T cells in tumor. Clin Cancer Research 20:44-55. The administration of cell products generated using these sorting protocols are reported to provide enhanced anti-tumor response.

Notwithstanding improvements in TIL therapy arising from selection of desired cell populations, conventional TIL expansion and therapy protocols typically employ and aldesleukin which has activity similar to wild-type human IL2 (wt-hIL2). This use of wt hIL2 creates multiple issues.

First, as previously discussed, wt-hIL2 is a pluripotent cytokine broadly activates T cells in the isolated population and does not selectively activate or stimulate the proliferation of the desired the tumor antigen activated T cells. As a result, the fraction of cells that are the desired antigen experienced T cells in the cell product for reinfusion into the subject remains suboptimal.

Second, reliance on the wt-hIL2 to “support” the continued activation and proliferation of the TIL cell product following administration of the TIL cell product to the subject does not selective support for the proliferation and persistence of the antigen activated T cells and results in significant (potentially life threatening) toxicities, particularly high-dose IL2 therapy, that are well documented in the field.

Third, the failure of wt-hIL2 to selectively expand the desired antigen experienced T cells in the cell population isolated from the subject results in the generation of TIL cell product that contains a large fraction of non-tumor antigen specific T cells. In order to provide therapeutically sufficient numbers of the desired antigen activated TILs, the TIL cell product prepared using conventional methods results in the need to administer a TIL cell product containing a very large number of cells. The large quantity of cells in a conventional prepared TIL cell product often necessitates the lymphodepletion of the subject in order to enable successful the engraftment of the large quantity of cells in a conventionally prepared TIL cell product. As discussed above, the use of such lymphodepletive treatment regimens alone possess significant toxicity, often requiring treatment in the hospital environment, and leave the subject susceptible to infection from other sources.

The following is a discussion of a series of experiments conducted to demonstrate the utility of the αβhIL2 mutein in the practice of the methods of the present disclosure. Details regarding the particulars of the experiments are provided in the Examples.

The present disclosure provides αβhIL2 mutein compositions and methods of use thereof to preferentially activate tumor antigen experienced T cells in a mixed cell population. As demonstrated by the experimental data provided herein, αβhIL2 muteins selectively activate tumor specific, antigen experienced T cells in a mixed cell population.

To conduct these extensive in vitro characterization studies and in vivo studies demonstrating the utility of the αβhIL2 muteins of the present disclosure in the effective treatment of neoplastic disease in mammalian subjects, exemplary αβhIL2 muteins comprising amino acid substitutions at positions L18, Q22 and Q126 substitutions were used. As previously discussed, modification of hIL2 at positions L18, Q22 and Q126 provides a hIL2 mutein having modulated affinity to hCD132 yet typically exhibits binding to hCD25 and hCD122 comparable to wt hIL2. Two representative αβhIL2 muteins modified at positions L18, Q22 and Q126 were prepared: (1) desAla1-hREH having the amino modifications des-Ala1 L18R Q22E and Q126 (referred to as “REH” or “hREH”), and (2) desAla1-hREK having the amino modifications des-Ala1 L18R Q22E and Q126K (referred to as “REK” or “hREK”) and a surrogate murine IL2 (mIL2) mutein (mREH) comprising the amino acid substitutions L32R Q36E and Q141H (“mREH”) numbered in accordance with mature murine IL2 (UniProt P04351; SEQ ID NO:5) and corresponding to the αβhIL2 mutein comprising the amino acid substitutions L18R Q22E and Q126K of REK. Samples of the foregoing IL2 muteins polypeptides were recombinantly produced in E. coli using conventional recombinant DNA technology and isolated in substantially pure form by conventional procedures including dialysis, ion exchange chromatography and size exclusion chromatography. By deleting the alanine typically present at position 1 of the hIL2 molecule, the N-terminal methionine is more efficiently removed by the bacterial producer cell by virtue of a proline at the position next to the N terminal methionine rather than an alanine and results in the expression and recovery of a substantially more hIL2 homogenous product which provides both economic and technical advantages such as increased process efficiency, lower cost, and simplified purification and refolding to produce a substantially pure homogenous protein product which results in a more consistent reagent when additional agents such as carrier or targeting molecules are conjugated to the N-terminus of the hIL2 polypeptide. As indicated in earlier reports and confirmed by the present studies, elimination of the alanine at position 1 does not substantially modify the biological activity of the resultant hIL2 polypeptide. The αβhIL2 mutein test agents were prepared in substantial accordance with the teaching of the Examples

To demonstrate the effectiveness of αβIL2 muteins to preferentially expand tumor antigen experienced T cells in an isolated mixed cell population, a series of experiments were conducted in a mouse model demonstrating that a population of immune cells extracted from a tumor tissue when cultured ex vivo in the presence of an αβIL2 mutein preferentially expands tumor antigen experienced lymphocytes in a mixed cell population. The parameters involved in the mouse model are provided in the Examples.

To model the activity of the human αβIL2 mutein in a mouse environment, a murine αβIL2 mutein was prepared and evaluated to demonstrate comparable activity. As data presented separately herein, the human αβIL2 mutein comprising the amino acid substitutions L18R Q22E and Q126K (“REK”) has significant anti-tumor efficacy in human tumor models.

To demonstrate that the murine REH αβIL2 mutein is an appropriate a surrogate to the human REK for use in mouse studies, the ability of REH and REK to provide signaling via the IL2 receptor as evaluated by phosphorylation of STAT5 was evaluated in YT CD25 (CD25 positive) and YT (CD25 negative) cells. Briefly, 293T cells were transfected with IL2 mutein constructs and after 2-3 days, supernatants containing the soluble REK and REH IL2 muteins were removed. The supernatants were added to YT and YT CD25 cells following a 20 minute stimulation. YT cells are a NK lymphoma cell line, which does not endogenously express detectable levels of CD25. IL2 responses of YT cells and a derivative YT cell, exogenously expressing CD25 (“YT CD25) were compared. Untransfected cells, cells transfected with an empty expression cassette and wild-type human IL2 were included in this study as controls. The pSTAT5 levels were measured by flow cytometry. IL2 concentration in the supernatants measured by MSD assay. The mean fluorescent intensity data generated from this experiment is provided in Tables 2 and 3 below.

TABLE 2
pSTAT5 MFI in treated YT CD25+ Cells

| Supernatant | | | | | wt | | | | | |
|---|---|---|---|---|---|---|---|---|---|---|
| Dilution | Untransfected | | Empty | | hIL2 | | REH | | REK | |
| 1:2 | 991 | 1058 | 1021 | 1015 | 21882 | 20337 | 22248 | 23922 | 21176 | 22404 |
| 1:4 | 1094 | 1084 | 1180 | 1080 | 20568 | 22024 | 20858 | 22407 | 21255 | 21241 |
| 1:8 | 1069 | 1150 | 1019 | 1084 | 21583 | 21557 | 21464 | 21675 | 20648 | 21053 |
| 1:16 | 1133 | 1048 | 1158 | 1067 | 19846 | 22331 | 20627 | 20841 | 20951 | 20588 |
| 1:32 | 1175 | 1081 | 1122 | 1044 | 19023 | 20522 | 21164 | 21890 | 19189 | 19600 |
| 1:64 | 1065 | 1015 | 1057 | 1120 | 20967 | 20823 | 20651 | 21139 | 20930 | 19283 |
| 1:128 | 1068 | 1090 | 1099 | 1044 | 18118 | 21921 | 23158 | 20999 | 18968 | 19482 |

TABLE 3
P-STAT5 MFI in treated YT (CD25 NEGATIVE) Cells

| Supernatant | | | | | wt | | | | | |
|---|---|---|---|---|---|---|---|---|---|---|
| Dilution | Untransfected | | Empty | | hIL2 | | REH | | REK | |
| 1:2 | 959 | 988 | 930 | 941 | 25483 | 26572 | 24955 | 26085 | 20485 | 20835 |
| 1:4 | 931 | 1015 | 924 | 856 | 23197 | 23079 | 21713 | 23060 | 12843 | 13798 |
| 1:8 | 868 | 952 | 920 | 843 | 21160 | 22836 | 19475 | 18404 | 8374 | 7907 |
| 1:16 | 961 | 926 | 852 | 1093 | 22670 | 21882 | 17282 | 15044 | 4363 | 4280 |
| 1:32 | 983 | 912 | 886 | 917 | 21323 | 21734 | 12086 | 11940 | 2564 | 2467 |
| 1:64 | 964 | 884 | 920 | 925 | 19220 | 19045 | 7456 | 6895 | 1834 | 1579 |
| 1:128 | 976 | 999 | 902 | 982 | 17318 | 18179 | 4766 | 4711 | 1551 | 1386 |

As the foregoing data demonstrates, REH and REK, provide selective activation of CD25 positive T cells relative to CD25 negative T cells as demonstrated by enhanced pSTAT5 production in an immune cell expressing the high affinity trimeric CD25/CD122/CD132 hIL2 receptor (YT CD25 cells) relative to pSTAT5 in an immune cell expressing the intermediate affinity dimeric CD122/CD132 hIL2 receptor, YT cells.

To further validate mREH as a valid surrogate of hREK, a study was performed to evaluate the relative potencies of human wild type IL-2 (huIL-2), REH and REK by comparing the EC₅₀ of each molecule in inducing phospho-STAT5 (pSTAT5) in primary human CD8+ T cells, activated by anti-CD3/anti-CD28 stimulation, and in primary human NK cells. Both cell types were isolated from fresh donor peripheral blood monocytic cells (PBMCs). As the REH is a murine IL2 mutein, it was tested on equivalent cell populations freshly isolated from mouse spleen. The results of this study are provided in Table 4 below:

TABLE 4
Potency of Human and Murine IL2 muteins in pSTAT5 Assay

| | | STK- | |
|---|---|---|---|
| Test Condition | hIL2 | 012 | mREH |
| CD25+ CD8+/NK selectivity (p- | 5.6 | >10³ | >10⁶ |
| STAT5) | | | |
| CD25+ CD8+/CD25− CD8 selectivity (p- | 1.75 | >10³ | >10⁶ |
| STAT5) | | | |

The data provided in Table 4 demonstrates that REH represents a valid surrogate for REK for use in in vivo efficacy models as REK possesses a similar target specificity on mouse cells as REK exhibits on human cells.

Having established the REH mIL2 mutein as an appropriate murine surrogate for the REK αβhIL2 mutein, a study was conducted in a murine environment to evaluate the effectiveness of REH in selectively expanding tumor antigen specific T cells from a murine MC38 tumor. The MC38 tumor cell line is derived murine colon adenocarcinoma cells and forms neoplastic lesions when implanted in mice. The detailed protocol for this experiment is provided in the attached Examples. Briefly, MC38 tumor cells were implanted subcutaneously into a series of female C57/Bl6 mice. On day 14 following implantation of the tumor cells, the mice were sacrificed, and the tumors harvested. The isolated tumors were enzymatically digested and CD4+ and CD8+ T cells were isolated resulting in approximately 4×10⁷ CD4+ and CD8+ T cells from 55 tumors. The CD4+ and CD8+ T cells obtained were cultured in the presence of wild-type murine IL2 (wt-mIL2) or REH (a murine surrogate for the REK human ap biased hIL2 mutein). Wild type IL2 was included as a control. Approximately, 5-7 days later, cells were harvested and plated together with tumor target cells. MC38 tumors cells were used as the target cell line expressing cognate tumor antigens, while B16 were used as a strain matched C57BL/6 negative control. Cells were incubated with target cells overnight; 16 hours later, protein transport inhibitor monensin was added for 4-5 hours. After this incubation, cells were stained for flow cytometry analyses. To contrast the expansion of activated cells using the αβ biased mIL2 mutein REH expansion was different than wt-mIL2, live, CD8+ T cells were isolated by FACS and then IFN-gamma expression analyzed.

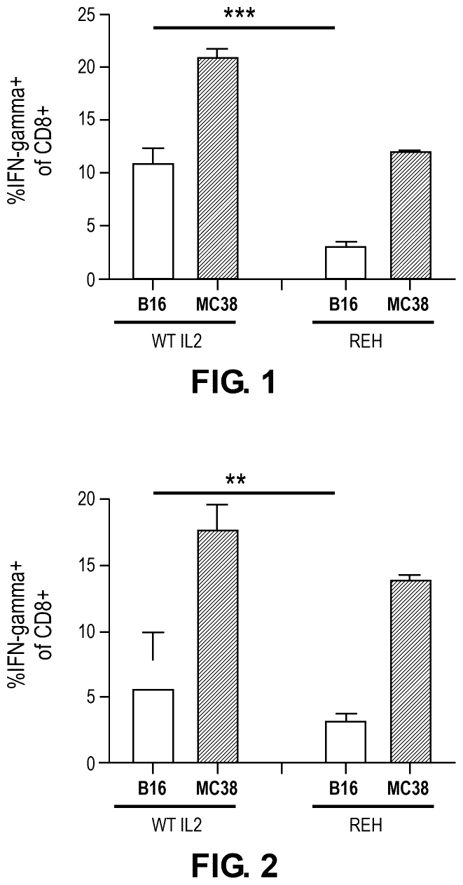

The results of these experiments are provided in FIGS. 1 and 2 of the attached drawings. As illustrated, wt-mIL2 results in non-selective expansion of T cells increasing proliferation of T cells that respond to both MC38 cells and the strain matched B16 cells. In contrast, culture of the antigen activated T cells in the REH αβIL2 mutein expanded T cells responding to the MC38 cells but significantly less to the strain matched B16 cells significantly less. This data demonstrates that TILs expanded in the presence of an αβIL2 mutein result in the selective expansion of antigen activated TILs that specifically bind to the antigens provided on the tumor cells in the subject from which the TIL cells were isolated. Consequently, the use of αβIL2 muteins in the ex vivo expansion of TILs in the preparation of a TIL cell product results in a TIL cell product that is specifically enriched for antigen activated TIL that are specific for the tumor. As this nature of the immune response in humans is to generate a polyclonal immune response the isolated TILs provide multiple T cell clones reactive with the tumor antigen and the ability of αβIL2 muteins to selectively activate antigen activated TILs provides a TIL cell product comprising a plurality of tumor antigen specific T cell clones. The methods and compositions of the present invention enable the provision of an enhanced polyclonal immune response in the treatment of a neoplastic disease by facilitating the generation of a population of enriched for plurality of tumor antigen specific T cell clones.

The foregoing discussion demonstrates the ability of the compositions and methods of the present disclosure are useful ex vivo to prepare a TIL cell product that is enriched for tumor antigen specific activated T cells. The compositions and methods of the present disclosure further provide a method of activating antigen activated TILs in vivo, maintaining and further expanding the antigen activated TILs.

In a preferred embodiment, the TIL cell product used in combination with the αβhIL2 muteins is prepared in accordance with the foregoing method providing a TIL cell product that is substantially enriched for tumor antigen specific activated TILs, however an αβIL2 mutein may be administered to a subject in combination with TIL cell products that are prepared using conventional TIL preparation protocols such as the selected TIL method or young TIL methods described herein.

As discussed, the administration of wt-hIL2 to a subject in combination with TIL therapy does not provide for selective support for the proliferation and persistence of the antigen activated T cells and results in significant (potentially life threatening) toxicities, particularly high-dose IL2 therapy. A series of experiments was conducted that demonstrate that: (1) the αβhIL2 muteins of the present disclosure selectively activate antigen activated CD8+ T cells in vivo and (2) αβhIL2 muteins do not result in the systemic toxicities associated with the administration of wt-hIL2, the standard of care in the field.

As described herein a series of αβhIL2 muteins of were prepared. A representative αβhIL2 mutein comprising a deletion of the N-terminal alanine residue and the amino acid substitutions L18R, Q22E and Q126K (“hIL2-REK”) and its murine surrogate mREH (as described above) were selected for evaluation in primate and murine systems.

The ability of the αβhIL2 mutein to activate and proliferate TCR activated T cells was evaluated in vitro and in vivo. The in vivo half-life of wild-type IL2 molecules (including most IL2 muteins) is short, typically on the order of minutes, in a mammalian subject. To improve their pharmacokinetic properties, IL2 molecules are frequently modified to provide for extended half-life in vivo. Various methodologies for extending the in vivo half-life of IL2 molecules are applicable to the αβ-IL-2 muteins and are discussed in more detail below. A series of in vivo experiments was conducted to demonstrate the selectivity of the αβ-IL-2 mutein hREK and its murine surrogate mREH wherein the αβ-IL-2 human and murine muteins were modified by the N-terminal covalent attachment of a 40 kDa branched (2×20 kDa) polyethylene glycol (“PEG”) moiety to the IL2 mutein using conventional aldehyde chemistry.

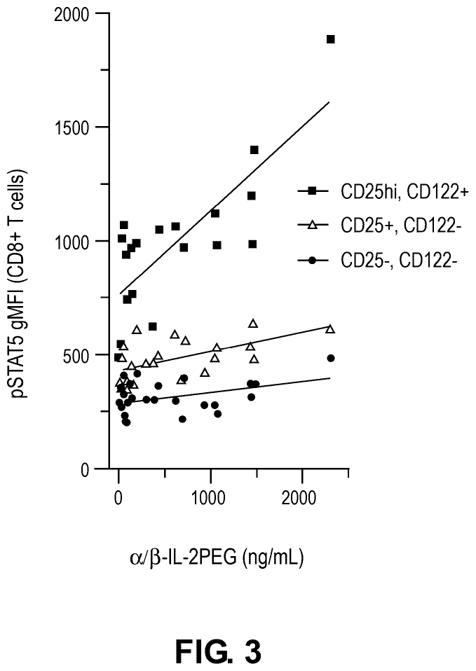

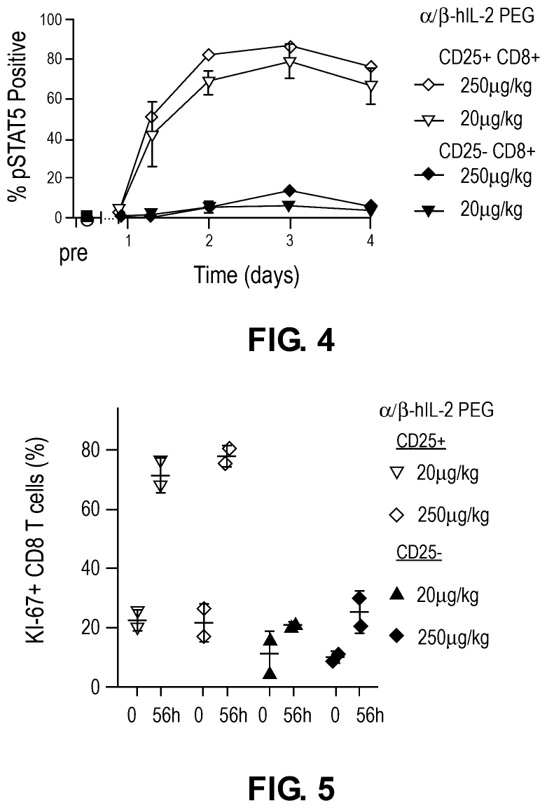

The αβ-IL-2-PEG was evaluated in a non-human primate. As a comparator and to illustrate that retention is of CD25 binding in the hIL2 mutein is a factor in the expansion of antigen activated cells, the primate was also dosed with a similarly PEGylated version of the neo-2/15 non-a-IL2 mutein described in Silva, et al. (2019) Nature 565:186-19 (“non-α-hIL2-PEG”). As illustrated by the data presented in FIG. 3, αβ-IL-2-PEG induced STAT5 phosphorylation preferentially in CD25^{h}i CD122+ CD8+ T cells and substantially not in CD25+ CD122- or CD25− CD8+ T cells. The data presented in FIG. 4 of the attached drawings demonstrates that the ability of the PEGylated αβhIL2 mutein to selectively activate CD25+ CD8+ T cells was maintained over a wide dose range. As illustrated, the PEGylated αβhIL2 analog provided sustained high levels of STAT5 phosphorylation of CD25+ CD8+ T cells at all doses evaluated. In contrast, the PEGylated αβhIL2 mutein resulted in significantly lower levels of STAT5 phosphorylation in the CD25^{neg} CD8+ T cells at both doses. These data demonstrate that the PEGylated αβhIL2 muteins provide sustained activation of CD8+ T cells and that such activity is regulated by the expression of CD25 on such CD8+ T cells.

The data presented in FIG. 5 of the attached drawings demonstrates that the αβhIL2 mutein αβ-IL-2-PEG induced proliferation of CD25+ CD8+ T cells directly within the first days after injection. As illustrated, at the 56-hour time point, the at both the 250 μg/kg and 20 μg/kg dose, the PEGylated αβhIL2 mutein resulted in a significant increase the percentage of KI-67+ CD8+ T cells in the sample. In contrast, at the 56-hour time point, the at both the 250 μg/kg and 20 μg/kg dose, the PEGylated αβhIL2 mutein results in only a very minor increase the percentage of K167+ CD8+ T cells in the sample. These data demonstrate that the PEGylated αβhIL2 muteins induce proliferation of CD8+ T cells and that such proliferation is regulated by the expression of CD25 on such CD8+ T cells.

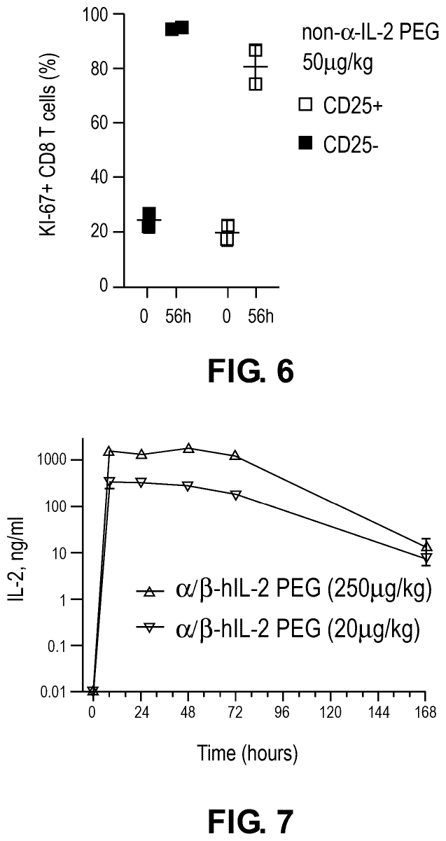

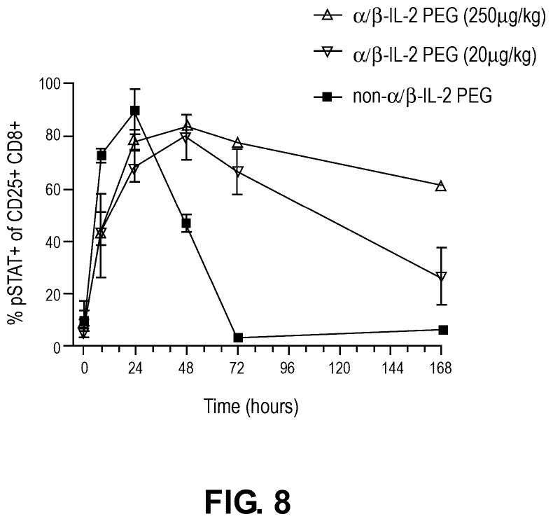

In contrast, as illustrated in FIG. 6, the non-α-IL-2-PEG equally induced the proliferation of both CD25⁺CD8⁺ T cells and CD25⁻CD8⁺ T cells demonstrating the inability of such agents to selectively stimulate the proliferation of the CD25⁺CD8⁺ T cells. These data illustrate that the PEGylated non-a hIL2 mutein induces the proliferation of both CD25^{neg} and CD25^{pos} CD8+ T cells at a dose of 50 μg/kg. These data demonstrate that the PEGylated a non-α hIL2 mutein results in activation of CD8+ T cells regardless of the CD25 status and does not provide selective activation of CD25+ CD8+ T cells as observed with the PEGylated αβhIL2 as illustrated in FIG. 5.

To illustrate the prolonged in vivo half-life of the PEGylated αβhIL2 mutein, serum samples were obtained from non-human primate model discussed above and the levels of IL-2 in the samples determined. The results of the study are presented in FIG. 7 of the attached drawings. The data presented in FIG. 7 illustrates that the PEGylated αβhIL2 mutein the at both the 250 μg/kg and 20 μg/kg dose provides sustained serum levels greater than about 10 ng/ml over a period of 168 hours in response to a single subcutaneous administration of the PEGylated αβhIL2 mutein.

To evaluate the duration of action PEGylated αβhIL2 mutein, the samples obtained above from primates treated with PEGylated non-α-hIL2 mutein and the PEGylated αβhIL2 evaluated for the percentage of CD25+ CD8+ cells that were activated (as indicated by STAT5 activity, y-axis) over time (x-axis). The data show that the percentage of STAT5+ CD8+ CD25+ T cells following subcutaneous administration of a dose of either 250 μg/kg or 20 μg/kg of the PEGylated αβhIL2 mutein provides sustained activation of CD25+ CD8+ over the time course of the study (168 hours or 7 days) at significant levels. In contrast, the percentage of STAT5+ CD8+ CD25+ T cells diminished rapidly following the administration of the PEGylated non-α-hIL2 mutein. Collectively this data demonstrates that not only does the PEGylated αβhIL2 mutein possesses an extended lifetime in vivo but it is also maintained at a level where the agent provides a significant activating effect on CD8+ CD25+ T cells over this period.

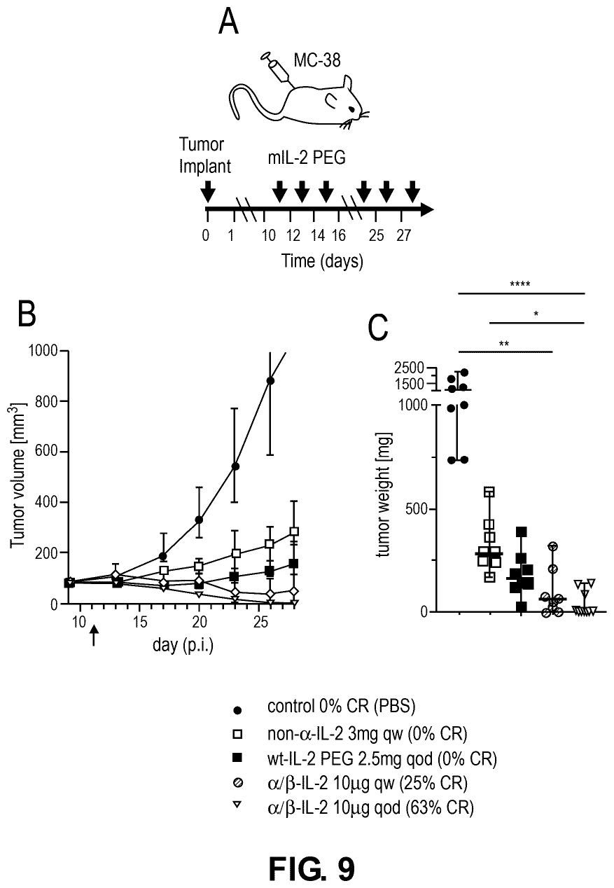

To illustrate that αβhIL2 muteins possess anti-tumor activity in vivo and that the administration of this activity correlates with an observed increase in CD8+ CD25+ TILs in response to the αβhIL2 mutein, a series of studies was conducted in the mouse, using the MC38 mouse tumor and using murine IL2 muteins. Initially, the potency of the different IL2 muteins was evaluated in the mouse MC38 tumor model in substantial accordance with the MC38 model described above and in the Examples. The study design and dosing schedule are described in Panel A of FIG. 9 of the attached drawings. Mice were treated with 10 g of the murine PEGylated αβ(REH) mIL2 at different dosing regiments; a PEGylated wild-type murine IL2 at a dose of 2.5 mg and a PEGylated murine version of neo2/15 molecule was dosed at 3 mg. As Illustrated in Panel B of FIG. 9 mice treated with the highest non-lethal dose regimen of wt-mIL2-PEG reduced the growth of syngeneic MC-38 mouse colon carcinomas but did not result in any complete responses (CRs). As illustrated in FIG. 9, Panels B and C, the non-α-IL2-PEG was less efficacious than mIL-2-PEG and did not induce complete response in the mice. In contrast, αβ-IL2-PEG induced complete response in more than 50% of the treated mice. Consequently, in addition, to providing specific support of the TIL cell product and enhancing the persistence of the antigen activated T cells, the administration of αβIL2 muteins are useful as a monotherapy, or in combination with other supplemental agents in the treatment of neoplastic disease.

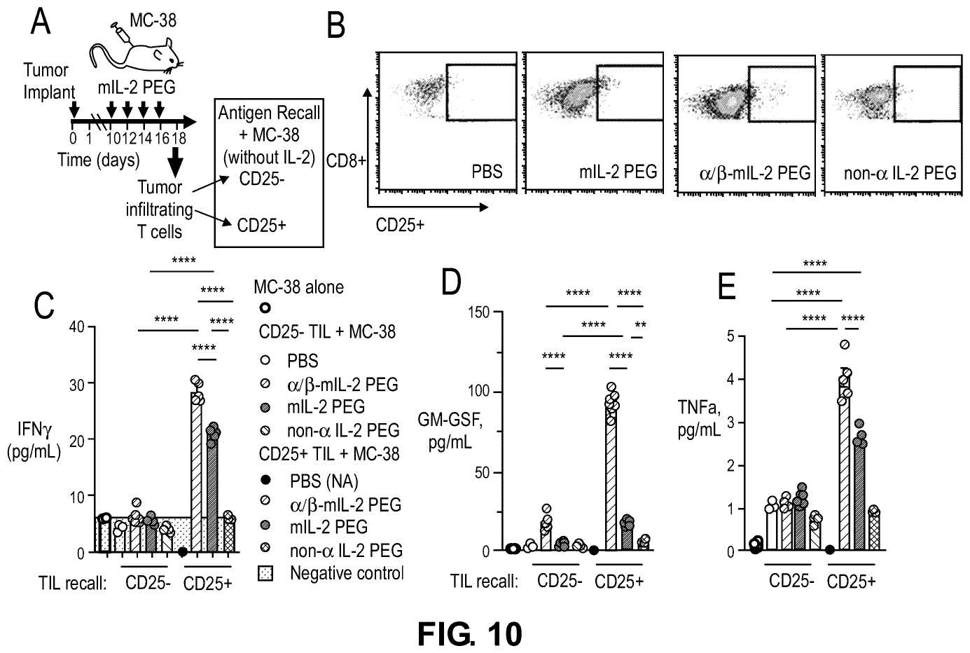

T cell responses to tumor derived neo-antigens are thought to facilitate anti-tumor responses in patients. Rizvi, et al. (2015) Science 348:124-128. The αβ-IL2-PEG muteins are designed to preferentially target antigen activated CD25+ T cells. A study in mice was conducted to demonstrate that αβ-IL2 muteins selectively activates tumor antigen specific CD8+ tumor infiltrating T cells (TILs). The study design and results are presented on FIG. 10 of the attached drawings. Briefly mice were injected with MC38 tumor cells and treated with various test agents (PBS, αβ-mIL2-PEG mutein, pegylated wild type murine IL2 (mIL2 PEG) and a PEGylated non-α-IL2 on the schedule shown in FIG. 10, Panel A. On day 18 of the study, the tumors from each treatment group were harvested, the lymphocytes were isolated from tumors (TILs) corresponding to each treatment group and the isolated cell populations were further sorted for CD25 expression into two subpopulations of CD8+ CD25+ and CD8+ CD25− T cells. Each subpopulation was exposed to ex vivo to MC38 tumor cells and levels of IFNg, GM-CSF and TNFα in each population in response to re-exposure to the MC38 tumor cells. The results of this study are presented in Panels C, D and E of FIG. 10. As illustrated, T cells isolated from the tumors of the mice exposed to the CD25− CD25+ TILs did not secrete IFNg in response to tumor exposure, while CD25+TILs from αβ-IL2-PEG treated mice secreted IFNg, GM-CSF and TNFα at high levels. In these studies, mIL2-PEG showed a reduced cytokine secretion compared to αβ-IL2-PEG, while non-α-IL2-PEG failed to support antigen reactive T cells. It should be noted that the tumor cell specific activity of TILs associated with the various treatment agents correlated with their respective efficacies observed in the MC-38 tumor efficacy model data presented in FIG. 9.

Apart from the significant toxicities of high dose (HD-)hIL2 therapy used in the context of TIL therapy discussed above, wt-hIL2 is non-selective and consequently reliance on the wt-hIL2 to “support” the continued activation and proliferation of the TIL cell product following administration of the TIL cell product to the subject does not provide for enhanced persistence of the tumor antigen activated cells. As described in more detail below, the administration of an αβIL2 muteins in combination with the reinfusion of a TIL cell product provides specific support for the antigen activated T cells and further mitigates or avoids the toxicities associated with the administration of wt-hIL2, in particular, HD-hIL2 therapy.

Wt-hIL2 activates the high affinity trimeric IL2 receptor (IL2Rαβ/γ) present on antigen activated T cells and regulatory T cells (Tregs) or an intermediate affinity as well as the intermediate affinity dimeric receptor (IL2Rβ/γ) expressed on naïve and resting T cells and NK cells. The αβIL2 muteins preferentially activate cells expressing the high affinity trimeric receptor (such as tumor specific CD25+ CD8+ T cells) relative to cells expressing the intermediate affinity receptor (such as NK cells).

Acute toxicity in hIL2 treated patients includes vascular leak syndrome (VLS), resulting in edema in peripheral tissues and the lung, limiting blood oxygenation. Patients on high dose IL2 therapy (HD-IL2) may start to experience significant toxicity after two days and may require supportive care. Dutcher, et al. (2014) Journal for immunotherapy of cancer 2:26. Two prevalent opposing hypotheses are that VLS is either be mediated by CD25+ endothelial cells (Krieg, et al. (2010) PNAS(USA) 107:11906-11911) or the extravasation of CD25− NK cells and granulocytes (Peace and Cheever (1989) J Exp Med 169:161-173). To evaluate these parameters, non-human primates were exposed for 56 hours to three different IL2 agents: (a) hIL2 having wild-type hIL2 activity which is conventionally used in the clinic (Proleukin®, Prometheus Laboratories) referred to as “wild-type hIL2”; (b) a PEGylated representative ap-hIL2 mutein comprising the amino acid substitutions L18R, Q22E and Q126K (“ap-hIL2-PEG”), and (c) a PEGylated version of the neo-2/15 non-α-IL2 mutein described in Silva, et al. (2019) Nature 565:186-19 (“non-α-hIL2-PEG”). The PEGylated versions of the ap-hIL2 and a non-α-IL2 were modified by covalent N-terminal attachment of a 40 kDa, 2-arm branched PEG (NOF #SunBright GL2-400AL3) using conventional aldehyde chemistry. The non-α-hIL2-PEG was administered intravenously at a dose of 50 micrograms/kilogram in three doses on days 1, 8 and 15 of the study. The ap-hIL2-PEG was administered subcutaneously at a dose of 250 micrograms/kilogram in three doses on days 1, 8 and 15 of the study. Eight doses of Proleukin® were administered intravenously at a dose of 37 micrograms/kilogram three times per day period of 8 days. Acute toxicity was evaluated at 56 hours post the conclusion of treatment. Toxicity was evaluated by immunohistochemistry for immune cell composition in the lung. Chronic toxicity after three weekly doses of each PEGylated agent while the wt hIL2 was dosed three times per day for 8 doses to mimic the conventional clinical HD-hIL2 therapy.

Animals treated with wt-IL2 or PEGylated non-α-IL2 showed significant infiltration of CD11b+ neutrophils in the lungs which corresponded histologically to pulmonary edema. In particular, the non-α-IL2-PEG induced strong sub-endothelial infiltrates of CD11b+ granulocytes. Treatment with wt-IL2 or non-α-IL2-PEG resulted increased the number of CD3+ T cells and NK cells (Granzyme B+ CD3− cells) as well as IFNγ expression in NK cells and their proliferation in the lung. In contrast, αβ-IL2-PEG did not increase CD11b+ cells nor was there a significant change in cell infiltrates in response to treatment with the αβ-hIL2 PEG.

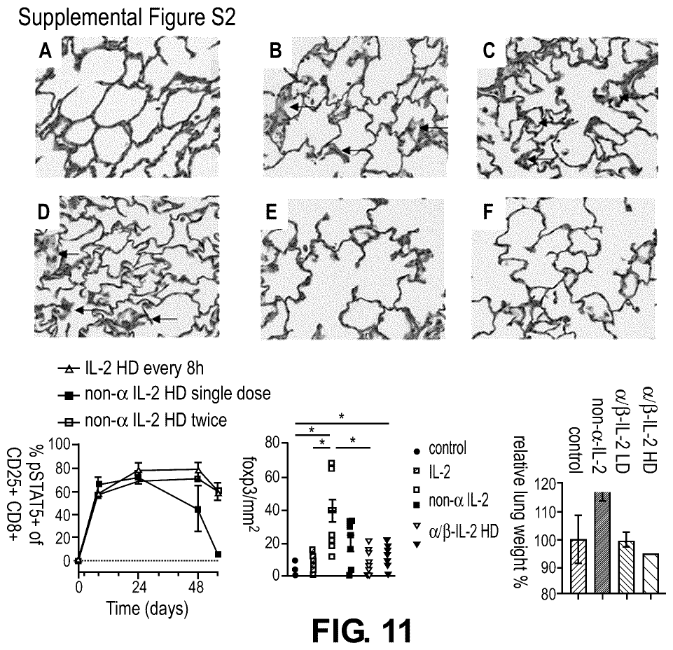

Additional results of this study are presented in FIG. 11 of the attached drawings. FIG. 11, Panels A-F show the lung histology in response to the various PEGylated hIL2 muteins. On day 3 of IL-2 treatment (FIG. 2), showing alveolar thickening (arrows) and cell infiltration in response to aldesleukin (Panel B) and non-α-IL-2-PEG (Panel C, 1 dose; Panel D, 2 doses) but not in the control (Panel A) or with αβ-IL-2-PEG (Panel E and Panel F). Continuous phospho-STAT5 induction by HD-IL-2 (every 8 h) or 2 subsequent doses of non-α-IL-2 PEG, but phospho-STAT5 duration limited to 48h after one dose of non-α-IL-2 PEG (Panel G) and that non-α-IL-2-PEG Treg infiltration in the lungs on day 3 (Panel H). Weekly, chronic dosing with non-α-IL-2-PEG in Part B of the study induced similar CD11b+ infiltrates (not shown) and increased the relative weight of the lungs compared to controls or αβ-IL-2-PEG FIG. 11, Panel I.

The foregoing data generated in a non-human primate demonstrates that the αβIL2 muteins of the present disclosure provide significantly reduced toxicity in comparison to wild-type hIL2 therapy or non-α-hIL2 muteins, that prolonged exposure to αβhIL2 muteins of the present disclosure does not result in the systemic toxicities associated with wt-hIL2 therapy or non-α-hIL2 muteins.

The αβ-IL2s muteins which have substantially reduced binding to the dimeric intermediate affinity CC122/CD132 (IL2Rβ/γ) IL2 receptor have an improved safety profile compared to wt hIL2 or IL2 muteins which have been modified provide reduced binding to the CD25 component of the high affinity trimeric IL2 receptor (referred to as “non-α-IL2 muteins”) because they have have substantially reduced binding to the dimeric IL2Rβ/γ receptor complex and consequence do not substantially activate or proliferate cells expressing the dimeric IL2Rβ/γ receptor thereby avoiding direct NK cell activation and vascular leak toxicity. αβ-IL-2s also have an improved safety profile compared to wt and non-α-IL-2 because they do not activate cells expressing the dimeric IL-2Rβ/γ, thereby avoiding direct NK cell activation and vascular leak toxicity.

The present disclosure provides the use of αβhIL2 muteins in the practice of TIL therapy in both, or either, of the ex vivo cell expansion phase and the support of the TIL cell product. The TIL cell product enriched for tumor antigen specific T cell clones generated using the compositions and methods of the present disclosure are useful in the ex vivo preparation and in vivo support of a polyclonal antitumor immune response in a subject. The ex vivo preparation TIL cell product using αβhIL2 muteins provides a method of preparing a TIL cell product substantially enriched for tumor antigen specific activated T cells. The αβhIL2 muteins of the present disclosure provide selective in vivo support of the tumor antigen specific activated T cell clones facilitating a polyclonal antitumor immune response useful in the treatment of neoplastic diseases. The use of αβhIL2 muteins in the ex vivo preparation of TIL cell products provides a cell product significantly enhanced for antigen activated T cells either obviating the need for lymphodepletion of the subject prior to administration of the TIL cell product or enabling the use of less aggressive forms of lymphodepletion of the subject prior to administration of the TIL cell product.

Although the ex vivo use of the αβhIL2 muteins produces a TIL cell product that is substantially enriched for activated tumor antigen specific T cells, the αβhIL2 muteins may also be used in combination TIL therapy where the TIL cell product was prepared using conventional TIL preparation protocols. In one embodiment, the present disclosure provides a method of treating a subject by the administration to the subject of a therapeutically effective amount of an αβhIL2 muteins in combination with the administration of a TIL cell product, wherein the TILs in the TIL cell product were expanded using conventional methodologies employing wt-hIL2 or using an αβhIL2 mutein.

The compositions and methods of the present disclosure relates to IL2 muteins. Unless otherwise specified, the following terminology and conventions are used in relation to such IL2 muteins.

In some embodiments, the αβhIL2 mutein useful in the practice of the present disclosure is an IL2 mutein having 85% or greater sequence identity, alternatively 90% or greater sequence identity, alternatively 91% or greater sequence identity, alternatively 92% or greater sequence identity, alternatively 93% or greater sequence identity, alternatively 94% or greater sequence identity, alternatively 95% or greater sequence identity, alternatively 96% or greater sequence identity, alternatively 97% or greater sequence identity, alternatively 98% or greater sequence identity, 90% or greater sequence identity to wt-hIL2 (SEQ ID NO:4), the αβhIL2 mutein comprising one or more amino acid substitutions at positions 18, 22 and 126 numbered in accordance with wt-hIL2 (SEQ ID NO:4).

In some embodiments, the αβhIL2 mutein useful in the practice of the present disclosure comprises a deletion of 1, 2, 3, 4, 5, 6, 7, 8, 9, or more N-terminal amino acid residues. In some embodiments, the αβhIL2 mutein useful in the practice of the present disclosure comprises a deletion of 1, 2, 3, 4, 5, 6, 7, 8, or 9 N-terminal amino acid residues. In some embodiments, the αβhIL2 mutein useful in the practice of the present disclosure comprises a deletion of 1, 2, 3, 4, or 5 N-terminal amino acid residues. In some embodiments, the αβhIL2 mutein useful in the practice of the present disclosure comprises a deletion of 1, 2, or 3 N-terminal amino acid residues. In some embodiments, the αβhIL2 mutein useful in the practice of the present disclosure comprises a deletion of the N-terminal alanine amino acid residue (abbreviated des-Alal).

As used herein the terms “alpha/beta biased IL2 mutein” and “a/P biased IL2 mutein” and “αβIL2 mutein” are used interchangeably herein to refer to an IL2 polypeptide comprising one or more structural modifications (e.g., a primary structural modification comprising one or more amino acid substitutions, modifications, or deletions) that possess significantly reduced binding affinity for the CD132 subunit of the IL2 receptor but retains substantially of wild-type binding affinity the CD25 and CD122 subunits of the IL2 receptor. As used herein the terms “alpha/beta biased hIL2 mutein” and “α/β biased hIL2 mutein” and “αβhIL2 mutein” are used interchangeably herein to refer to an hIL2 polypeptide comprising one or more structural modifications (e.g., a primary structural modification comprising one or more amino acid substitutions, modifications, or deletions) that possess significantly reduced binding affinity for the hCD132 subunit of the hIL2 receptor but retains binding affinity comparable to of wild-type hIL2 for the hCD25 and hCD122 subunits of the hIL2 receptor.

Binding affinity for of the hIL2 muteins may be assessed with respect to one or more subunits IL2 receptor (e.g., CD25, CD122 and/or C132) may be determined by techniques known in the art. As used herein, when reference is made herein to the binding affinity of an IL2 mutein for a IL2 receptor subunit, the binding affinity is determined by surface plasmon resonance (“SPR”). In evaluating binding affinity of an IL2 mutein for a IL2 receptor subunit, either member of the binding pair may be immobilized, and the other element of the binding pair be provided in the mobile phase. In some embodiments, the “chip” on which the protein of interest is to be immobilized is conjugated with a substance requiring the derivatization of the protein to be immobilized as anti-His tag antibodies, protein A or biotin. Consequently, in order to evaluate binding, it is frequently necessary to modify the protein to provide for binding to the substance conjugated to the surface of the chip. For example, the IL-2 mutein may be modified by incorporation of a poly-histidine sequence for retention on a chip conjugated with an anti-his tag antibody (e.g. anti-histidine CM5 chips commercially available from Cytiva, Marlborough MA). Alternatively, the IL2 receptor component may be immobilized on the chip and the test agent IL2 mutein be provided in the mobile phase. In either circumstance, it should be noted that modifications of some proteins for immobilization on a coated SPR chip may interfere with the binding properties of one or both components of the binding pair to be evaluated by SPR. In such cases, it may be necessary to switch the mobile and bound elements of the binding pair or use a chip with a binding agent that facilitates non-interfering conjugation of the protein to be evaluated. In some embodiments, when evaluating the binding affinity of α/β biased hIL2 mutein for a hIL2 receptor subunit using SPR, the a/P biased hIL2 mutein may be derivatized by the C-terminal addition of a poly-His sequence (e.g., 6×His6 or 8×His8) an immobilized on the SPR chip and the hIL2 receptor subunit for which the α/β biased hIL2 mutein's binding affinity is being evaluated is provided in the mobile phase. The means for incorporation of a poly-His sequence into the C-terminus of the α/β biased hIL2 mutein produced by recombinant DNA technology is well known to those of skill in the relevant art of biotechnology. In some embodiments, the binding affinity of a/P biased hIL2 mutein for a hIL2 receptor subunit using SPR substantial accordance with the teaching of Example 7 herein.

In some embodiments, the αβhIL2 mutein muteins useful in the methods of the present disclosure comprise substitutions, deletions, or insertions within the wildtype hIL-2 (wt hIL2) amino acid sequence that modulate the binding of the hIL2 mutein to the extracellular domain of hCD132. The following nomenclature is used herein to refer to substitutions, deletions or insertions. Residues may be designated herein by the one-letter or three-letter amino acid code followed by the IL-2 amino acid position, e.g., “Cys125” or “C125” refers to the cysteine residue at position 125 of SEQ ID NO:4 Substitutions are designated herein by the one letter amino acid code followed by the IL-2 amino acid position followed by the Substituting one letter amino acid code, for example “K35A” refers to a substitution of the lysine (K) residue at position 35 of Sequence ID No. 5 with an alanine (A) residue. A deletion is denoted by “des” followed by the amino acid residue and its position in SEQ ID NO:4. For example the term “des-Alal” or “desA1” refers to the deletion of the alanine at position 1 of the polypeptide of SEQ ID NO:4.

Unless otherwise specified, when reference is made to amino acid substitutions in the human IL2 muteins of the present disclosure, the position of the amino acids is numbered in accordance with hIL2 as used herein refers to the identification of a location of particular amino acid with reference to the position at which that amino acid normally occurs in the sequence of the mature wild type IL2. In some embodiments, the IL2 is hIL2 (SEQ ID NO: 4). For example, in reference to hIL2, “R81” refers to the eighty-first (numbered from the N-terminus) amino acid, arginine, that occurs in sequence of the mature wild type hIL2.

The present disclosure relates to uses of hIL2 muteins which have reduced binding affinity for hCD132 while retaining at least substantially wild-type binding affinity for hCD25 and/or hCD122 (herein referred to as “αβhIL2 mutein”).

In some embodiments, the αβhIL2 muteins useful in the practice of the present disclosure provide modifications that modulate the affinity of the binding of the hIL2 mutein to individual components of the hIL2 receptor (i.e., hCD25, hCD122 and hCD132) as well as combinations of thereof such as hCD122/hCD132 (the “intermediate affinity hIL2 receptor”), hCD25 (the “low affinity IL2 receptor”) and hCD25/hCD122/hCD132 (the “high affinity IL2 receptor”). In some embodiments, the αβhIL2 muteins useful in the practice of the present disclosure have reduced binding affinity for the intermediate affinity hIL2 receptor relative to wt-hIL2.

In some embodiments, the biased hIL2 muteins useful in the methods of the present disclosure comprise substitutions, deletions, or insertions within the wild-type IL-2 amino acid sequence that reduce affinity of the of αβhIL2 mutein to the extracellular domain of hCD132 while retaining significant binding to hCD25.

In some embodiments, the αβhIL2 muteins useful in the methods of the present disclosure comprise substitutions, deletions, or insertions within the wild-type IL-2 amino acid sequence that reduce affinity of the of the αβhIL2 mutein to the extracellular domain of hCD132 while retaining significant binding to hCD122.

In some embodiments, the αβhIL2 muteins useful in the practice of the methods of the present disclosure comprise one or more amino acid substitutions selected from amino acid positions 18, 22, and 126, numbered in accordance with mature wild-type hIL-2.

In some embodiments, the αβhIL2 muteins useful in the practice of the methods of the present disclosure possess decreased binding affinity to the extracellular domain of hCD132 (e.g., <50% the affinity of wild-type hIL2, alternatively <45% the affinity of wild-type hIL2, alternatively <40% the affinity of wild-type hIL2, alternatively <35% the affinity of wild-type hIL2, alternatively <25% the affinity of wild-type hIL2, alternatively <20% the affinity of wild-type hIL2, alternatively <15% the affinity of wild-type IL2, alternatively <10% the affinity of wild-type IL2, or alternatively <5% the affinity of wild-type IL2). In some embodiments, the hIL2 mutein exhibits decreased binding affinity for CD132 relative to wt hIL2 and demonstrates increased binding affinity for CD122 in the presence of CD25, membrane bound CD25 or sCD25, comparable to or greater than wt hIL2. In some embodiments, αβhIL2 muteins of the present disclosure comprise one or more amino acid substitutions that decrease CD132 receptor binding. In some embodiments, the one or more amino acid substitutions that decrease CD132 receptor binding affinity are selected from those amino acids that are at the interface between hIL2 and hCD132. The crystal structure of hIL2 and its interface with hCD132 has been published and other studies have been conducted which have identified those positions of the hIL2 molecule which have been identified as interacting with binding of hIL2 to CD132 include residues L18, Q22, Q126, T123, S127, 1129 and S130. The αβhIL2 muteins useful in the practice of the methods of the present disclosure comprise amino acid substitutions or deletions and one or more of include residues L18, Q22, Q126, T123, S127, 1129 and S130.

Garcia, et al. (International Application Number PCT/2018/062122, PCT International Publication No. WO 2019/104092 A1 published May 31, 2019, hereinafter “Garcia '092”) describes certain IL2 muteins having modifications including positions 18, 22 and 126 that, among other things, exhibit diminished binding for CD132 while retaining partial IL2 activity that are useful in the practice of the presently described methods.

In some embodiments, the αβhIL2 muteins useful in the practice of the methods of the present disclosure possess decreased binding affinity to the extracellular domain of hCD132 (e.g., <50% the affinity of wild-type hIL2, alternatively <45% the affinity of wild-type hIL2, alternatively <40% the affinity of wild-type hIL2, alternatively <35% the affinity of wild-type hIL2, alternatively <25% the affinity of wild-type hIL2, alternatively <20% the affinity of wild-type hIL2, alternatively <15% the affinity of wild-type IL2, alternatively <10% the affinity of wild-type IL2, or alternatively <5% the affinity of wild-type IL2) while retaining substantial affinity (e.g., 20% the affinity of wild-type hIL2, alternatively >30% the affinity of wild-type hIL2, alternatively >40%, alternatively >50% the affinity of wild-type hIL2, alternatively >60% the affinity of wild-type hIL2, alternatively >65% the affinity of wild-type hIL2, alternatively >70% the affinity of wild-type hIL2, alternatively >75% the affinity of wild-type hIL2, alternatively >80% the affinity of wild-type hIL2, alternatively >85% the affinity of wild-type hIL2, alternatively >90% the affinity of wild-type IL2, alternatively >90% the affinity of wild-type IL2, alternatively >95% the affinity of wild-type IL2, alternatively >100% the affinity of wild-type IL2, alternatively >105% the affinity of wild-type hIL2, alternatively >110% the affinity of wild-type IL2, alternatively >115% the affinity of wild-type hIL2, alternatively >125% the affinity of wild-type IL2, or alternatively >150% the affinity of wild-type hIL2) binding affinity for the extracellular domain of the wild-type human CD122 receptor.

In some embodiments, the αβhIL2 muteins useful in the practice of the methods of the present disclosure has reduced binding affinity for the extracellular domain of the hCD132 receptor further includes 1, 2, 3, 4, 5, 6, 7, 8, 9, or 10 or more mutations that increase affinity for the extracellular domain of the wild-type human CD122 receptor. In certain embodiments, the subject hIL-2 mutein useful in the practice of the methods of the present disclosure includes at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) relative to a wild-type IL-2 (e.g., SEQ ID NO:4) and binds the CD122 with higher affinity than a wild-type IL-2. In certain embodiments, the IL-2 mutein binds CD122 with an affinity that is at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% greater than wild-type hIL-2. The binding affinity of hIL-2 mutein can also be expressed as 1.2, 1.4, 1.5, 2, 5, 10, 15, 20, 25, 50, 100, 200, 250 or more-fold increased affinity for the extracellular domain of hCD122 than wild-type hIL-2.

In some embodiments, the αβhIL2 mutein: (a) possesses more than 10% but less than about 90%, alternatively more than 10% but less than less than about 80%, alternatively more than 10% but less than less than about 70%, alternatively more than 10% but less than less than about 60%, alternatively more than 10% but less than less than about 50%, alternatively more than 10% but less than less than about 40%, or alternatively more than 5% but less than less than about 40% binding affinity to the extracellular domain of hCD132 relative to wild-type hIL2; (b) possesses greater than 50%, alternatively greater than 60%, alternatively greater than 70%, alternatively greater than 80%, alternatively greater than 90%, alternatively greater than 100%, alternatively greater than 120%, alternatively greater than 150%, or alternatively greater than 200% binding affinity for the extracellular domain of hCD122 relative to wild-type hIL2; and (c) possesses greater than 50%, alternatively greater than 60%, alternatively greater than 70%, alternatively greater than 80%, alternatively greater than 90%, alternatively greater than 100%, alternatively greater than 120%, or alternatively greater than 150% binding affinity for the extracellular domain of hCD122 relative to wild-type hIL2.

In some embodiments, the αβhIL2 mutein: (a) possesses more than 10% but less than about 90%, alternatively more than 10% but less than less than about 80%, alternatively more than 10% but less than less than about 70%, alternatively more than 10% but less than less than about 60%, alternatively more than 10% but less than less than about 50%, alternatively more than 10% but less than less than about 40%, or alternatively more than 5% but less than less than about 40% binding affinity to the extracellular domain of hCD132 relative to wild-type hIL2; (b) possesses greater than 50%, alternatively greater than 60%, alternatively greater than 70%, alternatively greater than 80%, alternatively greater than 90%, alternatively greater than 100%, alternatively greater than 120%, alternatively greater than 150%, or alternatively greater than 200% binding affinity for the extracellular domain of hCD122 relative to wild-type hIL2; (c) possesses greater than 50%, alternatively greater than 60%, alternatively greater than 70%, alternatively greater than 80%, alternatively greater than 90%, alternatively greater than 100%, alternatively greater than 120%, or alternatively greater than 150% binding affinity for the extracellular domain of hCD122 relative to wild-type hIL2; and (d) the amino acid sequence of the biased IL2 mutein is greater than 70%, alternatively greater than 80%, alternatively greater than 90%, alternatively greater than 95% identical to the amino acid sequence of wild-type hIL2.

In some embodiments, the αβhIL2 muteins useful in the practice of the methods of the present disclosure possess decreased binding affinity to the extracellular domain of hCD132 (e.g., <50% the affinity of wild-type hIL2, alternatively <45% the affinity of wild-type hIL2, alternatively <40% the affinity of wild-type hIL2, alternatively <35% the affinity of wild-type hIL2, alternatively <25% the affinity of wild-type hIL2, alternatively <20% the affinity of wild-type hIL2, alternatively <15% the affinity of wild-type hIL2, alternatively <10% the affinity of wild-type hIL2, or alternatively <5% the affinity of wild-type hIL2) while retaining substantial affinity (e.g, >50% the affinity of wild-type hIL2, alternatively >60% the affinity of wild-type hIL2, alternatively >65% the affinity of wild-type hIL2, alternatively >70% the affinity of wild-type hIL2, alternatively >75% the affinity of wild-type hIL2, alternatively >80% the affinity of wild-type hIL2, alternatively >85% the affinity of wild-type hIL2, alternatively >90% the affinity of wild-type hIL2, alternatively >90% the affinity of wild-type hIL2, alternatively >95% the affinity of wild-type hIL2, alternatively >100% the affinity of wild-type hIL2, alternatively >105% the affinity of wild-type hIL2, alternatively >110% the affinity of wild-type hIL2, alternatively >115% the affinity of wild-type hIL2, alternatively >125% the affinity of wild-type hIL2, or alternatively >150% the affinity of wild-type IL2) for the hCD25/hCD122 receptor complex. In certain embodiments, the αβhIL2 muteins of the present disclosure possess reduced affinity for CD132. In some embodiments, such αβhIL2 muteins incorporate modifications to the primary structure of the wild-type IL2 incorporating one or more modifications at positions 18, 22, and 126 numbered in accordance with wild-type hIL2.

In some embodiments, the αβhIL2 muteins useful in the practice of the methods of the present disclosure possess decreased binding affinity to CD132 while retaining substantial affinity (e.g. >50% the affinity of wild-type hIL2, alternatively >60% the affinity of wild-type hIL2, alternatively >65% the affinity of wild-type hIL2, alternatively >70% the affinity of wild-type hIL2, alternatively >75% the affinity of wild-type hIL2, alternatively >80% the affinity of wild-type hIL2, alternatively >85% the affinity of wild-type hIL2, alternatively >90% the affinity of wild-type hIL2, alternatively >90% the affinity of wild-type IL2, alternatively >95% the affinity of wild-type hIL2, alternatively >100% the affinity of wild-type IL2, alternatively >105% the affinity of wild-type hIL2, alternatively >110% the affinity of wild-type hIL2, alternatively >115% the affinity of wild-type hIL2, alternatively >125% the affinity of wild-type hIL2, alternatively >150% the affinity of wild-type hIL2, alternatively >200% the affinity of wild-type hIL2, alternatively >300% the affinity of wild-type IL2, alternatively >400% the affinity of wild-type hIL2, alternatively >500% the affinity of wild-type IL2) binding affinity for hCD25.

In some embodiments, the αβhIL2 mutein: (a) possesses more than 10% but less than about 90%, alternatively more than 10% but less than less than about 80%, alternatively more than 10% but less than less than about 70%, alternatively more than 10% but less than less than about 60%, alternatively more than 10% but less than less than about 50%, alternatively more than 10% but less than less than about 40%, or alternatively more than 5% but less than less than about 40% binding affinity to the extracellular domain of hCD132 relative to wild-type hIL2; (b) possesses greater than 50%, alternatively greater than 60%, alternatively greater than 70%, alternatively greater than 80%, alternatively greater than 90%, alternatively greater than 100%, alternatively greater than 120%, alternatively greater than 150%, or alternatively greater than 200% binding affinity for the extracellular domain of hCD122 relative to wild-type hIL2; and (c) possesses greater than 50%, alternatively greater than 60%, alternatively greater than 70%, alternatively greater than 80%, alternatively greater than 90%, alternatively greater than 100%, alternatively greater than 120%, or alternatively greater than 150% binding affinity for the extracellular domain of hCD25 relative to wild-type hIL2.

In some embodiments, the αβhIL2 mutein: (a) possesses more than 10% but less than about 90%, alternatively more than 10% but less than less than about 80%, alternatively more than 10% but less than less than about 70%, alternatively more than 10% but less than less than about 60%, alternatively more than 10% but less than less than about 50%, alternatively more than 10% but less than less than about 40%, or alternatively more than 5% but less than less than about 40% binding affinity to the extracellular domain of hCD132 relative to wild-type hIL2; (b) possesses greater than 50%, alternatively greater than 60%, alternatively greater than 70%, alternatively greater than 80%, alternatively greater than 90%, alternatively greater than 100%, alternatively greater than 120%, alternatively greater than 150%, or alternatively greater than 200% binding affinity for the extracellular domain of hCD122 relative to wild-type hIL2; (c) possesses greater than 50%, alternatively greater than 60%, alternatively greater than 70%, alternatively greater than 80%, alternatively greater than 90%, alternatively greater than 100%, alternatively greater than 120%, or alternatively greater than 150% binding affinity for the extracellular domain of hCD25 relative to wild-type hIL2; and (d) the amino acid sequence of the biased IL2 mutein is greater than 70%, alternatively greater than 80%, alternatively greater than 90%, alternatively greater than 95% identical to the amino acid sequence of wild-type hIL2.

In certain embodiments, the αβhIL2 muteins useful in the practice of the methods of the present disclosure disrupt the association of the CD122 with the CD132 (i.e., the formation of the intermediate affinity IL2 receptor complex) such that this CD122/CD132 interaction is reduced by about 2%, about 5%, about 10%, about 15%, about 20%, about 50%, about 75%, about 90%, about 95% or more relative to wild-type hIL-2. In some embodiments, the one or more mutations reducing the binding affinity of αβhIL2 mutein for CD132 is an amino acid substitution. In some embodiments, the subject αβhIL2 mutein consists of 1, 2, 3, 4, 5, 6, 7, 8, 9, 10, 11, 12, 13, 14, or 15 amino acid substitutions as compared to a wild-type IL-2 (SEQ ID NO: 4).

In certain embodiments, the αβhIL2 muteins useful in the practice of the methods of the present disclosure disrupt the association of the hCD25/hCD122 complex with hCD132 such that this hCD25/hCD122 interaction with hCD132 (i.e., the formation of the high affinity IL2 receptor complex) is reduced by about 2%, about 5%, about 10%, about 15%, about 20%, about 50%, about 75%, about 90%, about 95% or more relative to wild-type hIL-2. In some embodiments, the one or more mutations reducing the binding affinity of the αβhIL2 muteins for hCD132 is an amino acid substitution. In some embodiments, the subject αβhIL2 muteins consists of 1, 2, 3, 4, 5, 6, 7, 8, 9, 10, 11, 12, 13, 14, or 15 amino acid substitutions as compared to a wild-type IL-2 (SEQ ID NO: 4).

In some embodiments, the αβhIL2 muteins useful in the practice of the methods of the present disclosure are partial agonists having a reduced capability to stimulate signaling in a CD25neg cell as compared to wild-type hIL-2. In some embodiments, the αβhIL2 mutein stimulates pERK1/ERK2 signaling in an CD25neg cell at a level that is 1%, 5%, 10%, 15%, 20%, 25%, 30%, 35%, 40%, 45%, 50%, 55%, 60%, 65%, 70%, 75%, 80%, 85%, 90%, 95% or less of the level that wild-type IL-2 stimulates pERK1/ERK2 signaling in the same cell. In some embodiments, the CD25neg cell is a T cell. In some embodiments, the CD25neg T cell is a CD8+ T cell. In other embodiments, the CD8+ T cell is an activated CD25neg CD8+ T cell. In some embodiments, the CD25neg cell is a natural killer (NK) cell. STAT5 and ERK1/2 signaling can be measured, for example, by phosphorylation of STAT5 and ERK1/2 using any suitable method known in the art.

In certain embodiments, the αβhIL2 muteins useful in the practice of the methods of the present disclosure is a partial agonist having diminished ability to induce lymphocyte proliferation of a CD25^{neg} cell as compared to wild-type hIL-2. In some embodiments, the CD25^{neg} cell is a natural killer (NK) cell.

In some embodiments, the αβhIL2 muteins useful in the practice of the methods of the present disclosure that are partial agonists have one or more reduced functions as compared to wild-type IL-2.

In some embodiments, the αβhIL2 muteins useful in the practice of the methods of the present disclosure are partial agonists. In certain embodiments, the αβhIL2 muteins useful in the practice of the methods of the present disclosure is a partial agonist having reduced capabilities to stimulate one or more signaling pathways that are dependent on CD122/CD132 heterodimerization.

In some embodiments, the αβhIL2 muteins have a reduced capability to stimulate phosphorylation in an CD122+ cell as compared to wild-type hIL-2. In some embodiments, the αβhIL2 muteins stimulate STAT5 phosphorylation in an IL-2R+ cell at a level that is 1%, 5%, 10%, 15%, 20%, 25%, 30%, 35%, 40%, 45%, 50%, 55%, 60%, 65%, 70%, 75%, 80%, 85%, 90%, 95% or less of the level that wild-type IL-2 stimulates STAT5 phosphorylation in the same cell.

In some embodiments, the αβhIL2 muteins useful in the practice of the methods of the present disclosure are full agonists.

In some embodiments, the αβhIL2 muteins useful in the practice of the methods of the present disclosure are super agonists.

In some embodiments, the one or more amino acid substitutions that provide decreased binding affinity of the αβhIL2 mutein for the ECD of hCD132 receptor subunit are selected from those amino acids that are at the interface between hIL2 and the ECD of hCD132. The crystal structure of hIL2 and its interface with the ECD of hCD132 has been published and other studies have been conducted which have identified residues L18, Q22, Q126, T123, S127, 1129 and S130 residues of the wt-hIL2 as involved in the binding of wt-hIL2 to the ECD of hCD132. In some embodiments, the αβhIL2 mutein comprises amino acid substitutions at positions 18, 22 and/or 126 numbered in accordance with wt-hIL2. As noted above, the numbering of residues in the αβhIL2 muteins of the present disclosure is in accordance with the numbering of the numbering of the residues in the mature (lacking the signal peptide) form of wild-type human IL2 (SEQ ID NO:4).

In some embodiments, the amino acid substitutions at residue L18 of an αβhIL2 mutein are selected from the group consisting of L18R, L18G, L18M, L18F, L18E, L18H, L18W, L18K, L18Q, L18S, L18V, L18I, L18Y, L18H, L18D, L18N and L18T.

In some embodiments, the amino acid substitutions at residue Q22 of an αβhIL2 mutein are selected from the group consisting of Q22F, Q22E, Q22G, Q22A, Q22L, Q22M, Q22F, Q22W, Q22K, Q22S, Q22V, Q22I, Q22Y, Q22H, Q22R, Q22N, Q22D, Q22T, and F.

In some embodiments, the amino acid substitutions at residue Q126 of an αβhIL2 mutein are selected from the group consisting of Q126H, Q126M, Q126K, Q126C, Q126D, Q126E, Q126G, Q126I, Q126R, Q126S, or Q126T.

In some embodiments, an αβhIL2 mutein comprises a substitution at residue S130 selected from the group consisting of S130R and S130G.

In some embodiments, the αβhIL2 mutein is an hIL2 mutein comprising the following mutations at positions 18, 22, and 126 wherein:
the leucine at position 18 (L18) is substituted with an amino acid selected from the group consisting of R, L, G, M, F, E, H, W, K, Q, S, V, I, Y, H, D and T;
the glutamine at position 22 (Q22) is substituted with an amino acid selected from the group consisting of E, G, A, L, M, F, W, K, S, V, I, Y, H, R, N, D, T, and F;
and
the glutamine at position 126 (Q126) is substituted with an amino acid selected from the group consisting of H, M, K, C, D, E, G, I, R, S, and T.

In some embodiments, the αβhIL2 mutein is an hIL2 mutein comprising the following mutations at positions 1, 18, 22, and 126 wherein:
the alanine at position 1 (A1) is deleted (des-Alal)
the leucine at position 18 (L18) is substituted with an amino acid selected from the group consisting of R, L, G, M, F, E, H, W, K, Q, S, V, I, Y, H, D and T;
the glutamine at position 22 (Q22) is substituted with an amino acid selected from the group consisting of E, G, A, L, M, F, W, K, S, V, I, Y, H, R, N, D, T, and F; and
the glutamine at position 126 (Q126) is substituted with an amino acid selected from the group consisting of H, M, K, C, D, E, G, I, R, S, and T.

In some embodiments, the αβhIL2 mutein comprises a set of mutations selected from the group consisting of the following sets of mutations: L18R, Q22E, and Q126H; L18R, Q22E, and Q126K; L18R, Q22E and Q126M; L18R, Q22E Q126T; L18R; Q22E; V91K; V91R; Q126H; L18R, and Q126H; Q22E, and Q126H; L18G, Q22E and Q126H; L18A, Q22E and Q126H; L18M, Q22E and Q126H; L18F, Q22E and Q126H; L18W, Q22E and Q126H; L18K, Q22E and Q126H; L18Q, Q22E and Q126H; L18E, Q22E and Q126H; L18S, Q22E and Q126H; L18V, Q22E and Q126H; L181, Q22E and Q126H; L18Y, Q22E and Q126H; L18H, Q22E and Q126H; L18N, Q22E and Q126H; L18D, Q22E and Q126H; L18T, Q22E and Q126H; L18R, Q22G and Q126H; L18R, Q22A and Q126H; L18R, Q22L and Q126H; L18R, Q22M and Q126H; L18R, Q22F and Q126H; L18R, Q22W and Q126H; L18R, Q22K and Q126H; L18R, Q22S and Q126H; L18R, Q22V and Q126H; L18R, Q22I and Q126H; L18R Q22Y and Q126H; L18R Q22H and Q126H; L18R Q22R and Q126H; L18R Q22N and Q126H; L18R Q22D and Q126H; and L18R Q22T and Q126H, in each case optionally further comprising a deletion of the N-terminal alanine residue (des Alal).

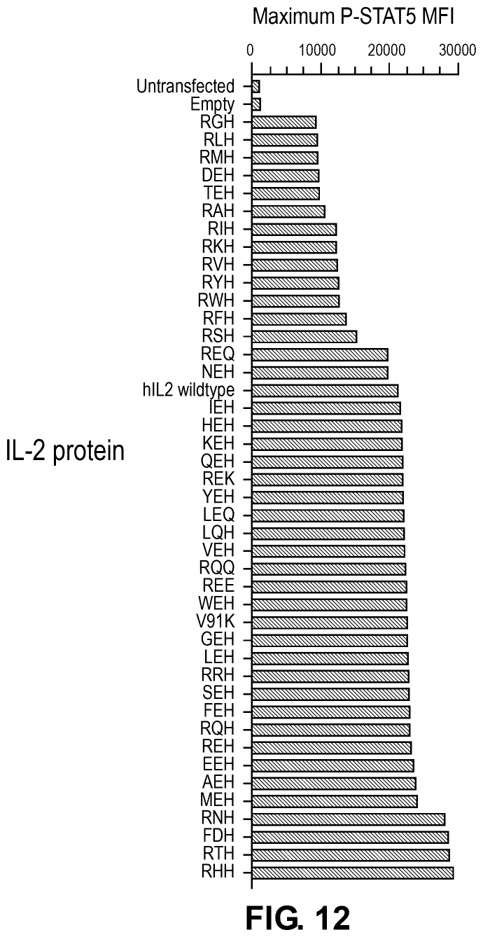

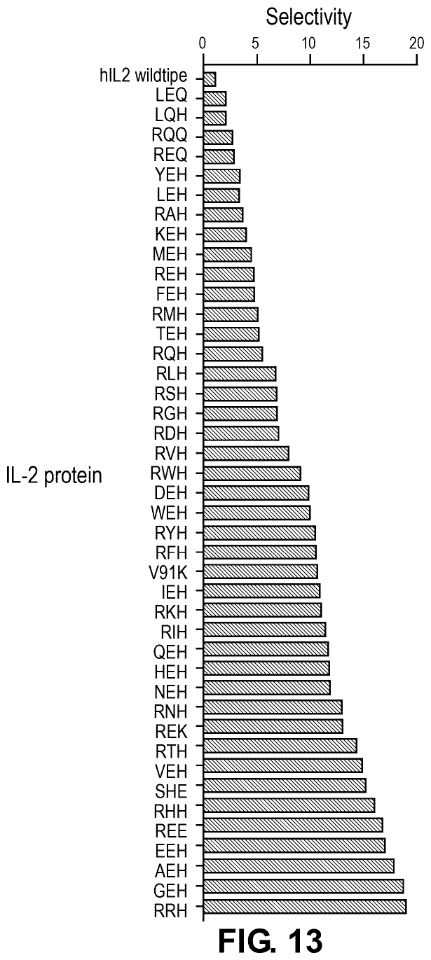

In some embodiments, the αβhIL2 mutein comprises the sets of amino acid substitutions at positions 18, 22, and 126 (numbered in accordance with hIL2) provided in Table 5 below (each row corresponding to set of amino acid substitutions). As a convenient naming convention for these molecules, the molecule is referred to by the amino acids at positions 18, 22, and 126 such that, an hIL2 mutein containing the substitutions L18W, Q22E and Q126H is referred to as “WEH.” These three letter abbreviations are reflected in FIGS. 12 and 13 of the attached drawings.

TABLE 5
α/β-biased hIL2 Substitutions at Positions 18, 22, and 126

| Position 18 | Position 22 | Position 126 |
|---|---|---|
| (wt = L) | (wt = Q) | (wt = Q) |
| R | E | H |
| R | E | K |
| R | E | M |
| R | E | T |
| R | Q | Q |
| L | E | Q |
| L | Q | H |
| R | Q | H |
| L | E | H |
| G | E | H |
| A | E | H |
| M | E | H |
| F | E | H |
| W | E | H |
| K | E | H |
| Q | E | H |
| E | E | H |
| S | E | H |
| V | E | H |
| I | E | H |
| Y | E | H |
| H | E | H |
| N | E | H |
| D | E | H |
| T | E | H |
| R | G | H |
| R | A | H |
| R | L | H |
| R | M | H |
| R | F | H |
| R | W | H |
| R | K | H |
| R | S | H |
| R | V | H |
| R | I | H |
| R | Y | H |
| R | H | H |
| R | R | H |
| R | N | H |
| R | D | H |
| R | T | H |

When wt hIL2 is expressed endogenously in mammalian cells, the hIL2 is expressed as a pre-protein comprising a signal peptide which is efficiently cleaved in mammalian cells resulting in the N-terminal amino acid of the mature hIL2 polypeptide being an alanine residue (Alal). While expression of the αβhIL2 mutein in mammalian cells is possible, it typically more expensive than bacterial cell production and expression in mammalian cells may also result in non-natural glycosylation of the αβhIL2 mutein depending on the cell line used. Consequently, production of the αβhIL2 mutein in bacterial cells may preferred in certain circumstances. However, direct expression (i.e., not as a fusion protein) of a αβhIL2 mutein in bacterial cells results in the addition of a N-terminal methionine residue. If the Ala1 characteristic of the wt IL2 sequence is retained in the αβhIL2 mutein, this results in a proline at the +2 position relative to N-terminal methionine. When a proline is present at the +2 position relative to the N-terminal methionine, the endogenous bacterial methionyl amino peptidase (MAP) of the bacterial host cell does not efficiently cleave the N terminal methionine. Consequently, bacterial direct expression of the αβhIL2 mutein will typically result in a mixture of αβhIL2 mutein species, one fraction having an N-terminal and another species lacking the N-terminal methionine. Such a mixture of IL2 species is difficult to resolve by typical manufacturing procedures which results in increased processing, loss of product and creates difficulties when attempting to conjugate the molecules to N-terminus of the αβhIL2 mutein such as a targeting molecules or carrier molecules such as PEG molecule. However, by deleting Ala1 from the αβhIL2 mutein, the residue in the +2 position relative to the N-terminal methionine is a threonine (T3) which results in very efficient cleavage of the N-terminal methionine and facilitates bacterial production of the IL2 mutein and provides a more uniform αβhIL2 mutein product. In some embodiments, the present disclosure, provides hIL2 muteins comprising a deletion of the alanine at position 1 (des-Alal; des-Al numbered in accordance with hIL2).

A series of exemplary hIL2 muteins comprising the amino acid substitutions at positions 18, 22 and/or 126 which interface with CD132 as described in Table 5 were prepared and tested for IL2 activity and selectivity with respect to CD25+ and CD25− T cells. The molecules were prepared and tested in substantial accordance with the teaching of the Examples herein. Briefly, nucleic acid sequences encoding the various When expressed in Expi293 cells, human IL-2 muteins, human IL-2 REK, mouse IL-2-REH (L18R, Q22E, Q126H), CD25(22-240) and CD122(27-240) were purified via Ni-Excel (Cytiva) affinity chromatography. Supes were supplemented with 5 mM Imidazole, while wash and elution were performed in PBS supplemented with 30 and 250 mM Imidazole, respectively. Affinity elutions were further purified via preparative Size Exclusion Chromatography (SEC) on HiLoad 16/600 Superdex 200 pg column equilibrated in PBS buffer. Purity was established via reducing 4-20% Tris-glycine SDS-PAGE (Biorad) and SEC-MALS (Wyatt) performed on Superdex Increase 10/300 GL column equilibrated in PSB.

To demonstrate the activity of the hIL2 muteins having decreased binding affinity for CD132 relative to wild-type hIL2 of the present disclosure and their preferential activation of CD25 expressing cells, a series of hIL2 muteins were prepared and evaluated for their ability to provide selective activation of YT cells, an NK cell expressing the intermediate affinity dimeric form of the IL2 receptor and a YT cell variant referred as YT CD25 which is a YT cell that has been modified to express CD25 on its surface (iCD25+) resulting in a human immune cell that expresses all three components of the high affinity trimeric IL2 receptor.

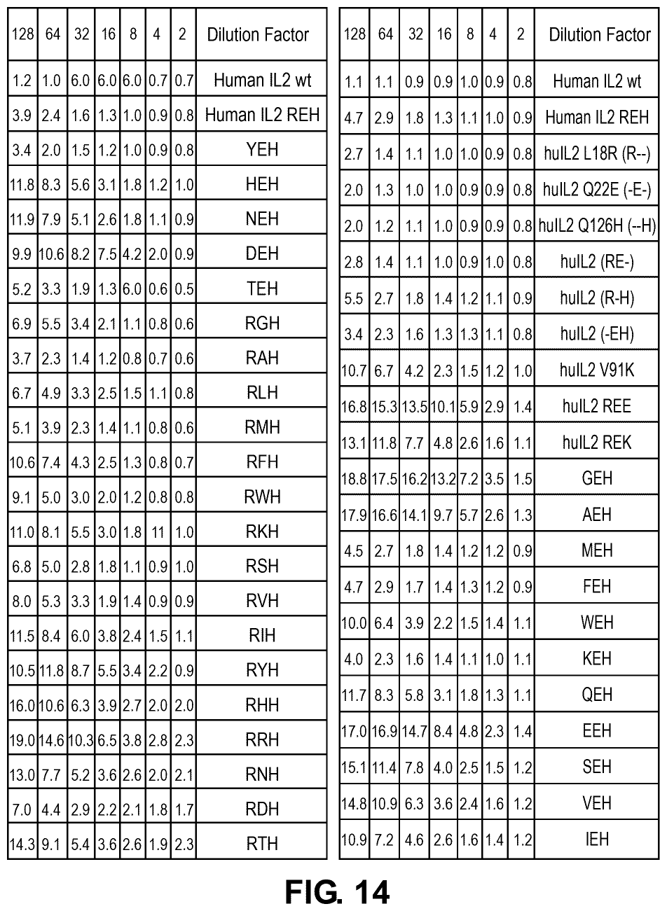

The results of these experiments are provided in FIGS. 12, 13 and 14 of the attached drawings. As illustrated in FIG. 12, the hIL2 muteins comprising amino acid substitutions involved in the binding of hIL2 to hCD132 at positions 18, 22 and/or 126 demonstrated significant increases in pSTAT5 signaling demonstrating in YT CD25 cells that the hIL2 muteins retain significant hIL2 activity relative to wt hIL2. As illustrated in FIG. 2, hIL2 muteins of the present disclosure demonstrated preferential pSTAT5 signaling activity relative to wild type hIL2 on CD25 positive YT CD25 cells relative to the CD25 negative YT cells. The data from the dilution of these molecules is provided in FIG. 3 of the attached drawings.

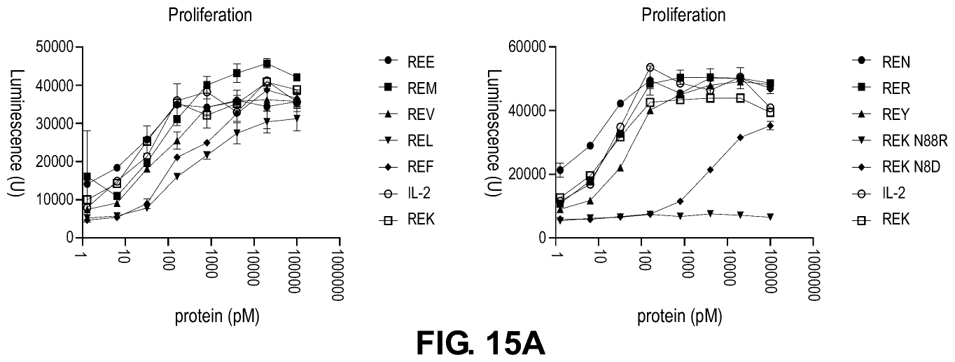

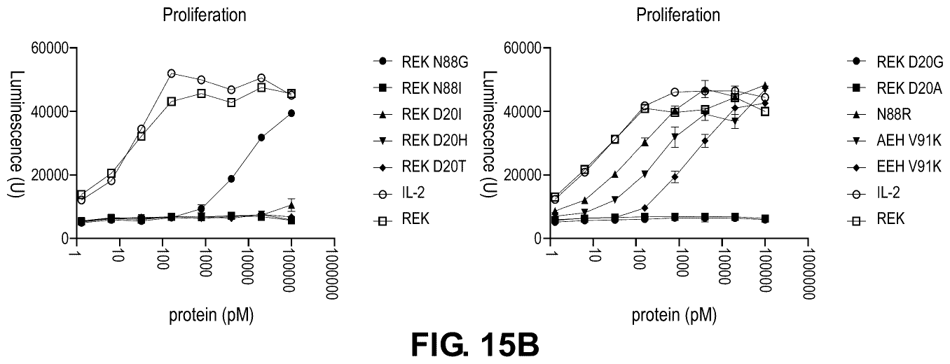

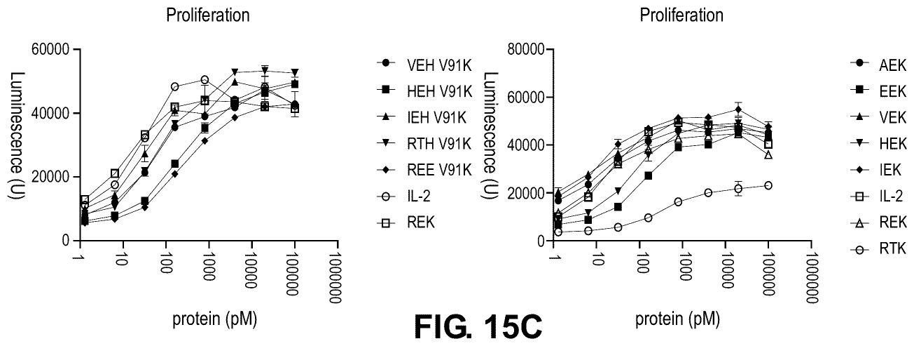

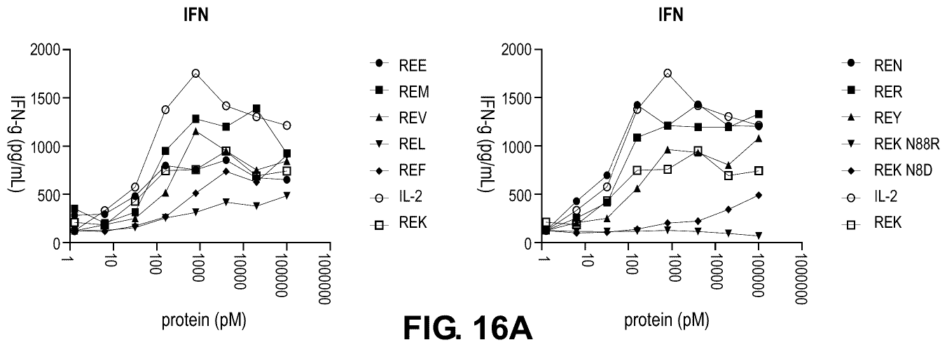

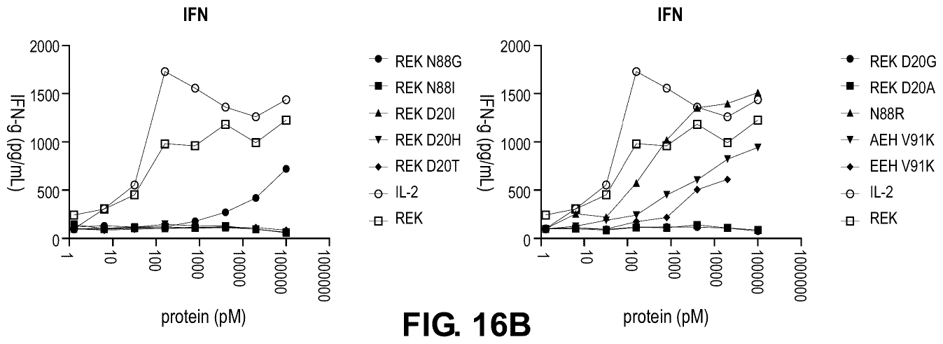

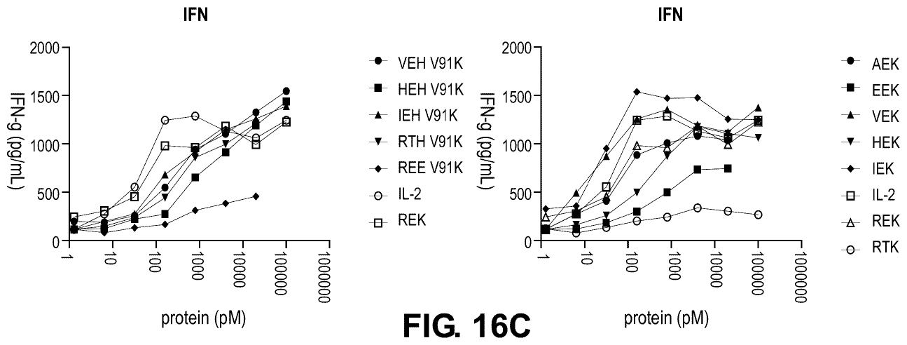

An additional study was conducted to evaluate αβhIL2 muteins of the present disclosure for activity in CD4 positive human T cells, 3F8 cells. The 3F8 cell line was generated by activation of PBMCs obtained from a healthy human donor with the EBV transformed B cell line JY. The CD4 positive T cell clone 3F8 expresses CD25 and CD122 and proliferates and produces IFNγ in response to IL-2. Additional representative αβhIL2 muteins as detailed in Table 6 below were evaluated for proliferative activity and IFNγ production in 3F8 cells accordance with the teaching of Example 8 herein. The data from this experiment is provided in Table 6 below and FIG. 15 (cell proliferation) and FIG. 16 (IFNγ production) of the attached drawings. The IC₅₀ is corrected for the protein concentration in the transfection supernatant.

TABLE 6
Proliferation and IFNγ Production by Human CD4
Positive T Cell Clone 3F8 In Response to hIL2 Muteins

| | Proliferation | IFNγ Production |
|---|---|---|
| Construct | IC₅₀ (pM) | IC₅₀ (pM) |
| IL-2 | 30.7 | 19.7 |
| REK | 14.2 | 17.7 |
| REE | 33.0 | 18.4 |
| REM | 32.6 | 12.7 |
| REV | 20.8 | 21.2 |
| REL | 68.4 | 33.8 |
| REF | 37.6 | 38.3 |
| REN | 13.7 | 15.7 |
| RER | 13.1 | 13.1 |
| REY | 19.3 | 22.1 |
| AEK | 13.7 | 19.0 |
| EEK | 36.0 | 58.7 |
| VEK | 15.5 | 4.6 |
| HEK | 20.9 | 30.4 |
| IEK | 10.0 | 8.8 |
| RTK | 62.8 | NA |

The foregoing data in Table 6 and FIGS. 15 and 16 demonstrate that the αβhIL2 muteins of the present disclosure having decreased binding affinity for CD132 relative to wt hIL2 and are effective in stimulating the proliferation of and production of IFNγ from CD25+/CD122+ human immune cells.

In addition to those modifications incorporated into the αβhIL2 muteins that modulating the binding of hIL2 to hCD132, the αβhIL2 muteins of the present disclosure may optionally further comprise one or more amino acid substitutions or deletions that confer additional beneficial properties on the αβhIL2 mutein as described in more detail below.

In some embodiments, the αβhIL2 muteins comprise one or more mutations in positions of the hIL-2 sequence that either contact CD25 or alter the orientation of other positions contacting CD25 resulting in an hIL2 mutein possessing increased affinity for CD25. In some embodiments, the αβhIL2 muteins of the present disclosure comprise one or more the substitutions V69A and Q74P which have been described as increasing the binding affinity of hIL2 for CD25.

In addition to the modifications to the amino acid sequence of the αβhIL2 mutein to provide reduced binding to CD132 and optionally provide increased binding to CD25 and/or CD122, the αβhIL2 mutein of the present disclosure may further comprise one more conservative amino acid substitution within the amino acid sequence of the αβhIL2 mutein which substitution does not result in substantial alteration of the activity profile of the αβhIL2 mutein. Such conservative substitutions include those described by Dayhoff in The Atlas of Protein Sequence and Structure 5 (1978), and by Argos in EMBO J., 8:779-785 (1989). Conservative substitutions are generally made in accordance with the following Table 7.

TABLE 7
Exemplary Conservative Amino Acid Substitutions

| Wild-type Residue | Substitution(s) |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu, Met, Leu, Ile |
| Phe | Met, Leu, Tyr, Trp |
| Ser | Thr |
| Thr | Ser |
| Trp | Tyr, Phe |
| Tyr | Trp, Phe |
| Val | Ile, Leu |

In some embodiments, the αβhIL2 muteins of the present disclosure comprise one or more amino acid substitutions that increase hCD122 receptor binding (or binding to the ECD of hCD122). In some embodiments, the αβhIL2 muteins useful in the practice of the methods of the present disclosure having a reduced binding affinity for CD132 receptor further includes 1, 2, 3, 4, 5, 6, 7, 8, 9, or 10 or more mutations that increase CD122 binding affinity. In certain embodiments, αβhIL2 muteins useful in the practice of the methods of the present disclosure include at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) relative to wt hIL2 such that the hIL2 mutein binds the CD122 with higher affinity than wt hIL2. In certain embodiments, the hIL2 mutein binds CD122 with an affinity that is at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% greater than wild type IL2. The binding affinity of the αβhIL2 muteins can also be expressed as 1.2, 1.4, 1.5, 2, 5, 10, 15, 20, 25, 50, 100, 200, 250 or more fold greater affinity for the CD122 than wt hIL2.

In some embodiments, the αβhIL2 mutein comprises the one or more amino acid substitutions that increase hCD122 receptor binding affinity are selected from those amino acids that are at the interface between hIL2 and hCD122. Based on the crystal structure of hIL2 with its receptor, those positions which have been identified as interacting with binding of hIL2 to hCD122 include but are not limited to Q74, L80, R81, L85, 186, I89V, and 192 numbered in accordance with mature wt hIL2. In some embodiments, the αβhIL2 mutein comprises one or more amino acid substitutions that enhance CD122 binding affinity including but not limited the group consisting of Q74N, Q74H, Q74S, L80F, L80V, R81D, R81T, L85V, I86V, I89V, and/or I92F or combinations thereof. In some embodiments, the αβhIL2 mutein comprises the amino acid substitutions L80F, R81D, L85V, I86V and I92F. In some embodiments, the αβhIL2 mutein comprises the amino acid substitutions N74Q, L80F, R81D, L85V, 186V, 189V, and 192F.

In one aspect, the present disclosure provides αβhIL2 muteins exhibiting significant or enhanced binding affinity for hCD25 and reduced binding affinity for hCD132 (or the extracellular domain of hCD132) receptor as compared to wild type human IL2 (hIL2). In some embodiments, the αβhIL2 muteins of the present disclosure comprise one or more amino acid substitutions that increase hCD25 binding. In some embodiments, the one or more amino acid substitutions to increase hCD25 receptor binding affinity are selected from those amino acids that are at the interface between hIL2 and hCD25. In some embodiments, the αβhIL2 muteins comprise one or more mutations in positions of the IL2 sequence that either contact CD25 or alter the orientation of other positions contacting CD25 resulting in an αβhIL2 mutein possessing increased affinity for CD25. Based on the crystal structure of hIL2 with its receptor and other studies, those positions which have been identified as interacting with binding of hIL2 to hCD25 include V69 and Q74, numbered in accordance with mature wt hIL2. In some embodiments, the αβhIL2 muteins of the present disclosure comprise one or more the substitutions V69A and Q74P.

The αβhIL2 muteins of the present disclosure may comprises modifications to eliminate the O-glycosylation site at position Thr3 (T3) to facilitate the production of an a-glycosylated hIL2 mutein when the IL2 mutein is expressed in a eucaryotic expression system, particularly in mammalian host cells such as CHO or HEK cells. In one embodiment, the αβhIL2 mutein of the present disclosure comprises an amino acid modification, deletion or substitution at position Thr3 (T3) to prevent the O-glycosylation at T3. U.S. Pat. No. 5,116,943; Weiger et al., (1989) Eur. J. Biochem., 180:295-300. In one embodiment, the modification at T3 is an amino acid substitution. In some embodiments, the αβhIL2 muteins of the present disclosure may comprise an amino acid substitution at T3 selected from the amino acid substitutions include T3A, T3G, T3Q, T3E, T3N, T3D, T3R, T3K, and T3P which removes the glycosylation site at position 3 without eliminating biological activity. In one embodiment, the αβhIL2 mutein of the present disclosure comprises the amino acid substitution T3A. In some embodiments the T3 residue may be substituted with a cysteine residue (T3S) to facilitate for selective N-terminal modification, especially PEGylation of the sulfhydryl group of the cysteine (See, e.g. Katre, et al. U.S. Pat. No. 5,206,344 issued Apr. 27, 1993).

In some embodiments of the disclosure, αβhIL2 muteins of the present disclosure comprise amino acid substitutions to avoid vascular leak syndrome, a substantial negative and dose limiting side effect of the use of IL2 therapy in human beings without out substantial loss of efficacy. See, Epstein, et al., U.S. Pat. No. 7,514,073B2 issued Apr. 7, 2009. In some embodiments, the αβhIL2 muteins of the present disclosure further comprise on or more amino acid substitutions selected from the group consisting of R38W, R38G, R39L, R39V, F42K and H55Y.

In some embodiments of the disclosure, αβhIL2 muteins of the present disclosure may optionally comprise an amino acid substitution of the methionine 104, in some instances with an alanine residue (M104A). Elimination of the methionine at position 104 provides a αβhIL2 mutein having improved resistance to oxidation and loss of activity. Koths, et al. U.S. Pat. No. 4,752,585 issued Jun. 21, 1988.

The wt hIL2 sequence comprises an unpaired cysteine residue at position 125. Unpaired cysteines present the opportunity for misfolding of the protein by incorrect disulfide bridges between cysteine sulfhydryl groups. This may be a particular issue when the αβhIL2 mutein is to be expressed recombinantly in bacteria and isolated from inclusion bodies. Consequently, the αβhIL2 muteins of the present disclosure may optionally comprise an amino acid substitution at position 125. In some embodiments, the αβhIL2 muteins of the present disclosure may optionally comprise a C125A or C125S amino acid substitution.

In some embodiments, the αβhIL2 muteins useful in the practice of the methods of the present disclosure comprise an amino acid substitution at position 91. In some embodiments, αβhIL2 mutein comprises a substitution at position 91 selected from the substitutions V91K, V91R, V91K. In some embodiments, the αβhIL2 muteins useful in the practice of the methods of the present disclosure comprise an amino acid substitution at position 91 may be presented as an Fc fusion as more fully described in Gavin, et al. U.S. Pat. No. 9,580,486B2 granted Feb. 28, 2017 the teaching of which is herein incorporated by reference with respect to the construction Fc fusions of IL2 muteins comprising a substitution at position 91.

It has been observed that deletion of amino acids at the N-terminus of the hIL2 molecule do not results in substantial loss of IL2 activity. The αβhIL2 muteins of the present disclosure may optionally comprise deletions of N-terminal amino acids at positions 1-9, alternatively positions 1-8, alternatively positions 1-7, alternatively positions 1-6, alternatively positions 1-5, alternatively positions 1-4, alternatively positions 1-3, alternatively positions 1-2 or alternatively positions 1 (des-Alal) while retaining hIL2 activity and reduced binding affinity for CD132 of the αβhIL2 muteins. αβhIL2 muteins may comprise deletion of the alanine at position 1 (desAla1) to facilitate recombinant production of substantially pure αβhIL2 muteins in bacterial expression systems. The αβhIL2 muteins may comprise deletion of positions 1-3 which further eliminates the glycosylation site at T3.

In some embodiments, hIL2 muteins may be affinity matured to enhance their affinity for CD25 and/or CD122 resulting in modifications to the amino acid sequence of the hIL2 mutein. An “affinity matured” polypeptide is one having one or more alteration(s) in one or more residues which results in an improvement in the affinity of the polypeptide for its receptor, or vice versa, compared to a parent polypeptide which does not possess those alteration(s). Affinity maturation can be performed to increase the binding affinity of the IL2 mutein by at least about 10%, alternatively at least about 50%, alternatively at least about 100% alternatively at least about 150%, or from twofold, threefold, fourfold or fivefold as compared to the parent IL2 mutein polypeptide.

One issue associated with the use of wt-hIL2 in therapeutic applications in mammalian subjects is its comparatively short lifetime in the circulation of the subject to be treated, often of the order of minutes or perhaps hours. In some embodiments of the invention, where the αβhIL2 muteins are administered to a mammalian subject, the αβhIL2 mutein is modified to provide for an extended duration of action (e.g. half-life) in a mammalian subject. In some embodiments, the αβhIL2 mutein modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the αβhIL2 mutein to provide an extended duration of action in a mammalian subject include (but are not limited to); amino acid substitutions in the primary sequence of the αβhIL2 muteins, conjugation of the αβhIL2 mutein to one or more carrier molecules, providing αβhIL2 mutein in the form of a fusion protein with additional polypeptide sequences (e.g, αβhIL2 mutein-Fc fusions) and PEGylated αβhIL2 muteins.

It should be noted that the more than one type of modification that provides for an extended duration of action in a mammalian subject may be employed with respect to a given αβhIL2 mutein. For example, the αβhIL2 mutein of the present disclosure may comprise both amino acid substitutions that provide for an extended duration of action as well as conjugation to a carrier molecule such as a polyethylene glycol (PEG) molecule.

In some embodiments, in addition to those amino acid substitutions that result in reduced binding affinity to hCD132 while retaining significant binding affinity for hCD122 and/or hCD25, the primary sequence of the αβhIL2 mutein may modified further modified by incorporation of one or more amino acid substitutions provide an extended duration of action. See, e.g. Dakshinamurthi, et al. (2009) International Journal of Bioinformatics Research 1(2):4-13). Examples of such amino acid substitutions that provide for an extended duration of action are one or more amino acid substitutions selected from the group consisting of one, two or all three of the V91R, K97E and Ti 13N. In some embodiments, in addition to those amino acid substitutions that result in reduced binding affinity to hCD132 while retaining significant binding affinity for hCD122 and/or hCD25, αβhIL2 muteins useful in the practice of the methods of the present disclosure comprise one or more amino acid substitutions selected from the group consisting of V91R, K97E and T113N.

In some embodiments an αβhIL2 mutein having an extended duration of action in a mammalian subject and useful in the practice of the present disclosure is achieved by covalent attachment of the αβhIL2 mutein to one or more carrier molecules. As used herein, the term “carrier molecules” refers to large, slowly metabolized macromolecules. Examples such slowly metabolized macromolecules carriers include proteins; polysaccharides, such as sepharose, agarose, cellulose, or cellulose beads; polymeric amino acids such as polyglutamic acid, or polylysine; amino acid copolymers. In particular embodiments, particularly where it is desirable to induce a host immune response, the αβhIL2 mutein may be conjugated to one or more immunogenic agents such as inactivated virus particles; inactivated bacterial toxins such as toxoid from diphtheria, tetanus, cholera, or leukotoxin molecules; inactivated bacteria, dendritic cells, thyroglobulin; VP6 polypeptides of rotaviruses; influenza virus hemaglutinin, influenza virus nucleoprotein; keyhole limpet hemocyanin (KLH); and hepatitis B virus core protein and surface antigen S.

Examples of protein carrier molecules which may be covalently attached to the αβhIL2 mutein to provide an extended duration of action in vivo include, but are not limited to albumins, antibodies and antibody fragments such and Fc domains of IgG molecules

In some embodiments, the carrier molecule is an albumin molecule. Conjugation of proteins to albumin molecules is known in the art to facilitate extended exposure in vivo. In one embodiment of the invention, the αβhIL2 mutein is conjugated to albumin via chemical linkage or expressed as a fusion protein with an albumin molecule referred to herein as an “αβhIL2 mutein albumin fusion.” The term “albumin” as used in the context αβhIL2 mutein albumin fusions include albumins such as human serum albumin (HSA), cyno serum albumin, and bovine serum albumin (BSA). In some embodiments, the HSA the HSA comprises a C34S or K573P amino acid substitution relative to the wild-type HSA sequence According to the present disclosure, albumin can be conjugated to a αβhIL2 mutein at the carboxyl terminus, the amino terminus, both the carboxyl and amino termini, and internally (see, e.g., U.S. Pat. Nos. 5,876,969 and 7,056,701). In the HAS-αβhIL2 mutein conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a αβhIL2 mutein fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the αβhIL2 mutein and the αβhIL2 mutein, the αβhIL2 mutein—albumin complex may be provided as a fusion protein comprising an albumin polypeptide sequence and an αβhIL2 mutein recombinantly expressed in a host cell as a single polypeptide chain, optionally comprising a linker molecule between the albumin and αβhIL2 mutein. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein solated from the host cell culture by techniques well known in the art.

In some embodiments, extended in vivo duration of action of the αβhIL2 mutein may be achieved by conjugation to the Fc domain derived from a mammalian (preferably human) immunoglobulin such as an IgG1 or IgG4 molecule. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. A wide variety of modifications have been introduced into the naturally occurring Fc domain such modified hinge regions, modifications to reduce effector function of the Fc, modifications to facilitate disulfide linkages between the Fc subunits as well as incorporation of amino acid substitutions in Fc monomers to provide geometrically complementary structures enabling consistent 1:1 association of Fc dimer subunits so modified.

The Fc domain of the αβhIL2 mutein-Fc fusion can be a naturally occurring or synthetic polypeptide that is homologous to the IgG C-terminal domain produced by digestion of IgG with papain. IgG Fc has a molecular weight of approximately 50 kDa. The αβhIL2 mutein fusion can include the entire Fc region, or a smaller portion that retains the ability to extend the circulating half-life of a chimeric polypeptide of which it is a part. Typically, the nucleic acid sequence encoding the αβhIL2 mutein is provided in frame to one or both subunits of an Fc domain and expressed as a fusion protein as discussed above. The use of Fc fusions as carrier molecules for heterologous polypeptide sequences is well known in the art and are used in a significant number of approved pharmaceutical biologic agents.

Examples of geometrically complementary Fc monomeric subunits are the “knobs-into-holes” Fc modifications as described in Ridgeway et al. (1996) Protein Eng. 9 617-621 and U.S. Pat. No. 5,731,168, issued Mar. 24, 1998. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. The knob-into-hole format is frequently used to facilitate the expression of a first polypeptide (e.g., an hIL2 mutein) on a first Fc monomer with a “knob” modification and a second polypeptide on the second Fc monomer possessing a “hole” modification to facilitate the expression of heterodimeric polypeptides or bi-specific binding molecules. Engineered Fc domains useful in the preparation of extended duration αβhIL2 muteins may optionally be modified by the introduction of cysteine residues at positions S354 and Y349 which results in a stabilizing disulfide bridge between the two antibody heavy chains in the Fe region (Carter, et al. (2001) Immunol Methods 248, 7-15). Engineered Fc domains useful in the preparation of extended duration αβhIL2 muteins may optionally comprise a mutation that inhibits complement fixation and Fc receptor binding. Engineered Fc domains useful in the preparation of extended duration αβhIL2 muteins may optionally designed to be lytic, i.e., able to bind complement or to lyse cells via another mechanism such as antibody-dependent complement lysis (ADCC).

In some embodiments, extended in vivo duration of action of the αβhIL2 mutein may be achieved by conjugation to one or more polymeric carrier molecules such as XTEN polymers or water soluble polymers.

The αβhIL2 mutein may further comprise an XTEN polymer. The XTEN polymer may be is conjugated (either chemically or as a fusion protein) the αβhIL2 mutein provides extended duration of akin to PEGylation and may be produced as a recombinant fusion protein in E. coli. XTEN polymers suitable for use in conjunction with the hIL2 muteins of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193 Jun. 13, 2016. The XTEN polymer fusion protein may incorporate a protease sensitive cleavage site between the XTEN polypeptide and the hIL2 mutein such as an MMP-2 cleavage site.

In some embodiments, extended in vivo duration of action of the αβhIL2 mutein may be achieved by conjugation to one or more water-soluble polymers. Examples of water soluble polymers useful in the practice of the present invention include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol, polysaccharides, poly-alpha-hydroxy acid, polyvinyl alcohol (PVA), polyphosphazene, polyoxazolines (POZ), poly(N-acryloylmorpholine), or a combination thereof.

In some embodiments, extended in vivo duration of action of the αβhIL2 mutein may be achieved by conjugation to one or more polyethylene glycol molecules (“PEGylation”) of the αβhIL2 mutein.

PEGs suitable for conjugation to the αβhIL2 mutein are generally soluble in water at room temperature and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG useful in the present disclosure is not restricted to any particular range. The PEG component of the PEG-IL2 mutein can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, alternatively about 30,000 to about 40,000 daltons. In one embodiment of the invention, the PEG is a 40kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

PEGylation most frequently occurs at the a-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

The PEG can be bound to an αβhIL2 mutein of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present invention include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g. Sunbright® ME-200AL, NOF, a 20 kDa linear PEG- NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10kDA linear PEG molecules (e.g., Sunbright® GL2-200AL3, NOF), a 20 kDa 2-arm branched PEG-NHS ester the 20 kDA PEG-NHS ester comprising two 10kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20kDA linear PEG molecules (e.g., Sunbright® GL2-400AL3), a 40 kDa 2-arm branched PEG-NHS ester the 40 kDA PEG-NHS ester comprising two 20kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

As previously noted, the PEG may be attached directly to the αβhIL2 mutein or via a linker molecule. Suitable linkers include “flexible linkers” which are generally of sufficient length to permit some movement between the modified polypeptide sequences and the linked components and molecules. The linker molecules are generally about 6-50 atoms long. The linker molecules can also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers include glycine polymers (G)ₙ, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G), glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein.

In one embodiment, the branched 40kD PEG and linker conjugated to the N-terminal prolines of a αβhIL2 mutein has the structure:

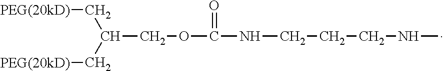

In a particular embodiment of the present disclosure, the αβhIL2 mutein is a hIL2 mutein comprising the amino acid substitutions L18R, Q22E, and Q126K numbered in accordance with SEQ ID NO: 4, a deletion of the N-terminal alanine residue (des-Alal), and comprises a branched 40kD PEG and linker conjugated to the N-terminus of the mutein, the branched 40kD PEG and linker having the structure

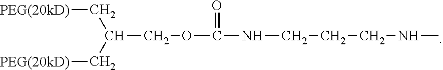

In one embodiment of the disclosure, the αβIL2 mutein modified to provide extended duration of action in vivo is a PEGylated αβIL2 mutein useful in the practice of the present disclosure is of the structure:
[PEG]-[linker]ₙ-[desAla1-hIL2[L18R/Q22E/Q126K]
   wherein n=0 or 1, or

In one embodiment of the disclosure, the αβIL2 mutein modified to provide extended duration of action in vivo is a PEGylated αβIL2 mutein useful in the practice of the present disclosure is of the structure:

40kD-PEG-(linker)n-PTSSSTKKTQLQLEHLRLDLEMILNG
INNYKNPKLTRMLTFKFYMPKKATELKHLQCLEEELKPLEEVLNL
AQSKNFHLRPRDLISNINVIVLELKGSETTFMCEYADETATIVEF
LNRWITFCKSIISTLT

wherein n=0 (absent) or 1 (present).

In one embodiment of the disclosure, the αβIL2 mutein modified to provide extended duration of action in vivo is a PEGylated αβIL2 mutein useful in the practice of the present disclosure is of the structure:

40kD-PEG-(linker)ₙ-PTSSSTKKTQLQLEHLRLDLEMILNG
INNYKNPKLTRMLTFKFYMPKKATELKHLQCLEEELKPLEEVLNL
AQSKNFHLRPRDLISNINVIVLELKGSETTFMCEYADETATIVEF
LNRWITFCKSIISTLT

wherein the 40kD-PEG-Linker (n=1) is a molecule of the structure:

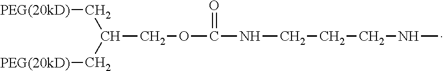

Although the method or site of PEG attachment to the αβhIL2 mutein may vary, in certain embodiments the PEGylation does not alter, or only minimally alters, the activity of the αβhIL2 mutein. Site specific PEGylation of the αβhIL2 mutein may be employed to avoid interference o the PEG with the binding properties of the binding to one or more of the IL2 receptor subunits. Site specific pegylation of the αβhIL2 mutein may be achieved by substitution of one or more amino acids for naturally occurring amino acid the side chain of which facilitates PEGylation (e.g., cysteine) or by site specific the incorporation of non-natural amino acids having side chains to facilitate selective PEG conjugation. For example, the αβhIL2 mutein may comprise a substitution of a cysteine may be for the threonine at position 3 (3TC) to facilitate N-terminal PEGylation using particular chemistries. Incorporation of non-natural amino acids having side chains to facilitate selective PEG conjugation chemistries as described Ptacin, et al., (PCT International Application No. PCT/US2018/045257 filed Aug. 3, 2018, and published Feb. 7, 2019 as International Publication Number WO 2019/028419A1.

Conversely, site specific conjugation of the PEG to an hIL2 mutein to may be used to generate an αβhIL2 mutein by incorporating non-natural amino acids having a PEGylatable specific moiety at those sequences or residues of hIL2 identified as interacting with hCD132 including amino acids such as residues 18, 22, 109, 126, and 119-133. Site-specific PEGylation at one or more of these residues which have been identified at the interface between IL2 and CD132 may be used to prepare an αβhIL2 mutein having diminished binding to hCD132 useful in the practice of the methods of the present disclosure.

In some embodiments an αβhIL2 mutein having an extended duration of action in a mammalian subject and useful in the practice of the present disclosure is achieved by covalent attachment of the αβhIL2 mutein to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the αβhIL2 mutein is acetylated at the N-terminus by enzymatic reaction with N-terminal acetyltransferase and, for example, acetyl CoA. Alternatively, or in addition to N-terminal acetylation, the αβhIL2 mutein is acetylated at one or more lysine residues, e.g., by enzymatic reaction with a lysine acetyltransferase. See, for example Choudhary et al. (2009) Science 325 (5942):834L2 ortho840.

In some embodiments, embodiment, the αβhIL2 mutein may comprise a functional domain of a chimeric polypeptide. hIL2 mutein fusion proteins of the present disclosure may be readily produced by recombinant DNA methodology by techniques known in the art by constructing a recombinant vector comprising a nucleic acid sequence comprising a nucleic acid sequence encoding the αβhIL2 mutein in frame with a nucleic acid sequence encoding the fusion partner either at the N-terminus or C-terminus of the hIL2 mutein, the sequence optionally further comprising a nucleic acid sequence in frame encoding a linker or spacer polypeptide.

In other embodiments, the αβhIL2 mutein can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the hIL2 mutein polypeptide further comprises a C-terminal c-myc epitope tag.

In other embodiments, the αβhIL2 mutein can be modified to include an additional polypeptide sequence that facilitates isolation or purification. Non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

In some embodiment, the αβhIL2 mutein (including an αβhIL2 mutein fusion protein) of the present disclosure are expressed as a fusion protein with one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986.

Examples of transition metal chelating polypeptides useful in the practice of the present invention are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present invention are peptides comprising 3-6 contiguous histidine residues such as a six-histidine peptide (His)₆ and are frequently referred to in the art as “His-tags.”

In some embodiments, the αβhIL2 mutein is conjugated to a molecule (“targeting domain”) which provides selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the αβhIL2 mutein sequence and the sequence of the targeting domain of the fusion protein.

In other embodiments, a chimeric polypeptide including a αβhIL2 mutein and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135. In some embodiments, the targeting moiety is an antibody (including single domain antibodies such as VHHs, scFvs) that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3Ra2, CD19, mesothelin, Her2, EpCam, Mucd, ROR1, CD133, CEA, EGRFRVIII, PSCA, GPC3, Pan-ErbB and FAP.

In other embodiments, the chimeric polypeptide includes the αβhIL2 mutein and a heterologous polypeptide that functions to enhance expression or direct cellular localization of the αβhIL2 mutein, such as the Aga2p agglutinin subunit (see, e.g., Boder and Wittrup, Nature Biotechnol. 15:553-7, 1997).

In some embodiments, the targeting moiety may be an antibody or antibody fragment. In particular, antibodies that are selective for binding to tumor cell associated antigens are useful in the targeted delivery of a systemically administered αβhIL2 mutein to the tumor and provide support for the antigen specific TILs in the tumor.

In some embodiments, the αβhIL2 mutein also may be linked to additional therapeutic agents including therapeutic compounds such as anti-inflammatory compounds or antineoplastic agents, therapeutic antibodies (e.g. Herceptin), immune checkpoint modulators, immune checkpoint inhibitors (e.g. anti-PD1 antibodies), cancer vaccines as described elsewhere in this disclosure. Anti-microbial agents include aminoglycosides including gentamicin, antiviral compounds such as rifampicin, 3′-azido-3′-deoxythymidine (AZT) and acylovir, antifungal agents such as azoles including fluconazole, plyre macrolides such as amphotericin B, and candicidin, anti-parasitic compounds such as antimonials, and the like. The hIL2 mutein may be conjugated to additional cytokines as CSF, GSF, GMCSF, TNF, erythropoietin, immunomodulators or cytokines such as the interferons or interleukins, a neuropeptide, reproductive hormones such as HGH, FSH, or LH, thyroid hormone, neurotransmitters such as acetylcholine, hormone receptors such as the estrogen receptor. Also included are non-steroidal anti-inflammatories such as indomethacin, salicylic acid acetate, ibuprofen, sulindac, piroxicam, and naproxen, and anesthetics or analgesics. Also included are radioisotopes such as those useful for imaging as well as for therapy.

The αβhIL2 muteins of the present disclosure may be chemically conjugated to such carrier molecules using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to αβhIL2 mutein and can readily be identified by those skilled in the art. Alternatively, or in addition, the αβhIL2 mutein and/or the molecule to which it is intended to be conjugated may be chemically derivatized such that the two can be conjugated in a separate reaction as is also well known in the art.

In general, in the practice of adoptive cell therapy, during the ex vivo phase, a sample of a tissue (e.g., a neoplasm) comprising T cells (e.g., TILS) is obtained and subjected to a two-step process: an initial outgrowth step and a rapid expansion (REP) step.

The out-growth step begins with the excision of a sample of a neoplasm which is cut into small pieces (of a few millimeters) or enzymatically digested into a single cell suspension. Fragments or digests are then cultured in the presence of an αβhIL2 at a concentration sufficient to induce proliferation (e.g., at or above EC₁₀^{PRO} of the alternatively at or above EC₂₀^{PRO}, alternatively at or above EC₃₀^{PRO}, alternatively at or above EC₄₀^{PRO}, at or above EC₅₀^{PRO}, alternatively at or above EC₆₀^{PRO} of the αβhIL2 mutein) for a period of from about 7 to 21 days, alternatively from about 12-18 days, alternatively from about 12-16 days, about 12, days, about 13 days, about 14 days. The culture is maintained until there are approximately 5×10⁷ TILs. During the outgrowth of a digest, tumor cells typically disappear from the cultures. The use of tumor fragments or digest during the outgrowth phase does not typically to influence the success rates of outgrowth and/or clinical response.

In some embodiments, the outgrowth step may optionally provide by a selection step to further enrich the population of cells for tumor specific. Once culture consisted mostly of CD3+ T cells, their specificity is tested during a short culture in the presence of an autologous or HLA-matched tumor cell line by quantification of interferon-y (IFN-γ). The selected cell populations are then further expanded as described above for an additional period of time in substantial accordance with procedure described immediately above.

During the ex vivo phase, the activated tumor reactive T cells may be further selected and sorted and enriched (e.g., by FACS) based one or more additional cell surface markers. Improvement in TIL therapy is reported with selecting for cells which express PD1, consequently, in some embodiments, the T cell possesses CD8 and CD25 and PD1 (i.e., CD8+ CD25+PD1+ T cells). Enrichment/selection for CD8 positive PD1 Positive T cells: In some embodiments, the pre-selection procedure involves the enrichment of the cell population of PD-1+ CD8+ T cells. Salas-Benito, et al D(2018) J Immunol Sci. (2018); 2(1): 55-59 report that pre-selection of PD-1+ tumor-infiltrating CD8+ T cells prior to ex vivo expansion improves the efficacy of TIL adoptive T-cell therapy. PD-1+ CD8 T cells can be easily and rapidly isolated using FACS or magnetic technologies. The use of pre-enriched tumor-specific T cells may simplify the TIL production method and, at the same time, may help to generate T-cell products with high antitumor activity. PD-1 may enable the isolation of rare tumor-specific TILs and allow TIL therapy to be facilitate the application of TIL therapy to solid tumors.

In the REP step, the cells obtained from the outgrowth step are stimulated and further expanded to large numbers (typically between 1×10¹⁰ and 2×10¹¹ cells). The cells obtained from the outgrowth step are mixed with a 100-200 fold excess of irradiated feeder cells (from autologous or allogenic source) in the presence of biased IL2 mutein of the present disclosure at a concentration sufficient to induce proliferation (e.g., at or above EC₁₀^{PRO}, alternatively at or above EC₂₀^{PRO}, alternatively at or above EC₃₀^{PRO}, alternatively at or above EC₄₀^{PRO}, at or above EC₅₀^{PRO}, alternatively at or above EC₆₀^{PRO}) for a period of from about 7 to 21 days, alternatively from about 12-18 days, alternatively from about 12-16 days, about 12, days, about 13 days, about 14 days. The irradiated feeder cells release growth factors into the culture which will accommodate massive TIL expansion, usually more than 1000-fold. During the last phase of the REP, a bioreactor (such as WAVE or Xuri, or gas permeable G Rex bottles) is typically employed to facilitate culture of high cell densities. The REP step may optionally be performed in presence of an activating compound such as a CD3 antibody.

In some embodiments, the TIL may optionally be engineered during the ex vivo phase using technologies well known in the art such as CRISPR-cas9 or expression vectors (e.g., lentiviral expression vectors or mRNA) to express additional proteins that aid in anti-tumor effect (e.g CXCR2 receptor) as described in Forget, et al (2017) Frontiers in Immunology 8:908 and Idorn, et al (2016) Methods Mol Biol. 1428:261-76.

In the in vivo phase of adoptive T cell therapy, the expanded T cells obtained from the ex vivo phase are re-administered to the subject in the presence of a biased IL2 mutein of the present disclosure at concentration sufficient to expand the activated cell population, optionally in combination with one or more supplementary agents.

In some embodiments, the αβhIL2 muteins useful in the practice in vivo phase of the methods of the present disclosure provide modifications that modify the binding of the IL2 mutein to other proteins, in particular CD25, CD122 and CD132 as well as combinations of such proteins such as CD122/CD132 (the “intermediate affinity IL2 receptor”), CD25 (the “low affinity IL2 receptor”) and CD25/CD122/CD132 (the “high affinity IL2 receptor”). The present disclosure provides methods and compositions for the treatment and/or prevention of neoplastic diseases, disorders or conditions by the administration of a therapeutically effective amount of an human IL-2 muteins that have decreased binding affinity for CD132 yet retain significant binding affinity for CD122 and/or CD25 comparable to the affinity of wild-type human IL-2.

In some embodiments, the IL2 muteins useful in the practice of the methods of the present disclosure possess decreased binding affinity to the extracellular domain of hCD132 (e.g, <50% the affinity of wild type hIL2, alternatively <45% the affinity of wild type hIL2, alternatively <40% the affinity of wild type IL2, alternatively <35% the affinity of wild type hIL2, alternatively <25% the affinity of wild type hIL2, alternatively <20% the affinity of wild type hIL2, alternatively <15% the affinity of wild type IL2, alternatively <10% the affinity of wild type IL2, or alternatively <5% the affinity of wild type IL2) while retaining substantial affinity (e.g., 20% the affinity of wild type hIL2, alternatively >30% the affinity of wild type hIL2, alternatively >40%, alternatively >50% the affinity of wild type hIL2, alternatively >60% the affinity of wild type hIL2, alternatively >65% the affinity of wild type hIL2, alternatively >70% the affinity of wild type hIL2, alternatively >75% the affinity of wild type hIL2, alternatively >80% the affinity of wild type hIL2, alternatively >85% the affinity of wild type hIL2, alternatively >90% the affinity of wild type IL2, alternatively >90% the affinity of wild type IL2, alternatively >95% the affinity of wild type IL2, alternatively >100% the affinity of wild type IL2, alternatively >105% the affinity of wild type hIL2, alternatively >110% the affinity of wild type IL2, alternatively >115% the affinity of wild type hIL2, alternatively >125% the affinity of wild type IL2, or alternatively >150% the affinity of wild type hIL2) binding affinity for the extracellular domain of the wild type human CD122 receptor.

In some embodiments, the αβhIL2 mutein useful in the practice of the methods of the present disclosure has reduced binding affinity for the extracellular domain of hCD132 receptor further includes 1, 2, 3, 4, 5, 6, 7, 8, 9, or 10 or more mutations that increase affinity for the extracellular domain of the wild type human CD122 receptor. In certain embodiments, the αβhIL2 mutein useful in the practice of the methods of the present disclosure includes at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) relative to a wild type IL-2 (e.g., SEQ ID NO: 4), and binds the CD122 with higher affinity than a wild type IL-2. In certain embodiments, the αβhIL2 mutein binds CD122 with an affinity that is at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% greater than wild type IL-2. The binding affinity of αβhIL2 mutein can also be expressed as 1.2, 1.4, 1.5, 2, 5, 10, 15, 20, 25, 50, 100, 200, 250 or more fold greater affinity for the extracellular domain of hCD122 than wild type hIL-2.

In some embodiments, the αβhIL2 muteins useful in the practice of the methods of the present disclosure possess decreased binding affinity to the extracellular domain of hCD132 (e.g, <50% the affinity of wild type hIL2, alternatively <45% the affinity of wild type hIL2, alternatively <40% the affinity of wild type hIL2, alternatively <35% the affinity of wild type hIL2, alternatively <25% the affinity of wild type hIL2, alternatively <20% the affinity of wild type hIL2, alternatively <15% the affinity of wild type hIL2, alternatively <10% the affinity of wild type hIL2, or alternatively <5% the affinity of wild type hIL2) while retaining substantial affinity (e.g, >50% the affinity of wild type hIL2, alternatively >60% the affinity of wild type hIL2, alternatively >65% the affinity of wild type hIL2, alternatively >70% the affinity of wild type hIL2, alternatively >75% the affinity of wild type hIL2, alternatively >80% the affinity of wild type hIL2, alternatively >85% the affinity of wild type hIL2, alternatively >90% the affinity of wild type hIL2, alternatively >90% the affinity of wild type hIL2, alternatively >95% the affinity of wild type hIL2, alternatively >100% the affinity of wild type hIL2, alternatively >105% the affinity of wild type hIL2, alternatively >110% the affinity of wild type hIL2, alternatively >115% the affinity of wild type hIL2, alternatively >125% the affinity of wild type hIL2, or alternatively >150% the affinity of wild type IL2) in the hCD25/hCD122 receptor complex. In certain embodiments, the αβhIL2 muteins of the present disclosure possess reduced affinity for CD132. In some embodiments, such IL2 muteins incorporate modifications to the primary structure of the wild type IL2 incorporating one or more modifications at positions 18, 22, and 126 numbered in accordance with wild type hIL-2.

In some embodiments, the αβhIL2 muteins useful in the practice of the methods of the present disclosure possess decreased binding affinity to CD132 while retaining substantial affinity (e.g. >50% the affinity of wild type hIL2, alternatively >60% the affinity of wild type hIL2, alternatively >65% the affinity of wild type hIL2, alternatively >70% the affinity of wild type hIL2, alternatively >75% the affinity of wild type hIL2, alternatively >80% the affinity of wild type hIL2, alternatively >85% the affinity of wild type hIL2, alternatively >90% the affinity of wild type hIL2, alternatively >90% the affinity of wild type IL2, alternatively >95% the affinity of wild type hIL2, alternatively >100% the affinity of wild type IL2, alternatively >105% the affinity of wild type hIL2, alternatively >110% the affinity of wild type hIL2, alternatively >115% the affinity of wild type hIL2, alternatively >125% the affinity of wild type hIL2, alternatively >150% the affinity of wild type hIL2, alternatively >200% the affinity of wild type hIL2, alternatively >300% the affinity of wild type IL2, alternatively >400% the affinity of wild type hIL2, alternatively >500% the affinity of wild type IL2) binding affinity for hCD25.

In some embodiments, the IL2 muteins useful in the practice of the methods of the present disclosure exhibit significant or enhanced binding affinity for hCD25 and reduced binding affinity for the extracellular domain of hCD132 receptor as compared to wild type human IL-2 (hIL-2).

In some embodiments, the αβhIL2 muteins useful in the practice of the methods of the present disclosure comprise one or more amino acid substitutions that decrease CD132 receptor binding affinity selected from amino acid positions 18, 22, and 126, numbered in accordance with mature wild type hIL-2.

In some embodiments, the αβhIL2 muteins useful in the practice of the methods of the present disclosure that are partial agonists have one or more reduced functions as compared to wild type IL-2.

In certain embodiments, the αβhIL2 muteins useful in the practice of the methods of the present disclosure disrupt the association of the CD122 with the CD132 such that this CD122/CD132 interaction is reduced by about 2%, about 5%, about 10%, about 15%, about 20%, about 50%, about 75%, about 90%, about 95% or more relative to wild type hIL-2. In some embodiments, the one or more mutations reducing the binding affinity of the IL-2 mutein for CD132 is an amino acid substitution. In some embodiments, the subject hIL-2 mutein consists of 1, 2, 3, 4, 5, 6, 7, 8, 9, 10, 11, 12, 13, 14, or 15 amino acid substitutions as compared to a wild type IL-2 (SEQ ID NO: 4).

In some embodiments, the αβhIL2 muteins useful in the practice of the methods of the present disclosure are partial agonists. In certain embodiments, the αβhIL2 muteins useful in the practice of the methods of the present disclosure is a partial agonist has reduced capabilities to stimulate one or more signaling pathways that are dependent on CD122/CD132 heterodimerization. In some embodiments, the αβhIL2 muteins has a reduced capability to stimulate phosphorylation in an CD122+ cell as compared to wild type hIL-2. In some embodiments, the IL-2 mutein stimulates STAT5 phosphorylation in an IL-2RP+ cell at a level that is 1%, 5%, 1%, 15%, 20%, 25%, 30%, 35%, 40, 45%, 50%, 55%, 60%, 65%, 70%, 75%, 80%, 85%, 90%, 95% or less of the level that wild type IL-2 stimulates STAT5 phosphorylation in the same cell.

In some embodiments, the αβhIL2 muteins useful in the practice of the methods of the present disclosure are partial agonists having a reduced capability to stimulate signaling in an CD122+ cell as compared to wild type hIL-2. In some embodiments, the αβhIL2 mutein stimulates pERK1/ERK2 signaling in an CD122+ cell at a level that is 1%, 5%, 10%, 15%, 20%, 25%, 30%, 35%, 40%, 45%, 50%, 55%, 60%, 65%, 70%, 75%, 80%, 85%, 90%, 95% or less of the level that wild type IL-2 stimulates pERK1/ERK2 signaling in the same cell. In some embodiments, the CD122+ cell is a T cell. In particular embodiments, the CD122+ T cell is a CD8+ T cell. In some embodiments, the CD122+ CD8+ T cell is a CD122+ CD8+ T cell isolated from a subject. In other embodiments, the CD8+ T cell is an activated CD122+ CD8+ T cell. In other embodiments, the CD122+ cell is a natural killer (NK) cell. STAT5 and ERK1/2 signaling can be measured, for example, by phosphorylation of STAT5 and ERK1/2 using any suitable method known in the art. For example, STAT5 and ERK1/2 phosphorylation can be measured using antibodies specific for the phosphorylated version of these molecules in T cells.

In certain embodiments, the αβhIL2 muteins useful in the practice of the methods of the present disclosure are partial agonists having has a reduced capability to induce lymphocyte proliferation as compared to wild type hIL-2. In some embodiments, the lymphocyte is a T cell. In particular embodiments, the lymphocyte is a primary CD8+ T cell. In other embodiments, the lymphocyte is an activated CD8+ T cell. Cell proliferation can be measured using any suitable method known in the art. For example, lymphocyte proliferation can be measured using a carboxyfluorescein diacetate succinimidyl diester (CFSE) dilution assay or by thymidine incorporation. In some embodiments, an αβhIL2 mutein of the present disclosure induces lymphocyte proliferation at a level that is 1%, 5%, 10%, 15%, 20%, 25%, 30%, 35%, 40%, 45%, 50%, 55%, 60%, 65%, 70%, 75%, 80%, 85%, 90%, 95% or less of the level that wild type hIL-2 induce lymphocyte proliferation.

In some embodiments, the αβhIL2 muteins useful in the practice of the methods of the present disclosure are partial agonists that has a reduced capability to activate CD25 expression in a lymphocyte as compared to wild type IL-2. In some embodiments, the αβhIL2 mutein activates CD25 expression in a lymphocyte at a level that is 1%, 5%, 10%, 15%, 20%, 25%, 30%, 35%, 40%, 45%, 50%, 55%, 60%, 65%, 70%, 75%, 80%, 85%, 90%, 95% or less of the level that wild type IL-2 activates CD25 expression in the same cell. In some embodiments, the lymphocyte is a CD8+ T cell. In some embodiments, the CD8+ T-cell is a freshly isolated CD8+ T cell. In other embodiments, the CD8+ T cell is an activated CD8+ T cell.

In some embodiments, the αβhIL2 muteins useful in the practice of the methods of the present disclosure are full agonists.

In some embodiments, the αβhIL2 muteins useful in the practice of the methods of the present disclosure are super agonists.

In some embodiments, the disclosure provides methods and compositions for the treatment and/or prevention of neoplastic diseases, disorders or conditions by the administration of a population of CD8+ CD25+ enriched T cells in combination with a therapeutically effective amount of an αβhIL2 mutein optionally in combination with one or more supplementary agents, including but not limited to one or more of chemotherapeutics, immune checkpoint modulators, radiotherapy and/or physical interventional treatment methods such as surgery.

In some embodiments the present disclosure provides methods and compositions for the treatment and/or prevention of neoplastic diseases, disorders or conditions by the administration of a population of CD8+ CD25+ enriched T cells in combination with a therapeutically effective amount of an αβhIL2 mutein that has decreased binding affinity for CD132 yet retain significant binding affinity for CD122 and/or CD25 comparable to the activity of wild-type human IL-2 wherein the serum concentration of the αβhIL2 mutein is maintained for a majority (i.e., greater than about 50% of the period of time, alternatively greater than about 60%, alternatively greater than about 70%, alternatively greater than about 80%, alternatively greater than about 90%) of a period of time (e.g. at least 24 hours, alternatively at least 48 hours, alternatively at least 72 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer) at a serum concentration at or above the effective concentration of the IL2 mutein sufficient to promote proliferation of CD3-activated primary human T-cells (e.g., at or above EC₁₀^{PRO}, alternatively at or above EC₂₀^{PRO}, alternatively at or above EC₃₀^{PRO} alternatively at or above EC₄₀^{PRO}, at or above EC₅₀^{PRO}, alternatively at or above EC₆₀^{PRO}) with respect to αβhIL2 mutein and at a serum concentration at or above of the effective concentration at a serum concentration of such IL2 mutein sufficient to induce activation of T-cells (e.g., at or above the EC₇₀^{ACT}, alternatively at or above the EC₆₀^{ACT}, alternatively at or above the EC₅₀^{ACT}, alternatively at or above the EC₅₀^{ACT}, at at or above the EC₄₀^{ACT}alternatively at or above the EC₄₀^{ACT}) with respect to such αβhIL2 mutein.

In some embodiments, the present disclosure provides methods of use of a population of CD8+ CD25+ enriched T cells in combination with one or more αβhIL2 muteins for the treatment of neoplastic disease.

During the in vivo phase, the αβhIL2 mutein may be administered in combination with one or more supplementary agents as described below. In some embodiments, administration of the αβhIL2 mutein to the subject occurs in advance of the administration of the enriched population of adoptive T cells.

In some embodiments of the methods of the present disclosure, the subject is optionally subjected to lymphodepleting non-myeloablative chemotherapeutic regimen (NMA chemotherapy) prior to the in vivo phase and readministration of the expanded cell population. Multiple studies have been performed evaluating the role of preconditioning lymphodepleting regimens. Lymphodepleting regimens cause a short, but deep lymphopenia and neutropenia, with full bone marrow recovery within 7-10 days, not requiring hematopoietic stem cell support. In one embodiment the NMA comprises the following regimen: approximately of 2 days intravenous administration of cyclophosphamide at a dose of approximately 60 mg/kg followed by 5 days fludarabine at a dose of approximately 25 mg/m².

In some embodiments, the lymphodepleting regimen comprises the administration of cyclophosphamide and fludarabine. Lymphodepletion regimens are commonly employed in combination with adoptive cell therapy protocols and the agents and dose ranges for the administration of lymphodepleting agents are well known in the art. In one embodiment of the practice of the foregoing method, the subject is treated with a lymphodepletion regimen comprising cyclophosphamide in combination with fludarabine. In some embodiments the lymphodepletion regimen involves the administration cyclophosphamide in combination with fludarabine for a period of 1, 2, 3, 4, or 5 days prior to the administration of the adoptively transferred cells. In some embodiments the dose of cyclophosphamide used in the lymphodepletion regimen is from about 100, 200, 300, 400, 500, 600 mg/m²/day over the course of 1, 2, 3, 4, or 5 days prior to the administration of adoptively transferred cells cells. In some embodiment, the lymphodepleting regimen comprises the administration of the subject of cyclophosphamide 300 mg/m²/day and fludarabine 30 mg/m²/day for a period of three days. In some embodiments the dose of fludarabine used in the lymphodepletion regimen is from about 10, 20, 30, 40, 50, 60 mg/m²/day over the course of 1, 2, 3, 4, or 5 days prior to the administration of the GPC CAR T cells. In one embodiment, the dose of cyclophosphamide at about 500 mg/m²/day to about 600 mg/m²/day and a dose of fludarabine at about 30 mg/m²/day for a period of three days prior to administration of the adoptively transferred cells. In one embodiment, the dose of cyclophosphamide at about 300 mg/m²/day to about 600 mg/m²/day and a dose of fludarabine at about 30 mg/m²/day for a period of three days prior to administration of the adoptively transferred cells.

In some embodiments of the methods of the present disclosure, the subject is optionally or additionally lymphodepleted with total body ionizing irradiation (TBI) at a dose of from about 1 gray to about 80 gray, optionally from about 1 gray to about 20 gray, optionally from about 2 gray to about 15 gray. Murine models had shown that response rates upon TIL therapy improved after prior lymphodepletion by total body irradiation (TBI). These models showed that depletion of endogenous lymphocytes created physical space, resulted in less competition for homeostatic cytokines IL-7 and IL-15 and removed immunosuppressive lymphoid and myeloid populations.

In radiation therapy, the amount of radiation applied varies depending on the type and stage of cancer being treated. Higher doses of radiation are typically administered in the case of solid epithelial tumors where lower doses may be sufficient for non-solid tumors such as lymphomas, and as part of a maintenance protocol from about 0.5gray to about 4 gray, preferably about 1-2 gray.

In an alternative embodiments to the administering the abhIL2 mutein to a subject to provide in vivo support for tumor antigen specific activated T cells prepared in accordance the methods of the present disclosure, the enriched cell population comprising the tumor antigen experienced activated T cells may be selectively activated through the use of a engineered receptor-ligand pair that provides for selective proliferation and activation of the cells expressing the engineered receptor in a subject in response to the administration of the the cognate ligand for the engineered receptor.

In some embodiments, in response to binding of the cognate ligand to the extracellular domain (ECD) of the engineered receptor, the intracellular domain (ICD) of the engineered receptor initiates intracellular signaling in the TIL results in activation and/or proliferation of the engineered cell. In some embodiments the engineered receptor comprises ICD which of which activates the JAK/STAT pathway in a T cell, such that contacting a T cell expressing the engineered receptor with its cognate ligand results in the JAK/STAT signaling in the cell resulting in activation and/or proliferation of the T cell expressing the engineered receptor.

The disclosure further provides a method of treating a subject suffering from a neoplastic disease, said method comprising the steps of:
a. isolating a tissue sample from the subject suffering from a neoplastic disease, the tissue sample comprising a population of TILs;
b. contacting the tissue sample of step (a) ex vivo with a quantity of an αβhIL2 mutein at a concentration sufficient to induce proliferation and activation of the TILs to generate an expanded cell population comprising activated TILs; and
c. contacting the expanded cell population of step (b) an expression vector, the expression vector comprising a nucleic acid sequence encoding an engineered receptor operably linked to one or more expression control sequences active in a T cell, receptor,
d. administering the cell population comprising activated TILs recombinantly modified to express the engineered receptor prepared in accordance with step (c) to the subject,
e. administering to the subject a quantity of a cognate ligand that specifically binds to the extracellular domain of the engineered receptor wherein the binding of the ligand to the receptor results in intracellular signaling in the TILs expressing the receptor, wherein such intracellular signaling results in the activation and proliferation of the TIL expressing the engineered receptor.

As the antigen experienced TILs expanded in response the administration of the αβhIL2 mutein do not bind to the same antigen on the tumor cell but rather are polyclonal by their nature, the foregoing methods provide methods of providing a polyclonal antitumor response in the subject that may be modulated in response to the administration of the cognate ligand for the engineered receptor. Consequently, the foregoing method provides a method of inducing a polyclonal antitumor immune response in a subject, the polyclonal response capable of modulation in response to the administration to the subject of an effective amount of a ligand that specifically binds to and activates intracellular signaling in the engineered cells expressing engineered receptor.

A variety of engineered receptor ligand pairs that result in activation and/or proliferation of T cells expressing the engineered receptor in response to contact by a cognate ligand are known in the art and may be employed in the method of the present disclosure.

In one embodiment, the engineered receptor/ligand pair is the “orthogonal” IL2 receptor ligand system described in Garcia, et al. U.S. Pat. No. 10,869,887 issued Dec. 22, 2020, the entire teaching of which is incorporated by reference. Garcia et al describes a hCD122 receptor subunit that has been modified at positions 133 and/or 134 of the ECD of the hCD122. These modifications effectively abolish binding of wild-type hIL2 to the engineered receptor. However, Garcia, et al further engineered hIL2 variants that selectively binding to the ECD of the modified hCD122 receptor such that the engineered hIL2 variant is capable of selectively activating the JAK/STAT signaling cascade of the ICD of the hCD122 resulting in selective activation and/or proliferation of T cells expressing the modified CD122 receptor in vivo in response to administration of the engineered hIL2 variant ligand to the subject. The engineering of TILs to express the receptors described in Garcia, et al. is described in Chartier-Courtaud, et al., PCT International Application Number PCT/US20/065892 published as WO 2020/131547on Jun. 25, 2020.

In one embodiment, the present disclosure provides a method of treating a subject suffering from a neoplastic disease, said method comprising the steps of:
a. isolating a tissue sample from the subject suffering from a neoplastic disease, the tissue sample comprising a population of TILs;
b. contacting the tissue sample of step (a) ex vivo with a quantity of an αβhIL2 mutein at a concentration sufficient to induce proliferation and activation of the TILs to generate an expanded cell population comprising activated TILs; and
c. contacting the expanded cell population of step (b) an expression vector, the expression vector comprising a nucleic acid sequence encoding an engineered operably linked to one or more expression control sequences active in a T cell, receptor,
d. administering the cell population comprising activated TILs recombinantly modified to express the engineered receptor in accordance with step (c) to the subject, wherein the receptor is a hCD122 comprising the amino acid substitutions at positions H133 and Y134
e. administering to the subject a quantity of a cognate ligand that specifically binds to the extracellular domain of the engineered receptor wherein the binding of the ligand to the receptor results in intracellular signaling in the TILs expressing the receptor, wherein the cognate ligand is a hIL2 mutein comprising wherein such intracellular signaling results in the activation and proliferation of the TIL expressing the receptor.

In some embodiments, the engineered receptor is a human CD122 protein comprising amino acid substitutions H133D and Y134F. In some embodiments, the expression vector is a lentiviral vector or a retroviral vector. In one embodiment the engineered receptor is a human CD122 protein comprising amino substitutions at positions 133 and 134 and the engineered ligand is the as described in Garcia et al and the engineered ligand is a human IL2 mutein comprising an amino acid substitution at position 15 selected from E15S, E15T, E15Q, or E15H; an amino acid substitution at position 16 of H16Q; an amino acid substitution at position 19 selected from L19V or L19I; an amino acid substitution at position 20 selected from D20T, D20S, D20L or D20M; and an amino acid substitution at position 23 selected from M23L, M23S, M23V, M23A, or M23T; and optionally further comprises an amino acid substitution at position 22 selected from Q22K, Q22N, R81D, R81Y, T51I or a combination thereof. In some embodiments, the engineered ligand is a human IL2 mutein comprising the substitutions E15S, H16Q, L19V, D20L; Q22K and M23A (referred to as SQVLKA; SEQ ID NO: 6). In some embodiments the engineered ligand is modified to provide an extended half-life in vivo as more fully described elsewhere herein. In one embodiment, the engineered ligand is a PEGylated version of SQVLKA comprising a des-Ala1 deletion (SEQ ID NO:7) and the addition of an N-terminal 40 kDa branched PEG to P2 of the des-Ala1 SQVLKA ligand.

In one embodiment of the disclosure, the cognate ligand is a human IL2 variant of the structure:
[PEG]-[linker]ₙ-[desAla1-hIL2[E15S-H16Q-L19V-D20L-Q22K-M23A]
   wherein n=0 or 1, or
In another embodiment of the disclosure, the cognate ligand is a human IL2 variant of the structure

(SEQ ID NO: 7)
40kD-PEG-(linker)ₙ-PTSSSTKKTQLQLSQLLVLLKAILNG
INNYKNPKLTRMLTFKFYMPKKATELKHLQCLEEELKPLEEVLNL
AQSKNFHLRPRDLISNINVIVLELKGSETTFMCEYADETATIVEF
LNRWITFCQSIISTLT

wherein n=0 (absent) or 1 (present).

In one embodiment, the cognate ligand is a human IL2 variant of the structure

(SEQ ID NO: 7)
40kD-PEG-(linker)ₙ-PTSSSTKKTQLQLSQLLVLLKAILNG
INNYKNPKLTRMLTFKFYMPKKATELKHLQCLEEELKPLEEVLNL
AQSKNFHLRPRDLISNINVIVLELKGSETTFMCEYADETATIVEF
LNRWITFCQSIISTLT

wherein the 40kD-PEG-Linker (n=1) is a molecule of the structure:

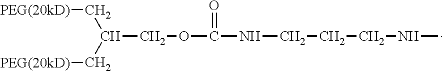

In some embodiments, the present disclosure provides for the administration of a pharmaceutical formulation comprising a therapeutically effective amount of cognate ligand to a subject in need of treatment. Administration to the subject may be achieved by intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The cognate ligand may also be suitably administered by intratumoral, peritumoral, intralesional, intranodal or perilesional routes or to the lymph, to exert local as well as systemic therapeutic effects.

A therapeutically effective amount N-terminal 40 kDa branched PEG-des-Alal SQVLKA ligand is from about 0.5 mg to about 20 mg, alternatively from about 1 mg to about 15 mg, alternatively from about 1.5 mg to about 12 mg administered subcutaneously weekly. In one embodiment, a therapeutically effective amount of an N-terminal 40 kDa branched PEG-des-Ala1 SQVLKA ligand for a human subject is from about 1.5 mg to about 12 mg administered subcutaneously weekly.

An alternative ligand/receptor system useful in the practice of the foregoing method is describe in Price et al. United States Patent Application Publication NO US2021/0205365A1 published Jul. 8, 2021. Price et al describe a chimeric growth factor a chimeric growth factor receptor that is selectively activatable in T cells (e.g., NK cells, CARs, TILs) in response to the administration of the approved small molecule thrombopoietin receptor agonist, eltrombopag (commercially available as Promacta®, Novartis). In some embodiments of the foregoing method, the engineered receptor is a chimeric receptor of Price, et al and the activating ligand is eltrombopag.

To provide expression of the engineered receptor in a T cell, the nucleic acid sequence encoding the engineered receptor is incorporated into a vector comprising, the nucleic acid sequence operably linked to one or more expression control sequences functional in the T cell. Viral vector systems useful in the practice of the instant invention include, for example, naturally occurring or recombinant viral vector systems. Viral vectors can be derived from the genome of human or bovine adenoviruses, vaccinia virus, lentivirus, herpes virus, adeno-associated virus, human immunodeficiency virus, sindbis virus, and retroviruses (including but not limited to Rous sarcoma virus), and hepatitis B virus. Typically, genes of interest are inserted into such vectors to allow packaging of the gene construct, typically with accompanying viral genomic sequences, followed by infection of a sensitive host cell resulting in expression of the gene of interest (e.g the engineered receptor). When a viral vector system is to be employed for transfection, retroviral or lentiviral expression vectors are preferred to transfect T-cells due to an enhanced efficacy of gene transfer to T-cells using these systems resulting in a decreased time for culture of significant quantities of T-cells for clinical applications. In particular, gamma retroviruses a particularly preferred for the genetic modification of clinical grade T-cells and have been shown to have therapeutic effect. Pule, et al. (2008) Nature Medicine 14(11):1264-1270. Similarly, self-inactivating lentiviral vectors are also useful as they have been demonstrated to integrate into quiescent T-cells. June, et al. (2009) Nat Rev Immunol 9(10):704-716. Methods of transducing TILs with vectors encoding the modified CD122 protein of Garcia et al are described in Karyampudi, et al., United States Patent Application No. US 2020/0347350A1 published Nov. 5, 2020.

In an alternative to the expression of the orthogonal receptor from a vector, the genome of the cell may be modified to express the orthogonal receptor using techniques known in the art. In some embodiments, the compositions and methods of the present disclosure comprise the step of genetically modifying a human immune cell by using at least one endonuclease to facilitate incorporate the modifications of to the ECD of the engineered hCD122 into the genomic sequence of the human immune cell. Methods for such modification of T cells is described in Galetto, et al. United States Patent Application Publication No. US 2013/015884A1 published Nov. 28, 2013, and methods for TCRalpha deficient T-cells by expressing pTalpha resulting in restoration of a functional CD3 complex as described in Galetto, et al. U.S. Pat. No. 10,426,795B2 issued Oct. 21, 2019.

The IL2 muteins of the present disclosure may be produced by conventional methodology for the construction of polypeptides including recombinant or solid phase syntheses.

αβhIL2 muteins may be generated by affinity maturation of the wild-type hIL2 peptide to enhance affinity for CD25 and/or CD122 and reduced binding affinity of CD132. An “affinity matured” polypeptide is one having one or more alteration(s) in one or more residues which results in an improvement in the polypeptide for a given receptor component relative to the parent wild-type polypeptide. Affinity maturation can be done to increase the binding affinity of the hIL2 mutein by at least about 10%, alternatively at least about 50%, alternatively at least about 100% alternatively at least about 150%, or from 1 to 5 fold as compared to the “parent” polypeptide. The techniques of affinity maturation of polypeptides are well known in the art. See, e.g., Rao, et al (2003) Protein Engineering vol. 16(12): 1081-1087; Levin and Weiss (2006) Molecular BioSystems 2: 49-57. Rao, et al. applied affinity maturation technology of an hIL2 analog having enhanced affinity for CD25.

In addition to generating mutant polypeptides via expression of nucleic acid molecules that have been altered by recombinant molecular biological techniques, subject αβhIL2 muteins can be chemically synthesized. Chemically synthesized polypeptides are routinely generated by those of skill in the art. Chemical synthesis includes direct synthesis of a peptide by chemical means of the protein sequence encoding for an αβhIL2 mutein exhibiting the properties described. This method can incorporate both natural and unnatural amino acids at positions that affect the interactions of IL2 with CD25, CD122 and, CD132.

In some embodiments, the IL2 muteins of the present disclosure may be prepared by chemical synthesis. The chemical synthesis of the IL2 muteins may proceed via liquid-phase or solid-phase. Solid-phase peptide synthesis (SPPS) allows the incorporation of unnatural amino acids and/or peptide/protein backbone modification. Various forms of SPPS are available for synthesizing the IL2 muteins of the present disclosure are known in the art (e.g., Ganesan A. (2006) Mini Rev. Med. Chem. 6:3-10; and Camarero J. A. et al., (2005) Protein Pept Lett. 12:723-8). In the course of chemical synthesis, the alpha functions and any reactive side chains may be protected with acid-labile or base-labile groups that are stable under the conditions for linking amide bonds but can readily be cleaved without impairing the peptide chain that has formed.

In the solid phase synthesis, either the N-terminal or C-terminal amino acid may be coupled to a suitable support material. Suitable support materials are those which are inert towards the reagents and reaction conditions for the stepwise condensation and cleavage reactions of the synthesis process and which do not dissolve in the reaction media being used. Examples of commercially available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. The successive coupling of the protected amino acids can be carried out according to conventional methods in peptide synthesis, typically in an automated peptide synthesizer.

At the end of the solid phase synthesis, the peptide is cleaved from the support material while simultaneously cleaving the side chain protecting groups. The peptide obtained can be purified by various chromatographic methods including but not limited to hydrophobic adsorption chromatography, ion exchange chromatography, distribution chromatography, high pressure liquid chromatography (HPLC) and reversed-phase HPLC.

### Recombinant Production:

Alternatively, the IL2 muteins of the present disclosure are produced by recombinant DNA technology. In the typical practice of recombinant production of polypeptides, a nucleic acid sequence encoding the desired polypeptide is incorporated into an expression vector suitable for the host cell in which expression will be accomplish, the nucleic acid sequence being operably linked to one or more expression control sequences encoding by the vector and functional in the target host cell. The recombinant protein may be recovered through disruption of the host cell or from the cell medium if a secretion leader sequence (signal peptide) is incorporated into the polypeptide. The recombinant protein may be purified and concentrated for further use including incorporation. The process for the recombinant production of IL2 polypeptides is known in the art and described in Fernandes and Taforo, U.S. Pat. No. 4,604,377 issued Aug. 5, 1986, and in Mark, et al., U.S. Pat. No. 4,512,584 issued May 21, 1985, Gillis, U.S. Pat. No. 4,401,756 issued Aug. 30, 1983, the entire teachings of which are herein incorporated by reference.

### Construction of Nucleic Acid Sequences Encoding the IL2 Mutein

In some embodiments, the IL2 mutein is produced by recombinant methods using a nucleic acid sequence encoding the IL2 mutein (or fusion protein comprising the IL2 mutein). The nucleic acid sequence encoding the desired αβhIL2 mutein can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the IL2 mutein (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IL2 mutein may be obtained from various commercial sources that provide custom made nucleic acid sequences. Amino acid sequence variants of the IL2 polypeptides to the produce the IL2 muteins of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding the αβhIL2 muteins and expressing those sequences in a suitably transformed host include, but are not limited to, using a PCR-assisted mutagenesis technique. Mutations that consist of deletions or additions of amino acid residues to an IL-2 polypeptide can also be made with standard recombinant techniques. In the event of a deletion or addition, the nucleic acid molecule encoding IL-2 is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

An IL2 mutein of the present disclosure may be produced recombinantly not only directly, but also as a fusion polypeptide with a heterologous polypeptide, e.g., a signal sequence or other polypeptide having a specific cleavage site at the N-terminus or C-terminus of the mature IL2 mutein. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In some embodiments, the signal sequence is the signal sequence that is natively associated with the IL2 mutein (i.e., the human IL2 signal sequence). The inclusion of a signal sequence depends on whether it is desired to secrete the αβhIL2 mutein from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild type IL-2 signal sequence be used. Alternatively, heterologous mammalian signal sequences may be suitable, such as signal sequences from secreted polypeptides of the same or related species, as well as viral secretory leaders, for example, the herpes simplex gD signal. When the recombinant host cell is a yeast cell such as Saccharomyces cerevisiae, the alpha mating factor secretion signal sequence may be employed to achieve extracellular secretion of the IL2 mutein into the culture medium as described in Singh, U.S. Pat. No. 7,198,919 B1 issued Apr. 3, 2007.

In the event the IL2 mutein to be expressed is to be expressed as a chimera (e.g., a fusion protein comprising an IL2 mutein and a heterologous polypeptide sequence), the chimeric protein can be encoded by a hybrid nucleic acid molecule comprising a first sequence that encodes all or part of the αβhIL2 mutein and a second sequence that encodes all or part of the heterologous polypeptide. For example, subject αβhIL2 muteins described herein may be fused to a hexa-histidine tag to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the IL2 mutein. For example, the N-terminus may be linked to a targeting domain and the C-terminus linked to a hexa-histidine tag purification handle.

The complete amino acid sequence of the polypeptide (or fusion/chimera) to be expressed can be used to construct a back-translated gene. A DNA oligomer containing a nucleotide sequence coding for αβhIL2 mutein can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the IL2 mutein may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g., Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Expression Vectors:

Once assembled (by synthesis, site-directed mutagenesis or another method), the nucleic acid sequence encoding an αβhIL2 mutein will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker:

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences:

Expression vectors for αβκ of the present disclosure contain a regulatory sequence that is recognized by the host organism and is operably linked to nucleic acid sequence encoding the IL2 mutein. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject αβhIL2 mutein, particularly as regards potential secondary structures.

### Promoters:

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers:

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells:

The present disclosure further provides prokaryotic or eukaryotic cells that contain and express a nucleic acid molecule that encodes a αβhIL2 mutein. A cell of the present disclosure is a transfected cell, i.e., a cell into which a nucleic acid molecule, for example a nucleic acid molecule encoding a mutant IL-2 polypeptide, has been introduced by means of recombinant DNA techniques. The progeny of such a cell are also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant αβhIL2 muteins or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif), and pPicZ (Invitrogen Corporation, San Diego, Calif); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The αβhIL2 mutein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, αβhIL2 muteins obtained will be glycosylated or unglycosylated depending on the host organism used to produce the mutein. If bacteria are chosen as the host then the αβhIL2 mutein produced will be unglycosylated. Eukaryotic cells, on the other hand, will glycosylate the αβhIL2 muteins, although perhaps not in the same way as native-IL-2 is glycosylated.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection:

The expression constructs of the can be introduced into host cells to thereby produce the αβhIL2 muteins disclosed herein or to produce biologically active muteins thereof. Vector DNA can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins:

Recombinantly produced IL2 mutein polypeptides can be recovered from the culture medium as a secreted polypeptide if a secretion leader sequence is employed. Alternatively, the IL2 mutein polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

### Purification:

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

The αβhIL2 mutein produced by the transformed host can be purified according to any suitable method. Various methods are known for purifying IL-2. See, e.g., Current Protocols in Protein Science, Vol 2. Eds: John E. Coligan, Ben M. Dunn, Hidde L. Ploehg, David W. Speicher, Paul T. Wingfield, Unit 6.5 (Copyright 1997, John Wiley and Sons, Inc.). αβhIL2 muteins can be isolated from inclusion bodies generated in E. coli, or from conditioned medium from either mammalian or yeast cultures producing a given mutein using cation exchange, gel filtration, and or reverse phase liquid chromatography.

The substantially purified forms of the recombinant polypeptides can be purified from the expression system using routine biochemical procedures, and can be used, e.g., as therapeutic agents, as described herein.

The biological activity of the αβhIL2 muteins can be assayed by any suitable method known in the art and may be evaluated as substantially purified forms or as part of the cell lysate or cell medium when secretion leader sequences are employed for expression. Such activity assays include CTLL-2 proliferation, induction of phospho-STAT5 (pSTAT5) activity in T cells, PHA-blast proliferation and NK cell proliferation.

### Pharmaceutical Formulations

In some embodiments of the therapeutic methods of the present disclosure involve the administration of a pharmaceutical formulation comprising an αβhIL2 mutein (and/or nucleic acids encoding the αβhIL2 mutein) to a subject in need of treatment. Administration to the subject may be achieved by intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The αβhIL2 muteins also are suitably administered by intratumoral, peritumoral, intralesional, intranodal or perilesional routes or to the lymph, to exert local as well as systemic therapeutic effects.

In some embodiments, the subject αβhIL2 mutein (and/or nucleic acids encoding the αβhIL2 mutein) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the αβhIL2 mutein is to be administered to the subject in need of treatment or prophyaxis.

Parenteral Formulations: In some embodiments, the methods of the present disclosure involve the parental administration of an αβhIL2 mutein. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion.

Carriers: Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor EL™ (BASF, Parsippany, N.J.) or phosphate buffered saline (PBS).

Buffers: The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

Dispersions: Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Preservatives: The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

Tonicity Agents: In many cases, it will be preferable to include isotonic agents, for example, sugars, polyalcohols such as mannitol, sorbitol, sodium chloride in the composition.

Oral Compositions: Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

Inhalation Formulations: In the event of administration by inhalation, subject αβhIL2 muteins, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

Mucosal and Transdermal: Systemic administration of the subject αβhIL2 muteins or nucleic acids can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

Extended Release and Depot Formulations: In some embodiments of the method of the present disclosure, the αβhIL2 mutein is administered to a subject in need of treatment in a formulation to provide extended release of the αβhIL2 mutein agent. Examples of extended release formulations of the injectable compositions can be brought about by including in the composition an agent which delays absorption, for example, aluminum monostearate and gelatin. In one embodiment, the subject αβhIL2 muteins or nucleic acids are prepared with carriers that will protect the mutant IL-2 polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Administration of Nucleic Acids Encoding the IL2 Mutein:

In some embodiments of the method of the present disclosure, nucleic acids encoding the αβhIL2 mutein are administered to the subject by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996). In some embodiments, the αβhIL2 mutein is administered to a subject by the administration of a pharmaceutically acceptable formulation of recombinant expression vector. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in lymphocytes.

In one embodiment, the αβhIL2 mutein formulation is provided in accordance with the teaching of Fernandes and Taforo, U.S. Pat. No. 4,604,377 issued Aug. 5, 1986, the teaching of which is herein incorporated by reference, and Yasui, et al., U.S. Pat. No. 4,645,830.

The parenteral preparation can be enclosed in ampoules, disposable syringes or multiple dose vials made of glass or plastic. In one embodiment, the formulation is provided in a prefilled syringe for parenteral administration.

### Methods of Use

The present disclosure provides methods of use of αβhIL2 muteins, ex vivo and/or in vivo, in combination with TIL therapy for the treatment of subjects suffering from a neoplastic disease disorder or condition.

In some embodiments, the disclosure provides methods and compositions for the treatment and/or prevention of neoplastic diseases, disorders or conditions by the administration a population T cells enriched CD8+ CD25+ T cells in combination with a therapeutically effective amount of αβhIL2 mutein, optionally in combination with one or more supplementary agents, including but not limited to one or more of chemotherapeutics, immune checkpoint modulators, radiotherapy and/or physical interventional treatment methods such as surgery.

In some embodiments of the invention the present disclosure provides methods and compositions for the treatment and/or prevention of neoplastic diseases, disorders or conditions by the administration a TIL cell product enriched CD8+ CD25+ T cells in combination with a therapeutically effective amount of an αβhIL2 mutein wherein the serum concentration of the αβhIL2 mutein is maintained for a majority (i.e., greater than about 50% of the period of time, alternatively greater than about 60%, alternatively greater than about 70%, alternatively greater than about 80%, alternatively greater than about 90%) of a period of time (e.g. at least 24 hours, alternatively at least 48 hours, alternatively at least 72 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer) at a serum concentration at or above the effective concentration of the αβhIL2 mutein sufficient to promote proliferation of CD3− activated primary human T-cells (e.g., at or above EC₁₀^{PRO}, alternatively at or above EC₂₀^{PRO} alternatively at or above EC₃₀^{PRO}, alternatively at or above EC₄₀^{PRO}, at or above EC₅₀^{PRO} alternatively at or above EC₆₀^{PRO}) with respect to such αβhIL2 mutein and at a serum concentration at or above of the effective concentration at a serum concentration of such αβhIL2 mutein sufficient to induce activation of T-cells (e.g., at or above EC₇₀^{ACT}alternatively at or above EC₅₀^{ACT}, alternatively at or above EC₄₀^{A}, at or above EC₃₀^{1T} at at or above EC₂₀^{ACT}, alternatively at or above EC₁₀^{ACT}) with respect to such αβhIL2 mutein.

### Neoplasms Amenable to Treatment:

The compositions and methods of the present disclosure are useful in the treatment of subject suffering from a neoplastic disease. As previously described, TILs have been recovered from a wide variety of human tumors including but not limited cervical cancer (Stevanovic, et al. (2015) J Clin Oncol 33:1543-1550), renal cell cancer (Andersen, et al. (2018) Cancer Immunol Res 6:222-235), breast cancer (Lee, et al. (2017) Oncotarget 8:113345-113359), non-small cell lung cancer (Ben-Avi, et al. (2018) Cancer Immunol Immunotherapy 67:1221-1230) gastrointestinal cancers (Turcotte (2013) J Immunol 191:2217-2225 and Turcotte et al (2014) Clin Cancer Res 20:331-343), cholangiocarcinoma (Tran, et al. (2014) Science 344:641-645), pancreatic cancer (Hall, et al. (2016) J Immunother Cancer 4:61) head and neck cancer (Junker, et al. (2011) Cytotherapy 13:822-834) and ovarian cancer (Fujita, et al. (1995) Clin Cancer Res 1: 501-507).

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

In some embodiments, the neoplastic disease is characterized by the presence of neoplasms, including benign neoplasms. Examples benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

### Assessing Anti-Neoplastic Efficacy:

The determination of efficacy of the methods of the present disclosure in the treatment of cancer is generally associated with the achievement of one or more art recognized parameters such as reduction in lesions particularly reduction of metastatic lesion, reduction in metastatsis, reduction in tumor volume, improvement in ECOG score, and the like. Determining response to treatment can be assessed through the measurement of biomarker that can provide reproducible information useful in any aspect of αβhIL2 mutein therapy, including the existence and extent of a subject's response to such therapy and the existence and extent of untoward effects caused by such therapy. By way of example, but not limitation, biomarkers include enhancement of IFNy, and upregulation of granzyme A, granzyme B, and perform; increase in CD8+ T-cell number and function; enhancement of IFNγ, an increase in ICOS expression on CD8+ T-cells, enhancement of IL-10 expressing T_{Reg} cells. The response to treatment may be characterized by improvements in conventional measures of clinical efficacy may be employed such as Complete Response (CR), Partial Response (PR), Stable Disease (SD) and with respect to target lesions, Complete Response (CR),” Incomplete Response/Stable Disease (SD) as defined by RECIST as well as immune-related Complete Response (irCR), immune-related Partial Response (irPR), and immune-related Stable Disease (irSD) as defined Immune-Related Response Criteria (irRC) are considered by those of skill in the art as evidencing efficacy in the treatment of neoplastic disease in mammalian (e.g. human) subjects.

Further embodiments comprise a method or model for determining the optimum amount of an agent(s) in a combination. An optimum amount can be, for example, an amount that achieves an optimal effect in a subject or subject population, or an amount that achieves a therapeutic effect while minimizing or eliminating the adverse effects associated with one or more of the agents. In some embodiments, the methods involving the combination of an αβhIL2 mutein and a supplementary agent which is known to be, or has been determined to be, effective in treating or preventing a disease, disorder or condition described herein (e.g., a cancerous condition) in a subject (e.g., a human) or a subject population, and an amount of one agent is titrated while the amount of the other agent(s) is held constant. By manipulating the amounts of the agent(s) in this manner, a clinician is able to determine the ratio of agents most effective for, for example, treating a particular disease, disorder or condition, or eliminating the adverse effects or reducing the adverse effects such that are acceptable under the circumstances.

Combination TIL Therapy and αβhIL2 Muteins with Additional Supplementary Therapeutic Agents:

During the in vivo phase of treatment with, the αβhIL2 mutein used to support the activated T cells administered to the subject may be administered in further combination with one or more supplementary agents useful in the treatment of neoplastic disease as described below.

The use of αβhIL2 muteins, ex vivo and/or in vivo, in combination with TIL therapy as provided in present disclosure may be further employed in combination with one or more additional active agents (“supplementary agents”). Such further combinations are referred to interchangeably as “supplementary combinations” or “supplementary combination therapy” and those therapeutic agents that are used in combination with use of αβhIL2 muteins, ex vivo and/or in vivo, in combination with TIL therapy are referred to herein as “supplementary agents.” As used herein, the term “supplementary agents” includes agents that can be administered or introduced separately, for example, formulated separately for separate administration (e.g., as may be provided in a kit) and/or therapies that can be administered or introduced in combination with the αβhIL2 muteins, ex vivo and/or in vivo, in combination with TIL therapy.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g. αβhIL2 muteins) is considered to be administered in combination with a second agent (e.g. a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g., nivolumab or pembrolizumab) are typically administered by IV infusion every two weeks or every three weeks while the αβhIL2 mutein of the present disclosure are typically administered more frequently, e.g., daily, BID, or weekly. However, the administration of the first agent (e.g. pembrolizumab) provides a therapeutic effect over an extended time and the administration of the second agent (e.g. an αβhIL2 muteins) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the αβhIL2 mutein and the supplementary agent(s) are administered or applied sequentially to the subject, e.g., where one agent is administered prior to one or more other agents. In other embodiments, the αβhIL2 mutein and the supplementary agent(s) are administered simultaneously to the subject, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

### Chemotherapeutic Agents:

In some embodiments, the supplementary agent is a chemotherapeutic agent. In some embodiments the supplementary agent is a “cocktail” of multiple chemotherapeutic agents. IN some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent is one or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti- tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foreoing as practied in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the αβhIL2 mutein is administered to the subject in vivo in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

### Therapeutic Antibodies

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g., trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Le^{y}, VEGF (e.g. bevacizumab), VEGFR, VEGFR2 (e.g. ramucirumab), PDGFRα (e.g. olartumumab), EGFR (e.g. cetuximab, panitumumab and necitumumab), ERBB2 (e.g. trastuzumab), ERBB3, MET, IGF1R, EPHA3, TRAIL RI, TRAIL R2, RANKL RAP, tenascin, integrin aVP3, and integrin a4s1.

Examples of antibody therapeutics which are FDA approved and may be used as supplementary agents for use in the treatment of neoplastic disease indicateion include those provided in Table XXX below.

TABLE XXX
FDA Antineoplastic Disease Antibodies and Indications

| Name | Tradename(s) | Target; format | Indication |
|---|---|---|---|
| [fam]- | Enhertu | HER2; Humanized IgG1 | HER2+ breast cancer |
| trastuzumab | | ADC | |
| deruxtecan | | | |
| Enfortumab | Padcev | Nectin-4; Human IgG1 ADC | Urothelial cancer |
| vedotin | | | |
| Polatuzumab | Polivy | CD79b; Humanized IgG1 | Diffuse large B-cell lymphoma |
| vedotin | | ADC | |
| Cemiplimab | Libtayo | PD-1; Human mAb | Cutaneous squamous cell |
| | | | carcinoma |
| Moxetumomab | Lumoxiti | CD22; Murine IgG1 dsFv | Hairy cell leukemia |
| pasudotox | | immunotoxin | |
| Mogamuizumab | Poteligeo | CCR4; Humanized IgG1 | Cutaneous T cell lymphoma |
| Tildrakizumab | Ilumya | IL-23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, | Hematological malignancy |
| ozogamicin | | ADC | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Ixekizumab | Taltz | IL-17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Nivolumab | Opdivo | PD1; Human IgG4 | Melanoma, non-small cell lung |
| | | | cancer |
| Blinatumomab | Blincyto | CD19, CD3; Murine | Acute lymphoblastic leukemia |
| | | bispecific tandem scFv | |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL-6; Chimeric IgG1 | Castleman disease |
| Obinutuzumab | Gazyva | CD20; Humanized IgG1; | Chronic lymphocytic leukemia |
| | | Glycoengineered | |
| Ado-trastuzumab | Kadcyla | HER2; Humanized IgG1, | Breast cancer |
| emtansine | | ADC | |
| Pertuzumab | Perjeta | HER2; Humanized IgG1 | Breast Cancer |
| Brentuximab | Adcetris | CD30; Chimeric IgG1, ADC | Hodgkin lymphoma, systemic |
| vedotin | | | anaplastic large cell lymphoma |
| Ipilimumab | Yervoy | CTLA-4; Human IgG1 | Metastatic melanoma |
| Ofatumumab | Arzerra | CD20; Human IgG1 | Chronic lymphocytic leukemia |
| Certolizumab | Cimzia | TNF; Humanized Fab, | Crohn disease |
| pegol | | pegylated | |
| Catumaxomab | Removab | EPCAM/CD3; Rat/mouse | Malignant ascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab- | Bexxar | CD20; Murine IgG2a | Non-Hodgkin lymphoma |
| I131 | | | |
| Ibritumomab | Zevalin | CD20; Murine IgG1 | Non-Hodgkin lymphoma |
| tiuxetan | | | |
| Gemtuzumab | Mylotarg | CD33; Humanized IgG4, | Acute myeloid leukemia |
| ozogamicin | | ADC | |
| Trastuzumab | Herceptin | HER2; Humanized IgG1 | Breast cancer |
| Infliximab | Remicade | TNF; Chimeric IgG1 | Crohn disease |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

In some embodiments, where the antibody is a bispecific antibody targeting a first and second tumor antigen such as HER2 and HER3 (abbreviated HER2×HER3), FAP x DR-5 bispecific antibodies, CEA×CD3 bispecific antibodies, CD20×CD3 bispecific antibodies, EGFR-EDV-miR16 trispecific antibodies, gplOO×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PDT×CTLA-4 bispecific antibodies, CD123×CD3 bispecific antibodies, gpA33×CD3 bispecific antibodies, B37-H-3×CD3 bispecific antibodies, LAG-3×PDT bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies,

CD20×CD3 bispecific antibodies, CD123×CD3 bispecific antibodies, SSTR2 X CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies.

Such therapeutic antibodies may be further conjugated to one or more chemotherapeutic agents (e.g antibody drug conjugates or ADCs) directly or through a linker, especially acid, base or enzymatically labile linkers.

### Physical Methods:

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising an IL2 mutein and one or more supplementary agents. In some embodiments, the present disclosure further contemplates the use of an IL2 mutein in combination with surgery (e.g. tumor resection). In some embodiments, the present disclosure further contemplates the use of an IL2 mutein in combination with bone marrow transplantation, peripheral blood stem cell transplantation or other types of transplantation therapy.

### Immune Checkpoint Modulators:

In some embodiments, a “supplementary agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

An immune checkpoint whose activation results in inhibition or downregulation of the immune response is referred to herein as a “negative immune checkpoint pathway modulator.” The inhibition of the immune response resulting from the activation of a negative immune checkpoint modulator diminishes the ability of the host immune system to recognize foreign antigen such as a tumor-associated antigen. The term negative immune checkpoint pathway includes, but is not limited to, biological pathways modulated by the binding of PD1 to PDL1, PD1 to PDL2, and CTLA4 to CDCD80/86. Examples of such negative immune checkpoint antagonists include but are not limited to antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152).

In one embodiment, an immune checkpoint pathway the activation of which results in stimulation of the immune response is referred to herein as a “positive immune checkpoint pathway modulator.” The term positive immune checkpoint pathway modulator includes, but is not limited to, biological pathways modulated by the binding of ICOSL to ICOS(CD278), B7-H6 to NKp30, CD155 to CD96, OX40L to OX40, CD70 to CD27, CD40 to CD40L, and GITRL to GITR. Molecules which agonize positive immune checkpoints (such natural or synthetic ligands for a component of the binding pair that stimulates the immune response) are useful to upregulate the immune response. Examples of such positive immune checkpoint agonists include but are not limited to agonist antibodies that bind T-cell activating receptors such as ICOS (such as JTX- 2011, Jounce Therapeutics), OX40 (such as MED16383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up- regulating at least partially the function of a stimulatory immune checkpoint (referred to herein as an “immune checkpoint pathway effector” or “immune checkpoint pathway agonist.”).

The immune response mediated by immune checkpoint pathways is not limited to T-cell mediated immune response. For example, the KIR receptors of NK cells modulate the immune response to tumor cells mediated by NK cells. Tumor cells express a molecule called HLA-C, which inhibits the KIR receptors of NK cells leading to a dimunition or the anti-tumor immune response. The administration of an agent that antagonizes the binding of HLA-C to the KIR receptor such an anti-KIR3 mab (e.g. lirilumab, BMS) inhibits the ability of HLA-C to bind the NK cell inhibitory receptor (KIR) thereby restoring the ability of NK cells to detect and attack cancer cells. Thus, the immune response mediated by the binding of HLA-C to the KIR receptor is an example a negative immune checkpoint pathway the inhibition of which results in the activation of a of non-T-cell mediated immune response.

In one embodiment, the immune checkpoint pathway modulator is a negative immune checkpoint pathway inhibitor/antagonist. In another embodiment, immune checkpoint pathway modulator employed in combination with the IL2 mutein is a positive immune checkpoint pathway agonist. In another embodiment, immune checkpoint pathway modulator employed in combination with the IL2 mutein is an immune checkpoint pathway antagonist.

The term “negative immune checkpoint pathway inhibitor” refers to an immune checkpoint pathway modulator that interferes with the activation of a negative immune checkpoint pathway resulting in the upregulation or enhancement of the immune response. Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

The term PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline. Aptamer compounds are also described in the literature useful as PD1 pathway inhibitors (Wang, et al. (2018) 145:125-130.).

The term PD1 pathway inhibitors includes peptidyl PD1 pathway inhibitors such as those described in Sasikumar, et al., U.S. Pat. No. 9,422,339 issued Aug. 23, 2016, and Sasilkumar, et al., U.S. Pat. No. 8,907,053 issued Dec. 9, 2014. CA-170 (AUPM-170, Aurigene/Curis) is reportedly an orally bioavailable small molecule targeting the immune checkpoints PDL1 and VISTA. Pottayil Sasikumar, et al. Oral immune checkpoint antagonists targeting PD-L1/VISTA or PD-L1/Tim3 for cancer therapy. [abstract]. In: Proceedings of the 107th Annual Meeting of the American Association for Cancer Research; 2016 Apr. 16-20; New Orleans, LA. Philadelphia (PA): AACR; Cancer Res 2016; 76(14 Suppl): Abstract No. 4861. CA-327 (AUPM-327, Aurigene/Curis) is reportedly an orally available, small molecule that inhibit the immune checkpoints, Programmed Death Ligand-1 (PDL1) and T-cell immunoglobulin and mucin domain containing protein-3 (TIM3).

The term PD1 pathway inhibitors includes small molecule PD1 pathway inhibitors. Examples of small molecule PD1 pathway inhibitors useful in the practice of the present invention are described in the art including Sasikumar, et al., 1,2, 4-oxadiazole and thiadiazole compounds as immunomodulators (PCT/IB2016/051266 filed Mar. 7, 2016, published as WO2016142833A1 Sep. 15, 2016) and Sasikumar, et al. 3-substituted-1,2, 4-oxadiazole and thiadiazole PCT/IB2016/051343 filed Mar. 9, 2016 and published as WO2016142886A2), BMS-1166 and Chupak L S and Zheng X. Compounds useful as immunomodulators. Bristol-Myers Squibb Co. (2015) WO 2015/034820 A1, EP3041822 B1 granted Aug. 9, 2017; WO2015034820 Al; and Chupak, et al. Compounds useful as immunomodulators. Bristol-Myers Squibb Co. (2015) WO 2015/160641 A2. WO 2015/160641 A2, Chupak, et al. Compounds useful as immunomodulators. Bristol-Myers Squibb Co. Sharpe, et al. Modulators of immunoinhibitory receptor PD-1, and methods of use thereof, WO 2011082400 A2 published Jul. 7, 2011; U.S. Pat. No. 7,488,802 (Wyeth) issued Feb. 10, 2009;

In some embodiments, combination of TIL therapy with αβhIL2 muteins and one or more PD1 immune checkpoint modulators are useful in the treatment of neoplastic conditions for which PD1 pathway inhibitors have demonstrated clinical effect in human beings either through FDA approval for treatment of the disease or the demonstration of clinical efficacy in clinical trials including but not limited to melanoma, non-small cell lung cancer, small cell lung cancer, head and neck cancer, renal cell cancer, bladder cancer, ovarian cancer, uterine endometrial cancer, uterine cervical cancer, uterine sarcoma, gastric cancer, esophageal cancer, DNA mismatch repair deficient colon cancer, DNA mismatch repair deficient endometrial cancer, hepatocellular carcinoma, breast cancer, Merkel cell carcinoma, thyroid cancer, Hodgkins lymphoma, follicular lymphoma, diffuse large B-cell lymphoma, mycosisfungoides, peripheral T-cell lymphoma. In some embodiments, the combination of IL2 muteins and an PD1 immune checkpoint modulator is useful in the treatment of tumors characterized by high levels of expression of PDL1, where the tumor has a tumor mutational burden, where there are high levels of CD8+ T-cell in the tumor, an immune activation signature associated with IFNγ and the lack of metastatic disease particularly liver metastasis.

In some embodiments, the combination of TIL therapy with αβhIL2 muteins is administered in further combination with an antagonist of a negative immune checkpoint pathway that inhibits the binding of CTLA4 to CD28 (“CTLA4 pathway inhibitor”). Examples of CTLA4 pathway inhibitors are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (Medarex, Inc.) issued Oct. 20, 2009)

In some embodiments, the combination of TIL therapy with αβhIL2 muteins is administered in further combination an antagonist of a negative immune checkpoint pathway that inhibits the binding of BTLA to HVEM (“BTLA pathway inhibitor”). A number of approaches targeting the BTLA/HVEM pathway using anti-BTLA antibodies and antagonistic HVEM-Ig have been evaluated, and such approaches have suggested promising utility in a number of diseases, disorders and conditions, including transplantation, infection, tumor, and autoimmune disease (See e.g. Wu, et al., (2012) Int. J. Biol. Sci. 8:1420-30).

In some embodiments, the combination of TIL therapy with αβhIL2 muteins is administered in further combination with an antagonist of a negative immune checkpoint pathway that inhibits the ability TIM3 to binding to TIM3- activating ligands (“TIM3 pathway inhibitor”). Examples of TIM3 pathway inhibitors are known in the art and with representative non-limiting examples described in United States Patent Publication No. PCT/US2016/021005 published Sep. 15, 2016; Lifke, et al. United States Patent Publication No. US 20160257749 Al published Sep. 8, 2016 (F. Hoffman-LaRoche), Karunsky, U.S. Pat. No. 9,631,026 issued Apr. 27, 2017; Karunsky, Sabatos-Peyton, et al. U.S. Pat. No. 8,841,418 issued Sep. 23, 2014; U.S. Pat. No. 9,605,070; Takayanagi, et al., U.S. Pat. No. 8,552,156 issued Oct. 8, 2013.

In some embodiments, the combination of TIL therapy with αβhIL2 muteins is administered in further combination with an inhibitor of both LAG3 and PD1 as the blockade of LAG3 and PD1 has been suggested to synergistically reverse anergy among tumor-specific CD8+ T-cells and virus-specific CD8+ T-cells in the setting of chronic infection. IMP321 (ImmuFact) is being evaluated in melanoma, breast cancer, and renal cell carcinoma. See generally Woo et al., (2012) Cancer Res 72:917-27; Goldberg et al., (2011) Curr. Top. Microbiol. Immunol. 344:269-78; Pardoll (2012) Nature Rev. Cancer 12:252-64; Grosso et al., (2007) J. Clin. Invest. 117:3383-392].

In some embodiments, the combination of TIL therapy with αβhIL2 muteins is administered in further combination with combination with an A2aR inhibitor. A2aR inhibits T-cell responses by stimulating CD4+ T-cells towards developing into T_{Reg} cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the TIL therapy with αβhIL2 muteins for use in the treatment disorders such as cancer and Parkinson's disease.

In some embodiments, the combination of TIL therapy with αβhIL2 muteins is administered in further combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g. cancer) in a mammalian subject by the administration In the combination of TIL therapy with αβhIL2 muteins is administered in further combination with an agent(s) that modulate at least one immune checkpoint pathway including immune checkpoint pathway modulators that modulate two, three or more immune checkpoint pathways.

In some embodiments, the combination of TIL therapy with αβhIL2 muteins is administered in further combination with an immune checkpoint modulator that is capable of modulating multiple immune checkpoint pathways. Multiple immune checkpoint pathways may be modulated by the administration of multi-functional molecules which are capable of acting as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of TIL therapy with αβhIL2 muteins in further combination with muteins in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the combination of TIL therapy with αβhIL2 muteins is administered in further combination with combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a IL2 mutein of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

### Chemokine and Cytokine Agents as Supplementary Agents:

In some embodiments, the combination of TIL therapy with αβhIL2 muteins is administered in further combination with additional cytokines including but not limited to IL-7, IL-12, IL-15 and IL-18 including analogs and variants of each thereof.

### Activation-induced Cell Death Inhibitors

In some embodiments, the combination of TIL therapy with αβhIL2 muteins is administered in further combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the IL2 muteins described herein include but are not limited to cyclosporin A (Shih, et al., (1989) Nature 339:625-626, IL-16 and analogs (including rhIL-16, Idziorek, et al., (1998) Clinical and Experimental Immunology 112:84-91), TGFbl (Genesteir, et al., (1999) J Exp Med189(2): 231-239), and vitamin E (Li-Weber, et al., (2002) J Clin Investigation 110(5):681-690).

### Physical Methods

In some embodiments, the combination of TIL therapy with αβhIL2 muteins is administered in further combination with physical methods of the treatment of neoplastic disease including but not limited to radiotherapy, cryotherapy, hyperthermic therapy, surgery, laser ablation, and proton therapy.

### Dosage:

Dosage, toxicity and therapeutic efficacy of such subject αβhIL2 muteins or nucleic acids compounds can be determined by standard pharmaceutical procedures in cell cultures or experimental animals. The data obtained from the cell culture assays and animal studies can be used in formulating a range of dosage for use in humans. The dosage of such compounds lies preferably within a range of circulating concentrations that include the ED50 with minimal acceptable toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 as determined in cell culture. Such information can be used to more accurately determine useful doses in humans. Levels in plasma may be measured, for example, by high performance liquid chromatography.

As defined herein, a therapeutically effective amount of a subject αβhIL2 muteins (i.e., an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU) as established by the World Health Organization 1st International Standard for Interleukin-2 (human)).

In some embodiments of the invention the present disclosure provides methods and compositions for the treatment and/or prevention of neoplastic diseases, disorders or conditions in a subject by the administration to the subject a therapeutically effective amount of an αβhIL2 mutein of the present disclosure wherein the serum concentration of is maintained for a majority (i.e., greater than about 50% of the period of time, alternatively greater than about 60%, alternatively greater than about 70%, alternatively greater than about 80%, alternatively greater than about 90%) of a period of time (e.g. at least 24 hours, alternatively at least 48 hours, alternatively at least 72 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer) at a serum concentration at or above the effective concentration of the αβhIL2 mutein sufficient to promote proliferation of CD3-activated primary human T-cells (e.g., at or above EC₁₀^{PRO}, alternatively at or above EC₂₀^{PRO}, alternatively at or above EC₃₀^{PRO}, alternatively at or above EC₄₀^{PRO}, at or above EC₅₀^{PRO}, alternatively at or above EC₆₀^{PRO}) with respect to such αβhIL2 mutein but at a serum concentration at or below of the effective concentration at a serum concentration of such IL2 mutein sufficient to induce activation of T-cells (e.g, at or below EC₁₀₀^{PRO}, alternatively at or below EC₉₀^{PRO} alternatively at or below EC₅₀^{PRO}, alternatively at or below EC₇₀^{PRO}, at or below EC₆₀^{PR}O alternatively at or below EC₅₀^{PRO}) with respect to such αβhIL2 mutein.

In some embodiments of the invention the present disclosure provides methods and compositions for the treatment and/or prevention of neoplastic diseases, disorders or conditions in a subject by the administration to the subject wherein a therapeutically effective amount of an human IL-2 mutein sufficient to maintain a serum concentration of the αβhIL2 mutein at or above the effective concentration of the IL2 mutein sufficient to promote proliferation of CD3-activated primary human T-cells (>EC₁₀^{PRO}) and at or below a serum concentration of the αβhIL2 mutein sufficient to induce activation of T-cells with respect to such IL2 mutein (i.e. below EC₉₀^{PRO}) for more than about 50%, alternatively greater than about 60%, alternatively greater than about 70%, alternatively greater than about 80%, alternatively greater than about 90%) of a period of time of at least 24 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer.

In some embodiments the present disclosure provides methods and compositions for the treatment and/or prevention of neoplastic diseases, disorders or conditions in a subject by the administration to the subject wherein a therapeutically effective amount of the αβhIL2 mutein sufficient to maintain a serum concentration of human said IL2 mutein at or above the effective concentration of the IL2 mutein sufficient to promote proliferation of CD3-activated primary human T-cells (>EC₁₀^{PRO}) and at or below a serum concentration of the αβhIL2 mutein sufficient to induce activation of T-cells with respect to the αβhIL2 mutein (i.e. below EC₉₀^{PRO}) for more than about 50%, alternatively greater than about 60%, alternatively greater than about 70%, alternatively greater than about 80%, alternatively greater than about 90%) of a period of time of at least 24 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer wherein th the αβhIL2 mutein comprises a set of mutations selected from the group consisting of the following sets of mutations: L18R, Q22E, and Q126H; L18R, Q22E, and Q126K; L18R, Q22E and Q126M; L18R, Q22E Q126T; L18R; Q22E; V91K; V91R; Q126H; L18R, and Q126H; Q22E, and Q126H; L18G, Q22E and Q126H; L18A, Q22E and Q126H; L18M, Q22E and Q126H; L18F, Q22E and Q126H; L18W, Q22E and Q126H; L18K, Q22E and Q126H; L18Q, Q22E and Q126H; L18E, Q22E and Q126H; L18S, Q22E and Q126H; L18V, Q22E and Q126H; L181, Q22E and Q126H; L18Y, Q22E and Q126H; L18H, Q22E and Q126H; L18N, Q22E and Q126H; L18D, Q22E and Q126H; L18T, Q22E and Q126H; L18R, Q22G and Q126H; L18R, Q22A and Q126H; L18R, Q22L and Q126H; L18R, Q22M and Q126H; L18R, Q22F and Q126H; L18R, Q22W and Q126H; L18R, Q22K and Q126H; L18R, Q22S and Q126H; L18R, Q22V and Q126H; L18R, Q221 and Q126H; L18R Q22Y and Q126H; L18R Q22H and Q126H; L18R Q22R and Q126H; L18R Q22N and Q126H; L18R Q22D and Q126H; and L18R Q22T and Q126H.

In accordance with another aspect of the present invention, there is provided a method for stimulating the immune system of an animal by administering the αβhIL2 mutein of the present disclosure prior to the isolation of the TILs from the subject. The method is useful to treat disease states where the host immune response is deficient. In treating a subject, a therapeutically effective dose of the αβhIL2 mutein is administered. A therapeutically effective dose refers to that amount of the active ingredient that produces amelioration of symptoms or a prolongation of survival of a subject. An effective dose will vary with the characteristics of the αβhIL2 mutein to be administered, the physical characteristics of the subject to be treated, the nature of the disease or condition, and the like. A single administration can range from about 50,000 IU/kg to about 1,000,000 IU/kg or more, more typically about 600,000 IU/kg. This may be repeated several times a day (e.g., 2-3 times per day) for several days (e.g., about 3-5 consecutive days) and then may be repeated one or more times following a period of rest (e.g., about 7-14 days). Thus, an effective dose may comprise only a single administration or many administrations over a period of time (e.g., about 20-30 individual administrations of about 600,000 IU/kg each given over about a 10-20 day period).

The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the subject the αβhIL2 mutein can include a single treatment or, can include a series of treatments. In one embodiment, the compositions are administered every 8 hours for five days, followed by a rest period of 2 to 14 days, e.g., 9 days, followed by an additional five days of administration every 8 hours. In another embodiment, the compositions are administered every other day for a period of at least 6 days, optionally at least 10 days, optionally at least 14 days, optionally at least 30 days, optionally at least 60 days. The skilled artisan will recognize that the treatment may be extended for the treatment of chronic conditions and the prevent the reoccurrence of symptoms of chronic diseases such as autoimmune diseases (e.g., psoriasis, IBD, etc.) to selectively or preferentially activate engineered Tregs both ex vivo and/or in vivo.

The pharmaceutical compositions comprising the αβhIL2 mutein can be included in a container, pack, or dispenser together with instructions for administration.

While compounds that exhibit toxic side effects may be used, care should be taken to design a delivery system that targets such compounds to the site of affected tissue in order to minimize potential damage to uninfected cells and, thereby, reduce side effects. Toxicity and therapeutic efficacy of an IL-2 mutein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LC₅₀/EC₅o. IL-2 muteins that exhibit large therapeutic indices are preferred. The data obtained from these cell culture assays and animal studies can be used in formulating a range of dosages suitable for use in humans. The dosage of such mutants lies preferably within a range of circulating concentrations that include the ED₅₀ with little or no toxicity. The dosage may vary within this range depending upon a variety of factors, e.g., the dosage form employed, the route of administration utilized, the condition of the subject, and the like.

A therapeutically effective dose can be estimated initially from cell culture assays by determining an EC₅₀. A dose can then be formulated in animal models to achieve a circulating plasma concentration range that includes the EC₅₀ as determined in cell culture. Such information can be used to more accurately determine useful doses in humans. Levels in plasma may be measured, for example, by HPLC. The exact formulation, route of administration and dosage can be chosen by the individual physician in view of the patient's condition.

The attending physician for patients treated with the αβhIL2 mutein would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Kits

The present disclosure also contemplates kits comprising pharmaceutical compositions of a TIL cell product and αβhIL2 mutein. The kits are generally in the form of a physical structure housing various components, as described below, and can be utilized, for example, in practicing the methods described above. A kit may comprise an TIL cell product and αβhIL2 mutein in the form of a pharmaceutical composition suitable for administration to a subject that is ready for use or in a form or requiring preparation for example, thawing, reconstitution or dilution prior to administration. When the TIL cell product or αβhIL2 mutein is in a form that needs to be reconstituted by a user, the kit may also comprise a sterile container providing a reconstitution medium comprising buffers, pharmaceutically acceptable excipients, and the like. A kit of the present disclosure can be designed for conditions necessary to properly maintain the components housed therein (e.g., refrigeration or freezing). A kit may further contain a label or packaging insert including identifying information for the components therein and instructions for their use. Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial). Labels or inserts may be provided in a physical form or a computer readable medium. In some embodiments, the actual instructions are not present in the kit, but rather the kit provides a means for obtaining the instructions from a remote source, e.g., via an internet site, including by secure access by providing a password (or scannable code such as a barcode or QR code on the container of the IL2 mutein or kit comprising) in compliance with governmental regulations (e.g., HIPAA) are provided.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure and are to be included within the spirit and purview of this application.

### EXAMPLES

The following examples are provided to describe certain embodiments of the invention provided herein and are not to be construed to as limiting.

### Example 1. MC38 Tumor Implantation

MC38 tumor cells were grown and maintained at a semi-confluent density at 37° C. in complete DMEM (Gibco, 11995065): 10% FBS (Corning, 35010CV)+1% Penicillin/Streptomycin (Gibco, 15140122). On the day of implantation, MC38 cells were removed from flasks using TrypLE Express (12604039) for 5-6 minutes and counted. MC38 cells were mixed 1:1 with Matrigel Matrix (Corning, 356237) prior to implantation, then 1e6 cells were implanted subcutaneously on the right flank. Once MC38 tumors reached ˜100 mm³, tumors were harvested for further processing.

### Example 2. Tumor Infiltrating T Cell Preparation

MC38 tumors were harvested, and 3-4 tumors were placed into C-tubes (Milteyni Biotech, 130-093-237). Once all tumors were collected in C-tubes, the tumors were manually chopped using scissors. Tumors were enzymatically digested: 40 μg/mL Liberase (Sigma Aldrich, 05401127001)+250 μg/mL DNase (Millipore, 260913) using the 37C_m_TKD_1 protocol on an Octo Dissociator (Milteyni Biotech, 130-096-427). After the incubation, 5 mM EDTA (Invitrogen, 15575-038) was added for 5 minutes. Cells were then filtered, spun, and counted. CD4+/CD8+ T cells were then isolated using the CD4/CD8 TIL microbeads kit (Miltenyi Biotec, 130-116-480). After enrichment, cells were centrifuged and counted again.

### Example 3. Tumor Infiltrating T Cell Expansion

After CD4/CD8 enrichment, cells were plated at 37° C. at 1e6 cells/mL in the presence of either: 25 nM mouse WT IL2+15 nM mouse WT IL10 or 25 nM REH+15 nM mouse WT IL10. Cells were counted and split (cell concentration adjusted to 1e6/mL on day 3 for day 5 harvest) (FIG. 1). Cells were counted and split (cell concentration adjusted to 1e6/mL on days 3 & 5 for day 7 harvest) (FIG. 2). Fresh cytokines were added whenever cells were split.

### Example 4. Tumor Cell IFN-Gamma Treatment

MC38 tumor cells were grown and maintained at a semi-confluent density at 37° C. in complete DMEM: 10% FBS+1% Penicillin/Streptomycin. B16 tumor cells were grown and maintained at a semi-confluent density at 37° C. in complete RPMI (Gibco, 11875093): 10% FBS+1% Penicillin/Streptomycin.

To upregulate appropriate molecules on tumor target cells (major histocompatibility complexes), MC38 & B16 tumor cells were treated with culture media containing IFN-gamma for 48 hours prior to co-culture with tumor infiltrating T cells: 0.5% FBS+1% Penicillin/Streptomycin+1 nM mouse IFN-gamma.

### Example 5. T Cell+Tumor Target Cell Co-Culture Assay Setup

After 5 or 7 days (as stated above), T cells were harvested, centrifuged, and counted. MC38 tumors cells were used as the target cell line expressing cognate tumor antigens, while B16 were used as a strain matched C57BL/6 negative control. IFN-gamma pre-treated B16 and MC38 cells were removed from flasks using TrypLE Express. Target cells were spun and counted. Based on T cell and target cell counts, a ratio of 1:4 (Effector T cells: Target cells) was plated. In this experiment, 12,500 T cells+50,000 target cells were plated in triplicate. Cells were incubated with target cells overnight. ˜16 hours later, 1× monensin (Invitrogen, 00450551) was added for 4-5 hours.

### Example 6. Flow Cytometry Staining+Analyses

After the monensin incubation, cells were spun and washed with PBS. Cells were then stained with fixable viability dye (Invitrogen, 65086614) at 4° C. for 30 minutes. Cells were spun and washed with staining buffer (BD Biosciences, 554656). Cells were then incubated with Fc block (BD Biosciences, 553142) for 10 minutes at 4° C. before addition of anti-CD3 (Biolegend, 100306), anti-CD4 (Biolegend, 100540) & anti-CD8 (BD Biosciences, 563046) for 20 minutes at 4° C. After the incubation, cells were spun and washed with staining buffer then fixed/permed (Invitrogen, 005012343, 0050522356) for 30 minutes at 4° C. After the incubation, cells were spun and washed with staining buffer then stained intracellularly with anti-IFN-gamma (BD Biosciences, 554412) for 20 minutes at 4° C. Cells were then spun, washed, and run on a Cytek Aurora instrument (Fremont, CA).

Flow cytometry analyses were performed using FlowJo (Ashland, OR). Cells were gated following this gating scheme: gate on single cells 4 CD3+4 live/dead dye-→CD4-CD8+→IFN-gamma+. The IFN-gamma+ percentage of CD8+ T cells was graphed using GraphPad Prism (San Diego, CA). The results of these experiments are provided in FIGS. 1 & 2 of the attached drawings. As can be seen, WT mouse IL2 expands a larger population of cells that respond to both MC38 cells and the strain matched B16 cells, whereas REH expands T cells responding to the strain matched B16 cells significantly less (**, p=<0.01, ***=p<0.001).

### Example 7. Evaluation of Binding Affinity Using Surface Plasmon Resonance

All SPR experiments were conducted in 10 mM Hepes, 150 mM NaCl, 0.05% (v/v) Polysorbate 20 (PS20) and 3 mM EDTA (HBS-EP+ buffer) on a Biacore® T200 instrument equipped with anti-histidine CM5, Protein A or CAP biotin chips (Cytiva).

Evaluation of Binding Affinity of α/β-biased hIL2 (αβhIL2) muteins to CD25 Using Surface Plasmon Resonance: Following capture of 40-140 RU of poly histidine-tagged IL-2 muteins on an anti-histidine surface, injections of 2.5, 5, 10 and 20 nM untagged CD25 (R&D Systems, cat #223-2A) were performed in high performance mode (25 second association and 25 second dissociation) followed by surface regeneration with 10 mM glycine-HCl, pH 1.5 (30 seconds, 50 μL/min). Buffer-subtracted sensograms were processed with Biacore T200 Evaluation Software and globally fit with a 1:1 Langmuir binding model (bulk shift set to zero) to extract kinetics and affinity constants (ka, ka, KD). RMAX ranged from 15 to 67 RU, indicating surface density compatible with kinetics analysis.

Evaluation of Binding Affinity of α/β-biased hIL2(αβhIL2) mutein to CD122 Using Surface Plasmon Resonance: With the exception of IL-2-V91K, which was loaded at 1445 RU to rule out lower affinity interactions, 21-156 RU capture of poly histidine-tagged IL-2 mutein on an anti-histidine surface was followed by injections of 125, 250, 500, 1000, 2000 nM untagged IL-2RO/CD122 (Sino Biological, cat #10696-HCCH) performed in low sample consumption mode (30 second association, 60 second dissociation) finally followed by surface regeneration with 10 mM glycine-HCl, pH 1.5 (30 seconds, 50 μL/min). Buffer-subtracted sensograms were processed with Biacore T200 Evaluation Software and fit with steady state analysis. R_{MAX} ranged between 41 and 189 RU.

### Example 8. Ex Vivo TIL Antigen Recall

Tumors were harvested from MC38-tumor bearing mice and single cell suspension were prepared as previously described (27). Immune cells were enriched using Easy Sep™ Mouse TIL (CD45) Positive Selection kit (STEMCELL technologies). Next, CD8+ CD25+ cells were FACS sorted and co-cultured with mouse IFNγ-treated MC38 cells in complete DMEM medium at a 1:2 ratio for 18h. Cytokines in cell supernatants were measured using Proinflammatory mouse U-plex kit (Meso Scale Discovery). Sometimes, cells were treated with Brefeldin A for additional 4 hours, fixed with 1% paraformaldehyde and stained with Biolegend anti-IFNg (clone XMG1.2), anti-Granzyme B (clone GB11) and anti-TNFα (MP6-XT22) antibodies. Cells were acquired on Cytek Aurora (Cytek™)

### Example 9. Flow Cytometry

Antibodies to CD3 (17A2), CD90 (53-2.1), CD8a (53-6.7), CD19 (eBio1D3), CD25 (PC61.5), PD1 (RMP1-30), Ki67 (SolA15) and Foxp3 (FJK-16s) were purchased from eBioscience. Antibodies to CD4 (GK1.5), were purchased from BioLegend. Antibodies to CD45 (30-F11), CD11b (M1/70), Granzyme B (GB11) were purchased from BD. Viability was assessed by Fixable Viability Dye eFluor 506 purchased from eBioscience. Foxp3, Ki67 and Granzyme B were stained using the Foxp3/Transcription Factor Staining Buffer Set from eBioscience. Cells were acquired using a Beckman Coulter Cytoflex cytometer and data was analyzed using FlowJo.

### Example 10a. Cytokine Quantitation

To quantify human IFNγ, TNFα, IL-2, GM-CSF, and mouse IL-1β and IL-6, mouse or cynomolgus macaque sera were subjected to U-Plex Biomarker assays and analyzed on the MESO QuickPlex SQ120 (Meso Scale Diagnostics).

### Example 10b. T Cell In Vitro Stimulation

Healthy donor primary blood mononuclear cells (PBMCs) were isolated from buffy coats via Ficoll-Paque separation (Global Life Sciences Solutions). Cells were stimulated with 1 μg/mL soluble anti-CD3 (clone OKT3, Biolegend) and 0.2 μg/mL anti-CD28 antibody (clone CD28.2, BD Biosciences) in RPMI media containing 10% Heat Inactivated FBS (Gibco) and 1% Pen/Strep (Gibco).

### Example 11: Generation of the Human IL2 Expression Vector pcDNA3.1/Hygro(+)-huIL2

The human IL2 DNA open reading frame (“ORF”) (Genbank NM_000586.3) was synthesized (Life Technologies GeneArt Service, Carlsbad, CA), and amplified via PCR using Platinum SuperFi II DNA polymerase kit (commercially available as catalog #12361050, ThermoFisher) in substantial accordance with the manufacturer's protocol, and using primers that incorporates an NheI restriction site and an ApaI restriction site. The PCR fragment was visualized on a 1% agarose gel (item #54803, Lonza, Rockland, ME), excised from the gel and purified using a QIAquick PCR Purification kit (commercially available as catalog #28106, Qiagen, Germany) according to the manufacturer's protocol.

The purified PCR fragment and mammalian expression vector pcDNA 3.1/Hygro(+) (commercially available as catalog #V87020, ThermoFisher, Carlsbad CA) were digested with NheI and ApaI (commercially available as catalog #R011 S and #R0114L, New England Biolabs, Ipswich, MA) restriction enzymes. The expression vector was further treated with a Quick Dephosphorylation kit (commercially available as catalog #M0508L, New England Biolabs) in substantial accordance with the manufacturer's protocol. The PCR fragment was ligated into pcDNA 3.1/Hygro(+) using the Rapid DNA Ligation Kit (commercially available as catalog #11635379001, Sigma Aldrich, St. Louis, MO) in substantial accordance with the manufacturer's protocol, transformed into One Shot TOP10 Chemically Competent E. coli (commercially available as catalog #C404006, Life Technologies, Carlsbad, CA), plated onto LB Agar plates containing 100ug/ml carbenicillin (commercially available as catalog #L1010, Teknova, Hollister, CA), and grown overnight at 37 C.

The following day individual bacterial colonies were picked and used to start a 3 ml bacterial culture in LB Broth (#10855-001, Life Technologies) with 100ug/ml ampicillin (commercially available as catalog #A9626, Teknova). The cultures were grown overnight at 37 C. The following day the E. coli were pelleted (6,000 rpm, 10 minutes, tabletop centrifuge #5424, commercially available as catalog Eppendorf, Hauppauge, NY), and the DNA expression vector isolated using QIAprep Spin Miniprep Kit (#27106, Qiagen). The plasmid DNA was sequence verified (MCLab, South San Francisco, CA).

### Example 12. Generation of the Human IL2 REH Expression Vector pcDNA3.1/Hygro(+)-huIL2-REH

An expression vector which introduced three mutations into the human IL2 ORF (L38R, Q42E and Q146H; all numbering based on the full length human IL2 ORF NM_000586.3 numbering, i.e. the hIL2 as expressed including the signal peptide not the 20 amino acid sequence of the mature hIL2 molecule) was assembled in substantial accordance with the teaching of Example 1 with the following exceptions: The initial template DNA used for PCR was synthesized with the L38R (L18R of the mature protein), Q42E (Q22E of the mature protein) and Q146H (Q126H of the mature protein) mutations.

### Example 13. Generation of the Human IL2 REM Expression Vector pcDNA3.1/Hygro(+)-huIL2 REM

An expression vector which introduced three mutations into the human IL2 ORF (L38R, Q42E and Q146M; all numbering based on the full length human IL2 ORF NM_000586.3 numbering) was assembled exactly as described for the human IL2 expression vector in pcDNA3.1/Hygro(+), with the following exceptions: The initial template DNA used for PCR was synthesized with the L38R, Q42E and Q146M mutations.

### Example 14. Introduction of Mutations or Back-Mutations into pcDNA3.1/Hygro(+)-huIL2 and pcDNA3.1/Hygro(+)-huIL2 REH Expression Vectors

All mutations or back-mutations (reverting a mutation in pcDNA3.1 hygro(+)-huIL2-REH back to match the wild type human IL2 ORF) were introduced into the pcDNA3.1 Hygro(+)-huIL2 or pcDNA3.1 Hygro(+)-huIL2-REH expression vectors using a Quik Change II Site Directed Mutagenesis Kit (#200524, Agilent Technologies, Santa Clara, CA) in substantial accordance with the manufacturer's protocol. The transformation of the Quik Change PCR reactions into E. coli, as well as the isolation and sequence analysis of the plasmid DNA, was performed using the same protocol as in the generation of the pcDNA3.1/Hygro-huIL2 expression vector.

### Example 15. Transient Transfections in HEK293 Cells

All expression vectors were transiently transfected into HEK293 cells (#CRL-1573, ATCC, Manassas, VA). -1E6 HEK293 cells were plated into each well of a 6 well tissue culture plate in 2 ml of DMEM (#10569044, Life Technologies) supplemented with 10% Fetal Bovine serum (#SH30071.03, Fisher Scientific, Chicago, IL), and grown overnight at 37 C and 5% CO₂. The next day the cells were transfected using Lipofectamine 3000 Reagent (#L3000150, Life Technologies) following the manufacturer's protocol, using 2.5ug DNA, 5 ul P3000 reagent, and 7.5 ul Lipofectamine 3000 per transfection. The transfected cells were grown at 37 C, 5% CO2 for 48-72 hours and then the conditioned media was harvested.

### Example 16. Analysis of Protein Expression

Protein expression was measured by ELISA using the Human IL2 V-PLEX ELISA kit (#K151QQD-4, Mesoscale Diagnostics, Baltimore, MD) following the manufacturer's protocol (transfected media was diluted 1:4 initially, then 1:2 serially). The plate was read on a Meso Quickplex SQ120 (Mesoscale Diagnostics) using the manufacture's preprogrammed setting for this ELISA kit. The human IL2 standard in the kit was used to compute an approximate expression level in the conditioned media samples.

### Example 17. Determination of IL2 Activity (STAT5) on CD25− and CD25+ Cells

Following a 2-3 day incubation, samples of the supernatants from the 293T containing the soluble IL2 protein cells prepared in accordance with Example 15 above were obtain and added to YT cells (CD25NEG) and YT cells which have been engineered to constituitively express CD25 (YTCD25POS) for a period of approximately 20 minutes. The level of phospho-STAT5 (pSTAT5) induction was measured by flow cytometry. The results of the fold induction of pSTAT5 level is shown in FIG. 12 of the accompanying drawings. Selectivity of the IL2 proteins for CD25 status was calculated as the level of phospho-STAT5 elevation on CD25+YT cells (pSTAT5^{YTCD25}) divided by the level of phospho-STAT5 in CD25 negative YT cells (pSTAT5^{YT}). The results of these experiments are provided in FIG. 13 of the attached drawings. As can be seen from the data presented, the IL2 muteins of the present disclosure provide for selective induction of pSTAT5 on CD25 positive cells and retain significant IL2 activity.

### Example 18. Evaluation of Activity of Orthologs in Human T Cell Clone 3F8

A panel of representative hIL-2 muteins was evaluated for activity in CD4 positive human T cell clone 3F8 cells. The CD4 positive T cell clone 3F8 was generated by activation of PBMC of a healthy donor with the EBV transformed B cell line JY in two successive rounds of Mixed Leukocyte Reactions followed by single cell cloning by limited dilution as described (Yssel and Spits (2002) Current Protocols in Immunology 7.19.1-7.19.12). The CD4 positive T cell clone 3F8 expresses CD25 and CD122 and proliferates and produces IFNγ in response to IL2.

3F8 cells were contacted with supernatants from 293T cells transfected with hIL2 muteins as follows: Cells were grown in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Transferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227) at 0.2 million cells per ml with 50 Gy irradiated JY cells at 0.1 million cells per well and 40 Gy irradiated allogeneic PBMC at 1 million cells per mL. After six days of culture and expansion with human IL-2 at 100 pM, cells were washed and seeded into black, clear bottom 96 well plates (Costar) at 50 thousand cells per well in 75 μl growth medium. Five-fold serial dilutions of transfected 293T cell supernatants were made in growth medium and 75 μl of each dilution was added to plates of 3F8 cells in duplicate at final titrations ranging from 1:2 to 1:78125. Plates were transferred to a humidified incubator (ThermoFisher) and incubated at 37 degrees centigrade, 5 percent carbon dioxide for three days.

Plates were removed from the incubator and 40 μl of culture supernatant was harvested in to a 96 well flat bottom plate (Costar). Supernatants from duplicate wells were pooled. Cells were lysed by adding 100 μl per well of Celltiterglo (Promega) according to manufacturer's instructions. Cell lysates were mixed on an orbital shaker (VWR Scientific) for two minutes at 300 rpm then held at room temperature for 10 minutes. Luminescence for 3F8 cell lysates were read as counts per second on an Envision 2103 Multilabel Plate Reader (Perkin Elmer).

Production of IFNγ in the culture supernatants was measured using the MSD IFNγ V-Plex kit (MSD K151QOD) according to manufacturer's instructions. Briefly, mAb precoated MSD IFNγ assay plates were washed 3 times with 150 L Tris Wash Buffer and IFNγ standards were diluted in Diluent 2. Culture supernatants were diluted 1:1 with Diluent 2 and 50 L of samples and standards were added to the IFNγ assay plates and incubated for 120 min on an orbital shaker (VWR Scientific) at 300 rpm at room temperature. Plates were washed 3 times with Tris Wash Buffer and 25 μL 1×detection antibody in Diluent 3 was added to each well. Plates were incubated for 60 min on an orbital shaker (VWR Scientific) at 300 rpm at room temperature. Plates were washed 3 times with Tris Wash Buffer and 150 L 2× Read Buffer T was added to each well and Luminescence signal was read on a Mesoscale Quickplex SQ120 instrument. Concentration of IFNγ in the supernatants were calculated based on the standard curve with MSD software.

To compare the effect of each hIL-2 mutein upon 3F8 cell proliferation and IFNγ production, CelltiterGlo values and IFNγ concentrations for cells treated with the supernatants were compared to those obtained for control cells treated with growth medium alone, wild-type IL-2 transfection, or supernatant from human REK IL-2 transfection. The data from these experiments is presented in Table 5 and FIG. 16. These data demonstrate correlation between activity of the hIL-2 muteins to induce proliferation and IFNγ production.

SEQUENCE LISTING

| SEQ ID | Description | Sequence |
|---|---|---|
| 1 | Mature Human | ELCDDDPPEIPHATFKAMAYKEGTM |
| | CD25 | LNCECKRGFRRIKSGSLYMLCTGNS |
| | | SHSSWDNQCQCTSSATRNTTKQVTP |
| | | QPEEQKERKTTEMQSPMQPVDQASL |
| | | PGHCREPPPWENEATERIYHFVVGQ |
| | | MVYYQCVQGYRALHRGPAESVCKMT |
| | | HGKTRWTQPQLICTGEMETSQFPGE |
| | | EKPQASPEGRPESETSCLVTTTDFQ |
| | | IQTEMAATMETSIFTTEYQVAVAGC |
| | | VFLLISVLLLSGLTWQRRQRKSRRT |
| | | I |
| | | (SEQ ID NO: 1) |
| 2 | Mature Human | AVNGTSQFTCFYNSRANISCVWSQD |
| | CD122 | GALQDTSCQVHAWPDRRRWNQTCEL |
| | | LPVSQASWACNLILGAPDSQKLTTV |
| | | DIVTLRVLCREGVRWRVMAIQDFKP |
| | | FENLRLMAPISLQVVHVETHRCNIS |
| | | WEISQASHYFERHLEFEARTLSPGH |
| | | TWEEAPLLTLKQKQEWICLETLTPD |
| | | TQYEFQVRVKPLQGEFTTWSPWSQP |
| | | LAFRTKPAALGKDTIPWLGHLLVGL |
| | | SGAFGFIILVYLLINCRNTGPWLKK |
| | | VLKCNTPDPSKFFSQLSSEHGGDVQ |
| | | KWLSSPFPSSSFSPGGLAPEISPLE |
| | | VLERDKVTQLLLQQDKVPEPASLSS |
| | | NHSLTSCFTNQGYFFFHLPDALEIE |
| | | ACQVYFTYDPYSEEDPDEGVAGAPT |
| | | GSSPQPLQPLSGEDDAYCTFPSRDD |
| | | LLLFSPSLLGGPSPPSTAPGGSGAG |
| | | EERMPPSLQERVPRDWDPQPLGPPT |
| | | PGVPDLVDFQPPPELVLREAGEEVP |
| | | DAGPREGVSFPWSRPPGQGEFRALN |
| | | ARLPLNTDAYLSLQELQGQDPTHLV |
| | | (SEQ ID NO: 2) |
| 3 | Mature Human | LNTTILTPNGNEDTTADFFLTTMPT |
| | CD132 | DSLSVSTLPLPEVQCFVFNVEYMNC |
| | | TWNSSSEPQPTNLTLHYWYKNSDND |
| | | KVQKCSHYLFSEEITSGCQLQKKEI |
| | | HLYQTFVVQLQDPREPRRQATQMLK |
| | | LQNLVIPWAPENLTLHKLSESQLEL |
| | | NWNNRFLNHCLEHLVQYRTDWDHSW |
| | | TEQSVDYRHKFSLPSVDGQKRYTFR |
| | | VRSRFNPLCGSAQHWSEWSHPIHWG |
| | | SNTSKENPFLFALEAVVISVGSMGL |
| | | IISLLCVYFWLERTMPRIPTLKNLE |
| | | DLVTEYHGNFSAWSGVSKGLAESLQ |
| | | PDYSERLCLVSEIPPKGGALGEGPG |
| | | ASPCNQHSPYWAPPCYTLKPET |
| | | (SEQ ID NO: 3 |
| 4 | Mature wt | APTSSSTKKTQLQLEHLLLDLQMIL |
| | hIL2 | NGINNYKNPKLTRMLTFKFYMPKKA |
| | | TELKHLQCLEEELKPLEEVLNLAQS |
| | | KNFHLRPRDLISNINVIVLELKGSE |
| | | TTFMCEYADETATIVEFLNRWITFC |
| | | QSIISTLT |
| | | (SEQ ID NO: 4) |
| 5 | Mature wt | APTSSSTSSSTAEAQQQQQQQQQQQ |
| | murine IL2 | QHLEQLLMDLQELLSRMENYRNLKL |
| | | PRMLTFKFYLPKQATELKDLQCLED |
| | | ELGPLRHVLDLTQSKSFQLEDAENF |
| | | ISNIRVTVVKLKGSDNTFECQFDDE |
| | | SATVVDFLRRWIAFCQSIISTSPQ |
| | | (SEQ ID NO: 5) |
| 6 | SQVLKA | APTSSSTKKTQLQLSQLLVLLKAIL |
| | | NGINNYKNPKLTRMLTFKFYMPKKA |
| | | TELKHLQCLEEELKPLEEVLNLAQS |
| | | KNFHLRPRDLISNINVIVLELKGSE |
| | | TTFMCEYADETATIVEFLNRWITFC |
| | | QSIISTLT (SEQ ID NO: 6) |
| 7 | Des-Ala1 | PTSSSTKKTQLQLSQLLVLLKAILN |
| | SQVLKA | GINNYKNPKLTRMLTFKFYMPKKAT |
| | | ELKHLQCLEEELKPLEEVLNLAQSK |
| | | NFHLRPRDLISNINVIVLELKGSET |
| | | TFMCEYADETATIVEFLNRWITFCQ |
| | | SIISTLT (SEQ ID NO: 7) |
| 8 | hIL2 | APTSSSTKKTQLQLEHLRLDLEMIL |
| | L18R/Q22E/ | NGINNYKNPKLTRMLTFKFYMPKKA |
| | Q126K | TELKHLQCLEEELKPLEEVLNLAQS |
| | | KNFHLRPRDLISNINVIVLELKGSE |
| | | TTFMCEYADETATIVEFLNRWITFC |
| | | KSIISTLT |
| | | (SEQ ID NO: 8) |
| 9 | Des Ala1 | PTSSSTKKTQLQLEHLRLDLEMILN |
| | REK | GINNYKNPKLTRMLTFKFYMPKKAT |
| | | ELKHLQCLEEELKPLEEVLNLAQSK |
| | | NFHLRPRDLISNINVIVLELKGSET |
| | | TFMCEYADETATIVEFLNRWITFCK |
| | | SIISTLT |
| | | (SEQ ID NO: 9) |

1-60. (canceled)

61. A method of treating a subject suffering from a neoplastic disease, said method comprising the steps of:
(a) isolating a tissue sample from the subject, the tissue sample comprising a population of TILs;
(b) contacting the isolated tissue sample of step (a) ex vivo with a quantity of an αβhIL2 mutein at a concentration sufficient to induce proliferation and activation of the TILs to generate an expanded cell population comprising activated TILs; and
(c) administering to the subject the expanded cell population comprising activated TILs from step (b).

62. The method of claim 61, wherein the method further comprises a step of administering to the subject a therapeutically effective amount of a second αβhIL2 mutein prior to step (a) and/or after step (c), wherein the first and second αβhIL2 muteins are the same or different.

63. The method of claim 61, wherein the method further comprises the steps of:
(i) administering to the subject a therapeutically effective amount of a αβhIL2 mutein prior to step (a); and
(ii) administering to the subject a therapeutically effective amount of a αβhIL2 mutein following step (c),
wherein each of the αβhIL2 muteins are the same; the αβhIL2 muteins administered in steps (i) and (ii) are the same; the αβhIL2 mutein administered in step (i) and used in step (c) are the same, or each of the αβhIL2 muteins are different αβhIL2 muteins.

64. The method of claim 61, wherein the TTLs comprise antigen activated T cells.

65. The method of claim 61, wherein:
(i) the αβhIL2 mutein is an IL2 mutein having at least 90% sequence identity to wt-hIL2 (SEQ ID NO:4) and comprising an amino acid substitution at position 18, 22, or 126 numbered in accordance with wt-hIL2 (SEQ ID NO:4); or
(ii) the αβhIL2 mutein is an IL2 mutein having at least 90% sequence identity to wt-hIL2 (SEQ ID NO:4) and comprising three amino acid substitutions at position 18, 22, and 126 numbered in accordance with wt-hIL2 (SEQ ID NO:4).

66. The method of claim 65, wherein the αβhIL2 mutein comprises a deletion of 1, 2, 3, 4, 5, 6, 7, 8, or 9 N-terminal amino acids.

67. The method of claim 62, wherein:
(i) each αβhIL2 mutein is an IL2 mutein having at least 90% sequence identity to wt-hIL2 (SEQ ID NO:4) and comprising an amino acid substitution at position 18, 22, or 126 numbered in accordance with wt-hIL2 (SEQ ID NO:4); or
(ii) each αβhIL2 mutein is an IL2 mutein having at least 90% sequence identity to wt-hIL2 (SEQ ID NO:4) and comprising three amino acid substitutions at position 18, 22, and 126 numbered in accordance with wt-hIL2 (SEQ ID NO:4).

68. The method of claim 67, wherein each αβhIL2 mutein comprises a deletion of 1, 2, 3, 4, 5, 6, 7, 8, or 9 N-terminal amino acids.

69. The method of claim 61, wherein the αβhIL2 mutein administered to the subject is modified to extend its duration of action in vivo.

70. The method of claim 61, wherein:
i) the tissue sample is selected from the group consisting of blood and solid tumor tissue; and/or
ii) the subject is treated with a lymphodepleting regimen prior to the administration of the quantity of antigen activated T-cells to the subject; and/or
iii) the step of contacting the isolated population of cells with an αβhIL2 mutein is practiced in combination with one or more additional T-cell activation agent.

71. The method of claim 61, wherein the method is practiced in combination with the administration of a supplementary agent to the subject.

72. The method of claim 61, wherein the method comprises the steps of:
(c) contacting the expanded cell population from step (b) with a recombinant vector comprising a nucleic acid sequence encoding an engineered receptor which is selectively activated in response to the administration of a cognate ligand which binds to the extracellular domain of the engineered receptor and results in intracellular signaling in the T cells expressing the engineered receptor;
(d) administering to the subject the expanded cell population comprising activated TILs from step (c); and
(e) administering to the subject a therapeutically effective amount of a cognate ligand for the engineered receptor.

73. The method of claim 72, wherein:
i) prior to the administration of the cell population to the subject the subject is treated with a lymphodepleting regimen; and/or
ii) prior to the administration of the cell population to the subject the cell population is contacted with a T-cell activation agent; and/or
iii) prior to the isolation of the tissue sample, the subject is pretreated in vivo with a therapeutically effective amount of an αβhIL2 mutein; and/or
iv) the vector is a lentiviral vector or a retroviral vector; and/or
v) the engineered receptor is an hCD122 comprising at least one amino acid substitution at position selected from positions 133 or 134 numbered in accordance with SEQ ID NO:2; and/or
vi) the cognate ligand is a hIL2 variant that selectively binds an hCD122 comprising at least one amino acid substitution at position selected from positions 133 or 134 numbered in accordance with SEQ ID NO:2; and/or
vii) the cognate ligand is modified to extend its duration of action in vivo, optionally wherein the modification to extend the duration of action in vivo is PEGylation, optionally wherein the hIL2 mutein is modified by the N-terminal addition of 40 kDa branched PEG molecule.

74. The method of claim 61, wherein the neoplastic disease disorder or condition is selected from the group consisting of: adenomas, fibromas, hemangiomas, hyperplasia, atypia, metaplasia, dysplasia, carcinomas, leukemias, breast cancers, sarcomas, leukemias, lymphomas, genitourinary cancers, ovarian cancers, urethral cancers, bladder cancers, prostate cancers, gastrointestinal cancers, colon cancers, esophageal cancers, stomach cancers, lung cancers; myelomas; pancreatic cancers; liver cancers; kidney cancers; endocrine cancers; skin cancers; gliomas, neuroblastomas, astrocytomas, myelodysplastic disorders; cervical carcinoma-in-situ; intestinal polyposes; oral leukoplakias; histiocytoses, hyperprofroliferative scars including keloid scars, respiratory system carcinomas, gastrointestinal system carcinomas, genitourinary system carcinomas, testicular carcinomas, breast carcinomas, prostatic carcinomas, endocrine system carcinomas, melanomas, adenocarcinomas, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage, promyeloid leukemia (APML), acute myelogenous leukemia (AML) and chronic myelogenous leukemia (CMIL), precursor lymphoid neoplasms, mature B-cell neoplasms, mature T-cell neoplasms, Hodgkin's Lymphoma, and immunodeficiency-associated lymphoproliferative disorders, lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). erythroblastic leukemia and acute megakaryoblastic leukemia, malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), and Hodgkin's disease.

75. An ex vivo method of preparing a population of antigen activated T cells, the method comprising contacting a tissue sample isolated from a subject, wherein the tissue sample comprises a population of TILs, with a quantity of an αβhIL2 mutein at a concentration sufficient to induce proliferation and activation of the TILs to generate an expanded cell population comprising activated TILs.

76. The method of claim 75, further comprising a step of contacting the cell product with a recombinant vector comprising a nucleic acid sequence encoding an engineered receptor which is selectively activated in response to the administration of a cognate ligand which binds to the extracellular domain of the engineered receptor and results in intracellular signaling in the T cells expressing the engineered receptor, optionally wherein:
i) vector is a lentiviral vector or a retroviral vector; and/or
ii) the engineered receptor is an hCD122 comprising at least one amino acid substitution at position selected from positions 133 or 134 numbered in accordance with SEQ ID NO:2; optionally wherein the engineered receptor is an hCD122 comprising amino acid substitutions at positions 133 and 134, optionally wherein the hCD122 comprising amino acid substitutions H133D and Y134F.

77. A cell product produced by the method of claim 75.

78. A method of treating a subject suffering from a neoplastic disease, the method comprising administering to the subject the cell product of claim 77.

79. Use of an αβhIL2 mutein ex vivo for the activation and expansion of antigen activated T-cells.
